(19) United States
(12) Patent Application Publication
(10) Pub. No.: US 2025/0059258 A1
(43) Pub. Date: Feb. 20, 2025

(54) CH1 DOMAIN AFFINITY LIGANDS AND AGENTS

(71) Applicant: AVITIDE LLC, Lebanon, NH (US)

(72) Inventors: Thomas Scanlon, Lebanon, NH (US); Jonathan Belk, Grantham, NH (US); Kelley Kearns, Grantham, NH (US); Sarah Valentini, Newport, NH (US)

(73) Assignee: AVITIDE LLC, Lebanon, NH (US)

(21) Appl. No.: 18/721,454

(22) PCT Filed: Dec. 23, 2022
(86) PCT No.: PCT/US2022/053926

§ 371 Date: Jun. 18, 2024

Related U.S. Application Data
(60) Provisional application No. 63/293,718, filed on Dec. 24, 2021.
(60) Provisional application No. 63/308,707, filed on Feb. 10, 2022.
(60) Provisional application No. 63/407,563, filed on Sep. 16, 2022.

(51) Int. Cl. C07K 16/00; C07K 1/22; C07K 14/00; C07K 17/08

(52) U.S. Cl. CPC C07K 16/00; C07K 1/22; C07K 14/001; C07K 17/08

(57) ABSTRACT

The present disclosure relates to the field of chromatography, and more specifically to novel affinity ligands and affinity agents which are suitable for use in isolation of antibody and antibody fragments that contain the heavy chain first constant region (CHI). The disclosure encompasses affinity ligands as such, chromatography separation matrices (affinity agents) comprising an affinity ligand according to the disclosure, and a process of isolating antibodies, especially IgGs, and CHI-containing antibody fragments thereof using an affinity ligand according to the disclosure. Methods of making the affinity ligands and separation matrices are also provided.

### CROSS REFERENCE TO RELATED APPLICATIONS

This PCT application claims benefit of U.S. Provisional Application No. 63/293,718 filed on Dec. 24, 2021, U.S. Provisional Application No. 63/308,707 filed on Feb. 10, 2022, and U.S. Provisional Application No. 63/407,563 filed on Sep. 16, 2022, the entire contents of each of which are incorporated by reference herein.

### FIELD OF THE DISCLOSURE

The present disclosure relates to the field of chromatography, and more specifically to novel affinity ligands and affinity agents which are suitable for use in isolation of antibody and antibody fragments that contain the heavy chain first constant region (CH1). The disclosure encompasses affinity ligands as such, chromatography separation matrices (affinity agents) comprising an affinity ligand according to the disclosure, and a process of isolating antibodies, especially IgGs, and CH1-containing antibody fragments thereof using an affinity ligand according to the disclosure. Methods of making the affinity ligands and separation matrices are also provided.

### BACKGROUND OF THE DISCLOSURE

The purity of biologically produced therapeutics is tightly scrutinized and regulated by authorities to ensure safety and efficacy. Thus, there is a need to efficiently purify biologically produced therapeutics to a high degree of purity.

To support the clinical efforts for therapeutic proteins, compositions and methods to efficiently purify proteins from recombinant sources are needed. Affinity purification is a means to isolate and/or achieve desired purity of a protein in few steps, or a single step. However, the development of affinity agents (e.g., comprising an affinity ligand) can be a resource intensive and time-consuming task and hence affinity agents exist for very few proteins. In the absence of an affinity agent, purification typically involves inefficient processes, such as a multi-column process.

Exemplary therapeutic proteins include, but are not limited to, bioactive polypeptides/proteins, fusion proteins, enzymes, hormones, antibodies, and antibody fragments. Certain proteins, such as fusion proteins, present additional challenges for purification due to product homogeneity, or the existence of product related impurities. Some impurities may arise from incorrectly assembled fusion proteins and proteolytic cleavage which can be especially difficult to remove because they are closely related to the desired product.

Affinity agents that bind proteins and are useful for isolation and/or affinity purification are described herein. In some embodiments, an affinity agent comprises a solid support and a ligand.

Human monoclonal antibody therapy is a rapidly growing segment of the biologic pharmaceutical industry with very promising clinical applications in cancer therapy. Traditional methods for recombinant antibody purification utilize affinity chromatography resins based on Protein A ligands. Protein A based affinity resins provide highly efficient, high yield processes for purification of monoclonal antibodies, their utility in industrial antibody production processes highlights a need for suitable affinity purification devices.

A major drawback of monoclonal antibody therapy based on immunoglobulin G (IgG) is poor tumor penetration by the very large IgG molecule (˜150 kDa). Increasingly, antibody fragments are being investigated that lack the large constant domain (Fc domain) of IgGs. Antibody fragments thus constructed maintain their native antigen-specificity but lack the ability to bind the Fc receptor. However, such antibody fragments also lack the Protein A binding site that enables purification with Protein A-based affinity resins, and thus new methods are needed for purification of antibody fragments that cannot bind to Protein A resins.

Exemplary antibody fragment formats currently being investigated in clinical trials include, for example, monospecific Fab, monospecific Fab₂, bispecific Fab, bispecific Fab₂, mono and bi-specific diabody, triabody, antibody fragment—drug conjugates, etc. (reviewed in Nelson, 2010, mAbs, and others).

Many of the aforementioned antibody fragments contain a conserved region on the heavy chain, the CH1 domain. Affinity agents that bind antibodies or antibody fragments in the CH1 domain would be useful for isolation and/or affinity purification of such fragments. Affinity agents that bind antibodies or antibody fragments and are useful for isolation and/or purification are described herein. In some embodiments an affinity agent comprises a solid support and a ligand.

The tetratricopeptide repeat (TPR) proteins offer a potential scaffold to engineer binding specificities and create new binding ligands suitable for such affinity purification. TPRs contain repeating units of a 34-amino-acid sequence that adopts a helix-turn-helix structure. Combining three such domains with a seventh helix provides forms an elongated superhelical structure where helices 1, 3, 5 and 7 form an inner surface of the superhelix and the binding interface for its target (see FIG. 1). In accordance with this disclosure, a series of affinity ligands based on non-naturally occurring TPRs have been developed for binding antibodies or antibody fragments in the CH1 domain. Moreover, the resulting TPR domain-based ligands have high stability including the ability to withstand CIP with NaOH. Collectively, these properties make possible the use of affinity ligands that can withstand the rigors of modern bioprocessing.

### SUMMARY OF THE DISCLOSURE

The present disclosure provides affinity ligands which bind antibodies and antibody fragments which have or comprise a CH1 domain. Such affinity ligands comprise an amino acid sequence represented by the formula, from N-terminus to C-terminus,

[A]-AERWYDLGAAYAARGDX₁₇ₐDRAX₂₁ₐEX₂₃ₐYX₂₅ₐRX₂₇ₐLEX₃₀ₐ
DPND-AWAWWELGX_{9b}AYAARGDYDRAIEYYQRALELDX_{32b}NN-
AVAWARLGIAYAX_{13c}RGDYDRAIEYYQRALELDPNN-
EVARX_{5d}ALEYARRX_{13d}-[B]

wherein X₁₇ₐ is F or Y; X₂₁ₐ is I or T; X₂₃ₐ is Y or F; X₂₅ₐ is R or Q; X₂₇ₐ is T or A; and X₃₀ₐ is L or H; wherein X_{9b} is V or I; and X_{32b} is L, P or T; wherein X_{13c} is T or A; wherein X_{5d} is D or G and X_{13d} is A or V;
wherein [A] is present or absent, and when present comprises M, MDE, MGGGGSAAAGDE, MGDE, MGHHHHHHDE, MGLAEAAAREAAARAADE, or MAWAEFRQRLAAIRTRLEALGGSEAELAAFEREIAAFESELQAYAGAGNPEVEALRREA AAIRDELQAYRHNDE; and wherein [B] is present or absent, and when present comprises a cysteine, RRC, RRCHHHHHH, or RRGQAGQGGGSGLNDIFEAQKIEWHECHHHHHH.

In some embodiments of the affinity ligands, X₁₇ₐ is F; X₂₁ₐ is I; X₂₃ₐ is Y; X₂₅ₐ is R; X₂₇ₐ is T; X₃₀ₐ is L; X_{9b} is V; X_{32b} is P; X_{13c} is T; X_{5d} is D; and X_{13d} is V.

In some embodiments of the affinity ligands, X₁₇ₐ is Y; X₂₁ₐ is T; X₂₃ₐ is F; X₂₅ₐ is Q; X₂₇ₐ is A; X₃₀ₐ is L; X_{9b} is I; X_{32b} is P; X_{13c} is A; X_{5d} is D; and X_{13d} is A.

In some embodiments of the affinity ligands, X₁₇ₐ is Y; X₂₁ₐ is I; X₂₃ₐ is Y; X₂₅ₐ is Q; X₂₇ₐ is A; X₃₀ₐ is H; X_{9b} is V; X_{32b} is T; X_{13c} is A; X_{5d} is G; and X_{13d} is A.

In some embodiments of the affinity ligands, X₁₇ₐ is Y; X₂₁ₐ is I; X₂₃ₐ is Y; X₂₅ₐ is Q; X₂₇ₐ is A; X₃₀ₐ is L; X_{9b} is V; X_{32b} is L; X_{13c} is A; X_{5d} is D; and X_{13d} is A.

In certain embodiments [A] is MGHHHHHHDE and [B] is RRC. In certain embodiments, [A] is MGLAEAAAREAAARAADE and [B] is RRCHHHHHH.

In some embodiments the affinity ligands comprise any of those in Table 9.

In preferred embodiments, the affinity ligands comprise any one of SEQ ID NOS: 89, 128, 129, 130, 131 or 132 (“the ligand 129 group”).

In preferred embodiments, the affinity ligands comprise any one of SEQ ID NOS: 90, 133, 134, 135, 136 or 137 (“the ligand 134 group”)

In preferred embodiments, the affinity ligands comprise any one of SEQ ID NOS: 139, 140, 141 or 143 (“the ligand 141 group”).

In preferred embodiments, the affinity ligands comprise any one of SEQ ID NOS: 86, 91, 138, 142, 144, 145, 146, 147 or 148 (“the ligand 144 group”).

In more preferred embodiments, the affinity ligands comprise any one of SEQ ID NOS: 129, 134, 141 or 144.

In some embodiments, an affinity ligand of the disclosure comprises a C-terminal cysteine or lysine.

Further aspects of the disclosure relate to multimers comprising a plurality of affinity ligands according to the disclosure. Such multimers include dimers, trimers, tetramers, pentamers, hexamers, heptamers, octamers and nonamers.

In other aspects described herein, an affinity ligand or multimer disclosed herein further comprises at least one heterologous agent operably linked to said affinity ligand or multimer to form a conjugate or a fusion protein.

Yet other aspects of the disclosure relate to separation matrices. Such separation matrices comprise at least one affinity ligand of the disclosure or at least one multimer of the disclosure. In embodiments, the ligands or multimers are coupled to a solid support, optionally via thiol linkages.

Further still, the disclosure provides methods of isolating an antibody comprising a CH1 domain, or a fragment thereof comprising a CH1 domain, which comprises contacting said antibody or said fragment with a separation matrix of the disclosure.

In some embodiments the method is directed to isolating or purifying an antibody comprising a CH1 domain, or a fragment thereof comprising a CH1 domain, which method comprises (a) contacting a separation matrix of the disclosure with a composition comprising the said antibody or said fragment, (b) washing said separation matrix with a washing buffer, (c) eluting said antibody or said fragment from the separation matrix with an elution buffer, and (d) recovering said antibody or said fragment.

In some embodiments, the method which further comprises (e) treating the separation matrix with an alkaline cleaning solution for a time sufficient to clean said matrix of residual material and to regenerate at least 80% of the antibody- or the antibody fragment-binding capacity of said separation matrix. In some embodiments, the alkaline cleaning solution comprises from about 0.1 M NaOH to about 0.5 M NaOH. For example, the separation matrix can retain at least 80% of its antibody- or the antibody fragment-binding capacity when steps (a)-(e) are repeated at least 10 times. In some embodiments, binding capacities of at least 85, 90 and 95% are retained. As such the same column can be repeatedly use for purification, up to at least 10 times or more provided the binding capacity of the resin is retained. Such separation matrices are alkaline stable and provide reduce the purification costs.

Further aspects of the disclosure related to nucleic acids or vectors encoding an affinity ligand of the disclosure or a multimer of the disclosure as well as expression vector comprising those nucleic acids or vectors having the coding region of the affinity ligand or multimer operably linked to one or more expression control elements. Additional embodiments of the disclosure are directed to host cells, particularly, E. coli or P. pastoris for recombinant production of an affinity ligand or multimer of the disclosure.

In some embodiments, the disclosure relates to methods of making a separation matrix comprising conjugating a ligand according to the disclosure or a multimer according to the disclosure to a solid surface.

### BRIEF DESCRIPTION OF THE DRAWINGS

FIG. 1 shows a general ribbon structure for a TPR domain having seven alpha helices as exemplified by the affinity ligands of the disclosure. The 7 alpha helices are labelled consecutively, α1-α7, beginning at the N-terminus. The N- and C-termini are also indicated by “N” and “C”, respectively.

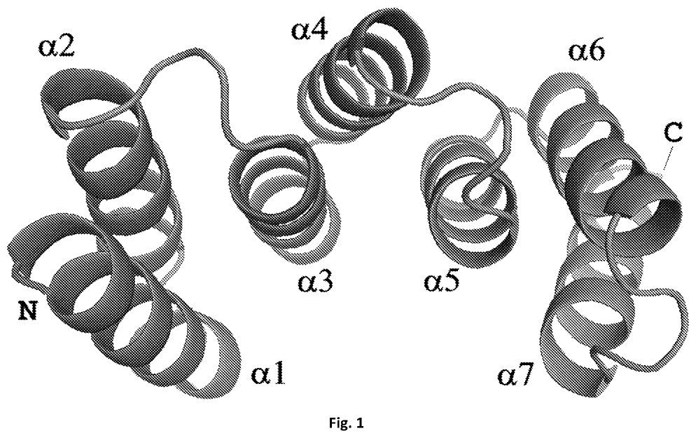

FIG. 2 shows sensorgrams for exemplary affinity agents. Sensorgrams are for the biotinylated ligands corresponding to SEQ ID No. 89, SEQ ID No. 90, and SEQ ID No. 92 titrated with the indicated concentrations of Herceptin Fab in solution (dark blue, maroon and light blue lines). The light red lines are best fit curves.

FIG. 3 shows sensorgrams for exemplary affinity agents. Sensorgrams are for the biotinylated ligands corresponding to SEQ ID No. 89, SEQ ID No. 90, and SEQ ID No. 92 titrated with the indicated concentrations of Tremfya Fab in solution (dark blue, maroon and light blue lines). The light red lines are best fit curves.

FIG. 4 shows the adsorption isotherms of resin prepared from SEQ ID No. 129 before (▪) and after (●) incubation in 0.1 M NaOH+1 M NaCl for 24 hours.

FIG. 5 shows the adsorption isotherms of resin prepared from SEQ ID No. 134 before (▪) and after (●) incubation in 0.1 M NaOH+1 M NaCl for 24 hours.

FIG. 6 shows the adsorption isotherms of resin prepared from SEQ ID No. 144 before (▪) and after (●) incubation in 0.1 M NaOH+1 M NaCl for 24 hours.

FIG. 7 shows an SDS-PAGE gel run under non-reducing conditions for various fractions obtained from a column purification run of a monoclonal antibody produced in a CHO cell line using an affinity resin with the ligand of SEQ ID No. 89. The samples are in each lane are (1) SeeBlue Plus2 molecular weight markers, (2) crude feedstream (also known as CCCF), (3) 10×-diluted crude feedstream, (4) flow through fraction pool, (5) Wash fraction pool, (6) elution fraction pool (6.4 ug), and (7) strip fraction pool.

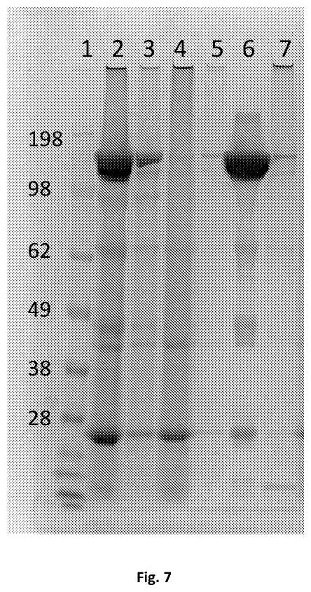

FIG. 8 shows the residual HCP (top panel) and residual HCDNA (bottom panel) measured across several cycles of the purification of a monoclonal antibody produced in a CHO cell line the affinity resins with the ligands of SEQ ID No. 129 (blue lines), 134 (red lines) and 144 (yellow lines).

FIG. 9 shows the yield measured across several cycles of the purification of an IgG antibody as described in Example 8.

FIG. 10 shows the ForteBio dose response of the binding of Trastuzumab and Trastuzufab protein to biotinylated ligand corresponding to SEQ ID No: 157 immobilized on sensors.

FIG. 11 shows the breakthrough curves obtained during a DBC determination of an affinity resin prepared with the ligand corresponding to SEQ ID No: 181 and packed into a 3×50 mm column. The breakthrough curves are shown for Trastuzumab (Upper graph) and Trastuzufab, Nucala® and Benlysta® (Lower graph) obtained at 4 minute residence time using a challenge concentration of 2 g/L.

FIG. 12 shows the stability of the DBC towards exposure with NaOH. An affinity resin prepared with the ligand corresponding to SEQ ID No: 181 was exposed to 1 M NaOH containing 1 M NaCl and the DBC was determined at the time points indicated.

FIG. 13 shows the relative yield obtained for each cycle during the purification of Trastuzumab from crude HCCF with repetitive cycling of the column. The column was exposed to 1 M NaOH containing 1 M NaCl for 3.5 hours at the end of each cycle.

FIG. 14 shows the residual HCP obtained for each cycle during the purification of Trastuzumab from crude HCCF with repetitive cycling of the column. The column was exposed to 1 M NaOH containing 1 M NaCl for 3.5 hours at the end of each cycle.

FIG. 15 shows the residual HCP obtained for each cycle during the purification of Trastuzumab from crude HCCF with repetitive cycling of the column. The column was exposed to 1 M NaOH containing 1 M NaCl for 3.5 hours at the end of each cycle.

### DETAILED DESCRIPTION OF THE DISCLOSURE

### Definitions

In order for the present disclosure to be more readily understood, certain terms are defined below. Unless defined otherwise herein, technical and scientific terms used herein have the same meaning as commonly understood by one of ordinary skill in the art to which this disclosure is related.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the disclosure, which can be had by reference to the specification as a whole. Accordingly, the terms defined immediately below are more fully defined by reference to the specification in its entirety.

It is to be noted that the term “a” or “an” entity refers to one or more of that entity; for example, “an affinity ligand” is understood to represent one or more affinity ligands. As such, the terms “a” (or “an”), “one or more,” and “at least one” can be used interchangeably herein.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the term “biologically active” refers to a characteristic of any agent that has activity in a biological system, and particularly in an organism. For instance, an agent that, when administered to an organism, has a biological or physiological effect on that organism, is considered to be biologically active.

Variant and Mutant: The term “variant” is usually defined in the scientific literature and used herein in reference to an organism that differs genetically in some way from an accepted standard, “Variant” can also be used to describe phenotypic differences that are not genetic (King and Stansfield, 2002, A dictionary of genetics, 6th ed., New York, New York, Oxford University Press.

The term “mutation” is defined by most dictionaries and used herein in reference to the process that introduces a heritable change into the structure of a gene (King & Stansfield, 2002) thereby producing a “mutant.” The term “variant” is increasingly being used in place of the term “mutation” in the scientific and non-scientific literature. The terms are used interchangeably herein.

Conservative and non-conservative substitution: A “conservative” amino acid substitution is one in which one amino acid residue is replaced with another amino acid residue having a similar side chain. Families of amino acid residues having similar side chains have been defined in the art, including basic side chains (e.g., lysine (K), arginine (R), histidine (H)); acidic side chains (e.g., aspartic acid (D), glutamic acid (E)); uncharged polar side chains (e.g., glycine (G); asparagine (N), glutamine (Q), serine (S), threonine (T), tyrosine (Y), cysteine (C)); nonpolar side chains (e.g., alanine (A), valine (V), leucine (L), isoleucine (I), proline (P), phenylalanine (F), menine (M), tryptophan (W), beta-branched side chains (e.g., threonine (T), valine (V), isoleucine (I)); and aromatic side chains (e.g., tyrosine (Y), phenylalanine (F), tryptophan (W), histidine (H)). For example, substitution of a phenylalanine for a tyrosine is a conservative substitution. In some embodiments, conservative amino acid substitutions in the sequence of a ligand confer or improve specific binding of the ligand a target of interest. In some embodiments, conservative amino acid substitutions in the sequences of a ligand do not reduce or abrogate the binding of the ligand to a target of interest. In some embodiments, conservative amino acid substitutions do not significantly affect specific binding of a ligand to a target of interest. Methods of identifying nucleotide and amino acid conservative substitutions and non-conservative substitutions which confer, alter or maintain selective binding affinity are known in the art (see, e.g., Brummell, Biochem. 32:1180-1187 (1993); Kobayashi, Protein Eng. 12(10):879-884 (1999); and Burks, PNAS 94:412-417 (1997)). In some embodiments, non-conservative amino acid substitutions in the sequence of a ligand confer or improve specific binding of the ligand a target of interest. In some embodiments, non-conservative amino acid substitutions in the sequences of a ligand do not reduce or abrogate the binding of the ligand to a target of interest. In some embodiments, non-conservative amino acid substitutions do not significantly affect specific binding of a ligand to a target of interest.

Affinity chromatography: As used herein the term “affinity chromatography” refers to the specific mode of chromatography in which an affinity ligand interacts with a target via biological affinity in a “lock-key” fashion. Examples of useful interactions in affinity chromatography are e.g., enzyme-substrate interaction, biotin-avidin interaction, antibody-antigen interaction, etc.

Affinity ligand and Ligand: The terms “affinity ligand” and “ligand” are used interchangeably herein. These terms are used herein to refer to molecules that are capable of reversibly binding with high affinity to a moiety specific for it, e.g., a polypeptide or protein.

Protein-based ligand: The term “protein-based ligands” as used herein means ligands which comprise a peptide or protein or a part of a peptide or protein that binds reversibly to a target polypeptide or protein. It is understood that the “ligands” of the disclosure are protein-based ligands.

Affinity agent: As used herein, the term “affinity agent” is in reference to a solid support or matrix to which a biospecific affinity ligand is covalently attached. Typically, the solid support or matrix is insoluble in the system in which the target molecule is purified. The terms “affinity agent” and “affinity separation matrix(ces)” and “separation matrix(ces)” are used interchangeably herein.

Linker: As used herein a “linker” refers to a peptide or other chemical linkage that functions to link otherwise independent functional domains. In some embodiments, a linker is located between a ligand and another polypeptide component containing an otherwise independent functional or structural domain. In some embodiments, a linker is a peptide or other chemical linkage located between a ligand and a surface.

Naturally occurring: The term “naturally occurring” when used in connection with biological materials such as a nucleic acid molecules, polypeptides, and host cells, refers to those which are found in nature and not modified by a human being. Conversely, “non-natural” or “synthetic” when used in connection with biological materials refers to those which are not found in nature and/or have been modified by a human being.

“Non-natural amino acids,” “amino acid analogs” and “non-standard amino acid residues” are used interchangeably herein. Non-natural amino acids that can be substituted in a ligand as provided herein are known in the art. In some embodiments, a non-natural amino acid is 4-hydroxyproline which can be substituted for proline; 5-hydroxylysine which can be substituted for lysine; 3-methylhistidine which can be substituted for histidine; homoserine which can be substituted for serine; and ornithine which can be substituted for lysine. Additional examples of non-natural amino acids that can be substituted in a polypeptide ligand include, but are not limited to molecules such as: D-isomers of the common amino acids, 2,4-diaminobutyric acid, alpha-amino isobutyric acid, A-aminobutyric acid, Abu, 2-amino butyric acid, gamma-Abu, epsilon-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, beta-alanine, lanthionine, dehydroalanine, γ-aminobutyric acid, selenocysteine and pyrrolysine fluoro-amino acids, designer amino acids such as beta-methyl amino acids, C alpha-methyl amino acids, and N alpha-methyl amino acids.

“Polynucleotide” and “nucleic acid molecule”: As used interchangeably herein, polynucleotide and nucleic acid molecule refer to a polymeric form of nucleotides of any length, either ribonucleotides or deoxyribonucleotides. These terms include, but are not limited to, DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA (transfer RNA).

Operably linked: The term “operably linked,” as used herein, indicates that two or more components are arranged such that the components function normally and allow the possibility that at least one of the components can mediate a function that is exerted upon at least one of the other components. Two molecules are “operably linked” whether they are attached directly or indirectly.

Peptide tag: The term “peptide tag” as used herein refers to a peptide sequence that is part of or attached (for instance through genetic engineering) to another protein, to provide a function to the resultant fusion. Such functions include but are not limited to, altering solubility of the protein, moderating expression of the protein, and facilitating attachment or interaction of the protein to another entity. Peptide tags are usually, but not always, relatively short in comparison to a protein to which they are fused. In some embodiments, a peptide tag is four or more amino acids in length, such as, 5, 6, 7, 8, 9, 10, 15, 20, or 25 or more amino acids. In some embodiments, a peptide tag as used herein, includes a second protein that can act as a “tag” (e.g., GFP) or facilitator of a particular property. In some embodiments, a ligand is a protein that contains a peptide tag. Numerous peptide tags that have uses as provided herein are known in the art. Examples of peptide tags that may be a component of a ligand fusion protein or a target bound by a ligand (e.g., a ligand fusion protein) include but are not limited to HA (hemagglutinin), c-myc, the Herpes Simplex virus glycoprotein D (gD), T7, GST, GFP, MBP, Strep-tags, His-tags, Myc-tags, TAP-tags and FLAG tag (Eastman Kodak, Rochester, N.Y.) Likewise, antibodies to the tag epitope allow detection and localization of the fusion protein in, for example, affinity purification, Western blots, ELISA assays, and immunostaining of cells.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Specifically binds: As used herein in reference to ligands, the term “specifically binds” or “has selective affinity for” means a ligand reacts or associates more frequently, more rapidly, with greater duration, with greater affinity, or combinations of the above to a particular epitope, protein, or target molecule than with alternative substances, including unrelated proteins. Because of the sequence identity between homologous proteins in different species, specific binding can include a binding agent that recognizes a protein or target in more than one species, e.g., is bi- or tri-specific. Likewise, because of homology within certain regions of polypeptide sequences of different proteins, specific binding can include a binding agent that recognizes more than one protein or target. It is understood that, in certain embodiments, a binding agent that specifically binds a first target may or may not specifically bind a second target. As such, “specific binding” does not necessarily require (although it can include) exclusive binding, i.e., binding to a single target. Thus, a ligand or affinity agent may, in certain embodiments, specifically bind more than one target. In certain embodiments, multiple targets may be bound by the same binding site on an affinity agent.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### CH1 Domain Affinity Ligands

The affinity ligands of the various aspects and embodiments of the disclosure are high affinity protein ligands that reversibly bind antibody and antibody fragments that contain the heavy chain first constant region (CH1) (referred to herein as a “CH1 domain”). Such binding allows these protein ligands to be used, inter alia, to isolate and/or purify antibodies and any antibody fragment lacking an Fc domain but retaining the CH1 region of the heavy chain.

The affinity ligands of the disclosure comprise an amino acid sequence represented by the formula from N-terminus to C-terminus

[A]-AERWYDLGAAYAARGDX₁₇ₐDRAX₂₁ₐEX₂₃ₐYX₂₅ₐRX₂₇ₐLEX₃₀ₐ
DPND-AWAWWELGX_{9b}AYAARGDYDRAIEYYQRALELDX_{32b}NN-
AVAWARLGIAYAX_{13c}RGDYDRAIEYYQRALELDPNN-
EVARX_{5d}ALEYARRV-[B]

wherein X₁₇ₐ is F or Y; X₂₁ₐ is I or T; X₂₃ₐ is Y or F; X₂₅ₐ is R or Q; X₂₇ₐ is T or A; X₃₀ₐ is L or H;
wherein X_{9b} is V or I; and X_{32b} is L, P or T;
wherein X_{13c} is T or A;
wherein X₅ₐ is D or G and X_{13d} is A or V; and
wherein [A] is present or absent, and when present comprises M, MDE, MGGGGSAAAGDE, MGDE, MGHHHHHHDE, MGLAEAAAREAAARAADE, or MAWAEFRQRLAAIRTRLEALGGSEAELAAFEREIAAFESELQAYAGAGNPEVEALRREA AAIRDELQAYRHNDE; and [B] is present or absent, and when present comprises a cysteine, RRC, RRCHHHHHH, or RRGQAGQGGGSGLNDIFEAQKIEWHECHHHHHH. These affinity ligands bind antibody and antibody fragments that comprise a CH1 domain.

The affinity ligands of the disclosure are derived from TPR-domain containing proteins having three repeats of a 34 amino acid TPR domain followed by a seventh helix. Each repeat encodes a helix-turn-helix motif typical of those that occur in TPR-domain containing proteins. For clarity, the three 34 amino acid domains are colinear in the ligand but are presented on a single line such that the first amino acid on that line is amino acid 1 of that 34 amino acid domain. Thus, the first TPR domain (as shown above) contains helix 1 and helix 2, the second TPR domain contains helix 3 and helix 4 and the third TPR domain contains helix 5 and helix 6. In this disclosure, “a”, “b” and “c” are also used to denote the three TPR domains in these affinity ligands.

In some embodiments, an affinity ligand of the disclosure has the general formula above wherein X₁₇ₐ is F; X₂₁ₐ is I; X₂₃ₐ is Y; X₂₅ₐ is R; X₂₇ₐ is T; X₃₀ₐ is L; X_{9b} is V; X_{32b} is P; X_{13c} is T; X₅ₐ is D; and X_{13d} is V. In some embodiment, these affinity ligands include, but are not limited to, those comprising SEQ ID NOS. 89, 128, 129, 130, 131 or 132. Such embodiments are also referred to as the Ligand 129 group.

In some embodiments, an affinity ligand of the disclosure has the general formula above wherein X₁₇ₐ is Y; X₂₁ₐ is T; X₂₃ₐ is F; X₂₅ₐ is Q; X₂₇ₐ is A; X₃₀ₐ is L; X_{9b} is I; X_{32b} is P; X_{13c} is A; X₅ₐ is D; and X_{13d} is A. In some embodiment, these affinity ligands include, but are not limited to, those comprising SEQ ID NOS. 90, 133, 134, 135, 136 or 137. Such embodiments are also referred to as the Ligand 134 group.

In some embodiments, an affinity ligand of the disclosure has the general formula above wherein X₁₇ₐ is Y; X₂₁ₐ is I; X₂₃ₐ is Y; X₂₅ₐ is Q; X₂₇ₐ is A; X₃₀ₐ is H; X_{9b} is V; X_{32b} is T; X_{13c} is A; X₅ₐ is G; and X_{13d} is A. In some embodiment, these affinity ligands include, but are not limited to, those comprising SEQ ID NOS. 139, 140, 141 or 143. Such embodiments are also referred to as the Ligand 141 group.

In some embodiments, an affinity ligand of the disclosure has the general formula above wherein X₁₇ₐ is Y; X₂₁ₐ is I; X₂₃ₐ is Y; X₂₅ₐ is Q; X₂₇ₐ is A; X₃₀ₐ is L; X_{9b} is V; X_{32b} is L; X_{13c} is A; X₅ₐ is D; and X_{13d} is A. In some embodiment, these affinity ligands include, but are not limited to, those comprising SEQ ID NOS. 86, 91, 138, 142 or 144-148. Such embodiments are also referred to as the Ligand 144 group.

In some embodiments of the affinity ligand, [A] is MGHHHHHHDE and [B] is RRC. In some embodiments of the affinity ligand, [A] is MGLAEAAAREAAARAADE and [B] is RRCHHHHHH.

In some embodiments, provided herein are affinity agents comprising a TPR ligand comprising 7 helices in which the 1^{st} helix comprises the sequence SEQ ID NO: 1
X¹RWX²X³, where X¹ is A, D, E, G, N, Q, S or V; X² is A, E, W or Y; X³ is D, G, I, N, T, V, W or Y;
the 3^{rd} helix comprises the sequence SEQ ID NO: 2
X⁴AWX⁵X⁶LGX⁷ where X⁴ is W; X⁵ is E or W; X⁶ is D, E or H; X⁷ is T, V, W;
the 5^{th} helix comprises the sequence SEQ ID NO: 3
X⁸AWX⁹X¹⁰LGX¹¹, where X⁸ is A, H, I, T or V; X⁹ is A, F, N, S or R; X¹⁰ is R; X¹¹ is F, I, H or W; and
the 7^{th} helix comprises the sequence SEQ ID NO: 4
X¹²ARX¹³X¹⁴LEX¹⁵, where X¹² is A, L, T or V; X¹³ is A, D, E, H, Q, R, S or Y; X¹⁴ is A, E, I, L, N or Q; X¹⁵ is A, D, E, H, N, R, S, V or Y.

In some embodiments, provided herein are affinity agents comprising a TPR ligand comprising 7 helices in which the 1^{st} helix comprises the sequence SEQ ID NO: 1
X¹RWX²X³, where X¹ is D or E, Q or V; X² is E or Y; X³ is D, I or N; the 3^{rd} helix comprises the sequence SEQ ID NO: 2
X⁴AWX⁵X⁶LGX⁷, where X⁴ is W; X⁵ is W; X⁶ is E; X⁷ is I or V;
the 5^{th} helix comprises the sequence SEQ ID NO: 3
X⁸AWX⁹X¹⁰LGX¹¹, where X⁸ is H, T or V; X⁹ is A, F or S; X¹⁰ is R; X¹¹ is H, I, or W; and
the 7^{th} helix comprises the sequence SEQ ID NO: 4
X¹²ARX¹³X¹⁴LEX¹⁵, where X¹² is A, L, T or V; X¹³ is D, H, R, or Y: X¹⁴ is A, I, V or L; X¹⁵ is D, H, V or Y.

In some embodiments, provided herein are affinity agents comprising a TPR ligand comprising 7 helices in which the 1 helix comprises the sequence SEQ ID NO: 1
X¹RWX²X³, where X¹ is N; X² is Y; X³ is V or Y;
the 3^{rd} helix comprises the sequence SEQ ID NO: 2
X⁴AWX⁵X⁶LGX⁷, where X⁴ is F, or Y; X⁵ is D; X⁶ is E; X⁷ is N or S;
the 5^{th} helix comprises the sequence SEQ ID NO: 3
X⁸AWX⁹X¹⁰LGX¹¹, where X⁸ is V; X⁹ is N, or S; X¹⁰ is R; X¹¹ is H, or Q and the 7^{th} helix comprises the sequence SEQ ID NO: 4
X¹²ARX¹³X¹⁴LEX¹⁵, where X¹² is T or V; X¹³ is H or T: X¹⁴ is L; X¹⁵ is Q or W.

In some embodiments, provided herein are affinity agents comprising a TPR ligand comprising 7 helices in which the 1^{st} helix comprises the sequence SEQ ID NO: 1
X¹RWX²X³, where X¹ is A; X² is E; X³ is F or V;
the 3^{rd} helix comprises the sequence SEQ ID NO: 2
X⁴AWX⁵X⁶LGX⁷, where X⁴ is W; X⁵ is E; X⁶ is H; X⁷ is W;
the 5^{th} helix comprises the sequence SEQ ID NO: 3
X⁸AWX⁹X¹⁰LGX¹¹, where X⁸ is H; X⁹ is F; X¹⁰ is R; X¹¹ is F; and
the 7^{th} helix comprises the sequence SEQ ID NO: 4
X¹²ARX¹³X¹⁴LEX¹⁵, where X¹² is L or Q; X¹³ is A, or E: X¹⁴ is E or Q; X¹⁵ is S or H.

In some embodiments, provided herein are affinity agents comprising a TPR ligand comprising 7 helices in which the 1^{st} helix comprises the sequence SEQ ID NO: 1
X¹RWX²X³, where X¹ is A, D, E, L, Q or V; X² is A, D, E, or W; X³ is A, D, F, I, N, V, W or Y;
the 3^{rd} helix comprises the sequence SEQ ID NO: 2
X⁴AWX⁵X⁶LGX⁷, where X⁴ is W; X⁵ is E or W; X⁶ is D, E or H; X⁷ is L, V or W;
the 5^{th} helix comprises the sequence SEQ ID NO: 3
X⁸AWX⁹X¹⁰LGX¹¹, where X⁸ is H, R, or V; X⁹ is F; X¹⁰ is R; X¹¹ is F or W; and
the 7^{th} helix comprises the sequence SEQ ID NO: 4
X¹²ARX¹³X¹⁴LEX¹⁵, where X¹² is L, or T; X¹³ is A, D, E, or R; X¹⁴ is E, I, Q, or V; X¹⁵ is D, E, G, H, S or N.

In some embodiments, provided herein are affinity agents comprising a TPR ligand comprising 7 helices in which the 1 helix comprises the sequence SEQ ID No: 1
X¹RWX²X³, where X¹ is D, E or S; X² is Y; X³ is D, Y or H;
The 3^{rd} helix comprises the sequence SEQ ID No: 2
X⁴AWX⁵X⁶LGX⁷, where X⁴ is W; X⁵ is W; X⁶ is E; X⁷ is V, T or A;
The 5^{th} helix comprises the sequence SEQ ID No: 3
X⁸AWX⁹X¹⁰LGX¹¹, where X⁸ is V or I; X⁹ is A, F or S; X¹⁰ is R; X¹¹ is H or W;
The 7^{th} helix comprises the sequence SEQ ID No: 4
X¹²ARX¹³X¹⁴LEX¹⁵, where X¹² is A, L or V; X¹³ is A, H, or S; X¹⁴ is A, E, L or N; X¹⁵ is A, N, V or Y.

In some embodiments, the affinity agents bind the CH1 domain of monoclonal antibody molecules and fragments.

In some embodiments provided herein are affinity agents comprising a ligand that bind to antibodies or antibody fragments comprising the sequence SEQ ID No: 153 &&&&&&&X¹RWX²X³&&&&&&&&&&&&&&&&&&&&&&&&&&&&&X⁴AWX⁵X⁶LG X⁷&&&&&&&&&&&&&&&&&&&&&&&&&&X⁸AWX⁹X¹⁰LGX¹&&&&&&&&&&&& &&&&&&&&&&&&&& X¹²ARX¹³X¹⁴LEX¹⁵, where & denotes any amino acid; X¹ is A, D, E, G, N, Q, S or V; X² is A, E, W or Y; X³ is D, G, H, I, N, T, V, W or Y; X⁴ is W; X⁵ is E or W; X⁶ is D, E or H; X⁷ is T, V, W; X⁸AWX⁹X¹⁰LGX¹¹, where X⁸ is A, H, I, T or V; X⁹ is A, F, N, S or R; X¹⁰ is R; X¹¹ is F, I, H or W; X¹²ARX¹³X¹⁴LEX¹⁵, where X¹² is A, L, T or V; X¹³ is A, D, E, H, Q, R, S or Y; X¹⁴ is A, E, I, L, N or Q; X¹⁵ is A, D, E, H, N R, S, V or Y.

In some embodiments, provided herein are affinity agents comprising a ligand that bind to antibodies or antibody fragments comprising the sequence SEQ ID NO: 5 X¹RWX²X³LGAAYAARGDYDRAIEYYQRALELDPNNAX⁴AWX⁵X⁶LGX⁷AYAARGDYDR AIEYYQRALELDPNNAX⁸AWX⁹X¹⁰LGX¹¹AYAARGDYDRAIEYYQRALELDPNNEX¹²AR X¹³X¹⁴LEX¹⁵, where X¹ is Q, D, A, G, E, V, N; X² is E, A, Y, Q; X³ is D, N, G, V, I, T, W, Y; X⁴ is W; X⁵ is W, E; X⁶ is E, D, H; X⁷ is V, W, T; X⁸ is V, H, T, A, I; X⁹ is F, R, S, A, N; X¹⁰ is R; X¹¹ is H, W, F, I; X¹² is L, T, V; X¹³ is E, Y, R, A, D, S, Q; X¹⁴ is Q, L, E, I, A; X¹⁵ is H, D, Y, S, E, or R.

In some embodiments, affinity agents comprise a ligand comprising any one of the amino acid sequences shown in Table 1, e.g., any one of SEQ ID NOs: 6-152.

In some embodiments, provided herein are affinity agents comprising a ligand comprising SEQ ID NOs: 6-152, or an amino acid sequence that differs by no more than three, by no more than two, or by no more than one, substitutions, additions, or deletions.

In some embodiments, an affinity ligand of the disclosure comprises a C-terminal cysteine or lysine, and preferably a cysteine.

Another aspect of the disclosure provides multimers comprising at least two subunits (e.g., at least two affinity ligands of the disclosure). Such plurality can be a homogenous (i.e., a single ligand of the disclosure) or heterogenous (i.e., including two or more different ligands of the disclosure). In other words, in some embodiments, provided herein are affinity agents that comprise multimer polypeptides where the sub-units are not all the same. In embodiments, a multimers can be a dimer, trimer, tetramer, pentamer, hexamer, heptamer, octamer or nonamer.

In further aspects of the disclosure, the affinity ligand or multimer hereof further comprises at least one heterologous agent operably linked to said affinity ligand to thereby form a conjugate or a fusion protein. Examples of heterologous agent include, but are not limited to, one or more small molecule diagnostic or therapeutic agents; peptides tags (as defined herein) a DNA, RNA, or hybrid DNA-RNA molecule; traceable marker; radioactive agent; an antibody; a single chain variable domain; or an immunoglobulin fragment. Such conjugates and fusion proteins can be made by methods known in the art.

Ligand Binding to an Antibody or Antibody Fragment with a CH1 Domain

The characteristics of a ligand or multimer that binds to a target, such as an antibody or a fragment thereof with a CH1 domain can be determined using known or modified assays, bioassays, and/or animal models known in the art for evaluating such activity.

As used herein, terms such as “binding affinity for a target,” “binding to a target, “binding to an antibody or a fragment thereof with a CH1 domain,” and the like refer to a property of a ligand of the disclosure which may be directly measured, for example, through the determination of affinity constants (e.g., the amount of ligand that associates and dissociates at a given antigen concentration). Several methods are available to characterize such molecular interactions, for example, competition analysis, equilibrium analysis and microcalorimetric analysis, and real-time interaction analysis based on surface plasmon resonance interaction (for example using a BIACORE instrument). These methods are well-known to those of skill in the art and are discussed in publications such as Neri D et al. (1996) Tibtech 14:465-470 and Jansson M et al. (1997) J Biol Chem 272:8189-8197.

Affinity requirements for a given ligand binding event are contingent on a variety of factors including, but not limited to the composition and complexity of the binding matrix, the valency and density of both the ligand and target molecules, and the functional application of the ligand. In some embodiments, a ligand of the disclosure binds an antibody or a fragment thereof with a CH1 domain with a dissociation constant (KD) of less than or equal to 5×10⁻³ M, 10⁻³ M, 5×10⁻⁴ M, 10⁻⁴ M, 5×10⁻⁵ M, or 10⁻⁵ M. In some embodiments, a ligand binds a target of interest with a KD of less than or equal to 5×10⁻⁶ M, 10⁻⁶ M, 5×10⁻⁷ M, 10⁻⁷ M, 5×10⁻⁸ M, or 10⁻⁸ M. In some embodiments, a ligand binds a target of interest with a KD less than or equal to 5×10⁻⁹ M, 10⁻⁹ M, 5×10⁻¹⁰ M, 10⁻¹⁰ M, 5×10⁻¹ M, 10⁻¹¹ M, 5×10⁻² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In some embodiments, a ligand generated by methods disclosed herein has a dissociation constant of from about 10⁻⁴ M to about 10⁻⁵ M, from about 10⁻⁵ M to about 10⁻⁶ M, from about 10⁻⁶ M to about 10⁻⁷ M, from about 10⁻⁷ M to about 10⁻⁸ M, from about 10⁻⁸ M to about 10⁻⁹ M, from about 10⁻⁹ M to about 10⁻¹⁰ M, from about 10⁻¹⁰ M to about 10⁻¹¹ M, or from about 10⁻¹¹ M to about 10⁻¹² M.

Binding experiments to determine K_{D} and off-rates can be performed in a number of conditions. The buffers in which to make these solutions can readily be determined by one of skill in the art, and depend largely on the desired pH of the final solution. Low pH solutions (<pH 5.5) can be made, for example, in citrate buffer, glycine-HCl buffer, or in succinic acid buffer. High pH solutions can be made, for example, in Tris-HCl, phosphate buffers, or sodium bicarbonate buffers. A number of conditions may be used to determine K_{D} and off-rates for the purpose of determining, for example, optimal pH and/or salt concentrations.

In some embodiments, a ligand or multimer of the disclosure specifically binds an antibody or a fragment thereof with a CH1 domain with a koff ranging from 0.1 to 10⁻⁷ sec⁻¹, 10⁻² to 10⁻⁷ sec⁻¹, or 0.5×10⁻² to 10⁻⁷ sec⁻¹. In some embodiments, a ligand binds a target of interest with an off rate (k_{off}) of less than 5×10⁻² sec⁻¹, 10⁻² sec⁻¹, 5×10⁻³ sec⁻¹, or 10³ sec⁻¹. In some embodiments a ligand binds a target of interest with an off rate (k_{off}) of less than 5×10⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹, or 10⁻⁷ sec⁻¹.

In some embodiments, a ligand or multimer specifically binds an antibody or a fragment thereof with a CH1 domain with a kₒₙ ranging from about 10³ to 10⁷ M⁻¹ sec⁻¹, 10³ to 10⁶ M⁻¹ sec⁻¹, or 10³ to 10⁵ M⁻¹ sec⁻¹. In some embodiments, a ligand (e.g., a ligand fusion protein) binds the target of interest with an on rate (kₒₙ) of greater than 10³ M⁻¹ sec⁻¹, 5×10³ M⁻¹ sec⁻¹, 10⁴ M⁻¹ sec⁻¹, or 5×10⁴ M⁻¹ sec⁻¹. In an additional embodiment, a ligand, binds a target of interest with a kₒₙ of greater than 10⁵ M⁻¹ sec⁻¹, 5×10⁵ M⁻¹ sec⁻¹, 10⁶ M⁻¹ sec⁻¹, 5×10⁶ M⁻¹ sec⁻¹, or 10⁷ M⁻¹ sec⁻¹.

### Linkers

The terms “linker” and “spacer” are used interchangeably herein to refer to a peptide or other chemical linkage that functions to link otherwise independent functional domains. In some embodiments, a linker is located between a ligand and another polypeptide component containing an otherwise independent functional domain. Suitable linkers for coupling two or more linked ligands may generally be any linker used in the art to link peptides, proteins or other organic molecules. In some embodiments, such a linker is suitable for constructing proteins or polypeptides that are intended for pharmaceutical use.

Suitable linkers for operably linking a ligand and an additional component of a ligand fusion protein in a single-chain amino acid sequence include but are not limited to, polypeptide linkers such as glycine linkers, serine linkers, mixed glycine/serine linkers, glycine- and serine-rich linkers or linkers composed of largely polar polypeptide fragments.

In some embodiments, a linker comprises a majority of amino acids selected from glycine, alanine, proline, asparagine, glutamine, and lysine. In some embodiments, a linker comprises a majority of amino acids selected from glycine, alanine, proline, asparagine, aspartic acid, threonine, glutamine, and lysine. In some embodiments, a ligand linker is made up of a majority of amino acids that are sterically unhindered. In some embodiments, a linker comprises a majority of amino acids selected from glycine, serine, and/or alanine. In some embodiments, a linker is selected from polyglycines (such as (Gly)₅, and (Gly)₈, poly(Gly-Ala), and polyalanines.

Linkers can be of any size or composition so long as they are able to operably link a ligand in a manner that permits the ligand to bind a target of interest. In some embodiments, linkers are from about 1 to 50 amino acids, from about 1 to 20 amino acids, from about 1 to 15 amino acids, from about 1 to 10 amino acids, from about 1 to 5 amino acids, from about 2 to 20 amino acids, from about 2 to 15 amino acids, from about 2 to 10 amino acids, or from about 2 to 5 amino acids. It should be clear that the length, the degree of flexibility and/or other properties of the linker(s) may influence certain properties of a ligand for use in an affinity agent, such as affinity, specificity or avidity for a target of interest, or for one or more other target proteins of interest, or for proteins not of interest (i.e., non-target proteins). In some embodiments, two or more linkers are utilized. In some embodiments, two or more linkers are the same. In some embodiments, two or more linkers are different.

In some embodiments, a linker is a non-peptide linker such as an alkyl linker, or a PEG linker. For example, alkyl linkers such as —NH—(CH₂)ₛ—C(0)-, wherein s=2-20 can be used. These alkyl linkers may further be substituted by any non-sterically hindering group such as lower alkyl e.g., C₁-C₆) lower acyl, halogen (e.g., Cl, I, Br, F), CN, NH₂, phenyl, etc. An exemplary non-peptide linker is a PEG linker. In some embodiments, a PEG linker has a molecular weight of from about 100 to 5000 kDa, or from about 100 to 500 kDa.

Linkers can be evaluated using techniques described herein and/or otherwise known in the art. In some embodiments, linkers do not alter (e.g., do not disrupt) the ability of a ligand to bind a target molecule.

### Affinity Agents Comprising Conjugated Ligands: Affinity Separation Matrices

Ligands or multimers that promote specific binding to targets of interest can be chemically conjugated to a variety of surfaces used in chromatography, e.g., beads, resins, gels, membrane, monoliths, etc. to prepare an affinity agent. Affinity agents of the disclosure are particularly useful for an antibody or a fragment thereof with a CH1 domain purification and manufacturing applications.

In some embodiments, a ligand of the disclosure (e.g., a ligand fusion protein) contains at least one reactive residue. Reactive residues are useful, for example, as sites for the attachment of conjugates such as chemotherapeutic drugs or diagnostic agents. Exemplary reactive amino acid residues include lysine or cysteine, for example. A reactive residue can be added to a ligand at either end, or within the ligand sequence and/or can be substituted for another amino acid within the ligand sequence. A suitable reactive residue (e.g., lysine, cysteine, etc.) can also be located within the sequence of an identified ligand without need for addition or substitution.

### Attachment to a Solid Surface

“Solid surface,” “support,” or “matrix” are used interchangeably herein and refer to, without limitation, any column (or column material), bead, test tube, microtiter dish, solid particle (for example, agarose or sepharose), microchip (for example, silicon, silicon-glass, or gold chip), or membrane (synthetic (e.g. a filter) or biological (e.g. liposome or vesicle) in origin to which a ligand or multimer of the disclosure may be attached (i.e., coupled, linked, or adhered), either directly or indirectly (for example, through other binding partner intermediates such as a linker), or in which a ligand or multimer may be embedded (for example, through a receptor or channel). Reagents and techniques for attaching polypeptides to solid supports are well-known in the art, e.g., carbamate coupling. Suitable solid supports include, but are not limited to, a chromatographic resin or matrix (e.g., SEPHAROSE-4 FF agarose beads), the wall or floor of a well in a plastic microtiter dish, a silica-based biochip, polyacrylamide, agarose, silica, nitrocellulose, paper, plastic, nylon, metal, and combinations thereof. Ligands and other compositions may be attached on a support material by a non-covalent association or by covalent bonding, using reagents and techniques known in the art. In some embodiments, a ligand is coupled to a chromatography material using a linker.

In one aspect, the disclosure provides an affinity agent (affinity separation matrix) comprised of a ligand or multimer as described above coupled to an insoluble support. Such a support may be one or more particles, such as beads; membranes; filters; capillaries; monoliths; and any other format commonly used in chromatography. In an advantageous embodiment of the affinity separation matrix, the support is comprised of substantially spherical particles, also known as beads. Suitable particle sizes may be in the diameter range of 5-500 μm, such as 10⁻¹⁰⁰ μm, e.g., 20-80 μm. In an alternative embodiment, the support is a membrane. To obtain high adsorption capacities, the support is preferably porous, and ligands may be coupled to the external surfaces as well as to the pore surfaces. In an advantageous embodiment of this aspect, the support is porous.

In another aspect, the disclosure relates to a method of preparing a chromatography affinity agent, which method comprises providing ligands as described above, and coupling the ligands to a support. Coupling may be carried out via a nitrogen or sulfur atom of the ligand for example. The ligands may be coupled to the support directly or indirectly via a spacer element to provide an appropriate distance between the support surface and the ligand. Methods for immobilization of protein ligands to porous or non-porous surfaces are well known in this field.

### Production of Ligands

The production of ligands and multimers, useful in practicing several embodiments of the disclosure, may be carried out using a variety of standard techniques for chemical synthesis, semi-synthetic methods, and recombinant DNA methodologies known in the art. Also provided are methods for producing a ligand or multimer, individually or as part of multi-domain fusion protein, as soluble agents and cell associated proteins. In some embodiments, the overall production scheme for a ligand or multimer comprises obtaining a reference protein scaffold and identifying a plurality of residues within the scaffold for modification. Depending on the embodiment, the reference scaffold may comprise a protein structure with one or more alpha-helical regions, or other tertiary structure. Once identified, any of a plurality of residues can be modified, for example by substitution of one or more amino acids. In some embodiments, one or more conservative substitutions are made. In some embodiments, one or more non-conservative substitutions are made. In some embodiments a natural amino acid (e.g., one of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine) is substituted into a reference scaffold at targeted positions for modification. In some embodiments, modifications do not include substituting in either a cysteine or a proline. After modifications have been made at identified positions desired in a particular embodiment, the resulting modified polypeptides (e.g., candidate ligands) can be recombinantly expressed, for example in a plasmid, bacteria, phage, or other vector (e.g., to increase the number of each of the modified polypeptides). The modified polypeptides can then be purified and screened to identify those modified polypeptides that have specific binding to a particular target of interest, e.g., an antibody or a fragment thereof with a CH1 domain. Modified polypeptides may show enhanced binding specificity for an antibody or a fragment thereof with a CH1 domain as compared to a reference scaffold, or may exhibit little or no binding to a given target of interest (or to a non-target protein). In some embodiments, depending on the target of interest, the reference scaffold may show some interaction (e.g., nonspecific interaction) with the target of interest, while certain modified polypeptides will exhibit at least about two-fold, at least about five-fold, at least about tenfold, at least about 20-fold, at least about 50-fold, or at least about 100-fold (or more) increased binding specificity for the target of interest. Additional details regarding production, selection, and isolation of ligand are provided in more detail below.

### Recombinant Expression of Ligands

In some embodiments, a ligand such as a ligand fusion protein is “recombinantly produced,” (i.e., produced using recombinant DNA technology). Exemplary recombinant methods available for synthesizing ligand fusion proteins, include, but are not limited to polymerase chain reaction (PCR) based synthesis, concatemerization, seamless cloning, and recursive directional ligation (RDL) (see, e.g., Meyer et al., Biomacromolecules 3:357-367 (2002), Kurihara et al., Biotechnol. Lett. 27:665-670 (2005), Haider et al., Mol. Pharm. 2:139-150 (2005); and McMillan et al., Macromolecules 32(11):3643-3646 (1999).

In another aspect, nucleic acids comprising a polynucleotide sequence encoding a ligand or multimer according to the embodiments disclosed above are also provided. Thus, the disclosure encompasses all forms of the present nucleic acid sequence such as RNA and DNA encoding the polypeptide (ligand) or multimer. The disclosure provides vectors, such as plasmids, which in addition to the coding sequence comprise the required signal sequences for expression of the polypeptide or multimer according the disclosure. Such polynucleotides optionally further comprise one or more expression control elements. For example, a polynucleotide can comprise one or more promoters or transcriptional enhancers, ribosomal binding sites, transcription termination signals, and polyadenylation signals, as expression control elements. A polynucleotide can be inserted within any suitable vector, which can be contained within any suitable host cell for expression. In one embodiment, the vector comprises nucleic acid encoding a multimer according to the disclosure, wherein the separate nucleic acids encoding each unit may have homologous or heterologous DNA sequences.

The expression of nucleic acids encoding ligands and multimers is typically achieved by operably linking a nucleic acid encoding the ligand to a promoter in an expression vector. Typical expression vectors contain transcription and translation terminators, initiation sequences, and promoters useful for regulation of the expression of the desired nucleic acid sequence. Exemplary promoters useful for expression in E. coli include, for example, the T7 promoter.

Methods known in the art can be used to construct expression vectors containing the nucleic acid sequence encoding a ligand along with appropriate transcriptional/translational control signals. These methods include, but are not limited to in vitro recombinant DNA techniques, synthetic techniques and in vivo recombination/genetic recombination. The expression of the polynucleotide can be performed in any suitable expression host known in the art including, but not limited to, bacterial cells, yeast cells, insect cells, plant cells or mammalian cells. In some embodiments, a nucleic acid sequence encoding a ligand is operably linked to a suitable promoter sequence such that the nucleic acid sequence is transcribed and/or translated into ligand in a host.

A variety of host-expression vector systems can be utilized to express a nucleic acid encoding a ligand. Vectors containing the nucleic acids encoding a ligand (e.g., individual ligand subunits or ligand fusions) or portions or fragments thereof, include plasmid vectors, a single and double-stranded phage vectors, as well as single and double-stranded RNA or DNA viral vectors. Phage and viral vectors may also be introduced into host cells in the form of packaged or encapsulated virus using known techniques for infection and transduction. Moreover, viral vectors may be replication competent or alternatively, replication defective. Alternatively, cell-free translation systems may also be used to produce the protein using RNAs derived from the DNA expression constructs (see, e.g., WO86/05807 and WO89/01036; and U.S. Pat. No. 5,122,464).

Generally, any type of cell or cultured cell line can be used to express a ligand or multimer provided herein. In some embodiments a background cell line used to generate an engineered host cell is a phage, a bacterial cell, a yeast cell or a mammalian cell. A variety of host-expression vector systems may be used to express the coding sequence a ligand fusion protein. Mammalian cells can be used as host cell systems transfected with recombinant plasmid DNA or cosmid DNA expression vectors containing the coding sequence of the target of interest and the coding sequence of the fusion polypeptide. The cells can be primary isolates from organisms, cultures, or cell lines of transformed or transgenic nature.

Suitable host cells include but are not limited to microorganisms such as, bacteria (e.g., E. coli, B. subtilis) transformed with recombinant bacteriophage DNA, plasmid DNA or cosmid DNA expression vectors containing ligand coding sequences; yeast (e.g., Saccharomyces, Pichia) transformed with recombinant yeast expression vectors containing ligand coding sequences; insect cell systems infected with recombinant virus expression vectors (e.g., Baculovirus) containing ligand coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing ligand coding sequences.

Prokaryotes useful as host cells in producing a ligand include gram negative or gram positive organisms such as, E. coli and B. subtilis. Expression vectors for use in prokaryotic host cells generally contain one or more phenotypic selectable marker genes (e.g., genes encoding proteins that confer antibiotic resistance or that supply an autotrophic requirement). Examples of useful prokaryotic host expression vectors include the pKK223-3 (Pharmacia, Uppsala, Sweden), pGEM1 (Promega, Wis., USA), pET (Novagen, Wis., USA) and pRSET (Invitrogen, Calif., USA) series of vectors (see, e.g., Studier, J. Mol. Biol. 219:37 (1991) and Schoepfer, Gene 124:83 (1993)). Exemplary promoter sequences frequently used in prokaryotic host cell expression vectors include T7, (Rosenberg et al., Gene 56:125-135 (1987)), beta-lactamase (penicillinase), lactose promoter system (Chang et al., Nature 275:615 (1978)); and Goeddel et al., Nature 281:544 (1979)), tryptophan (trp) promoter system (Goeddel et al., Nucl. Acids Res. 8:4057, (1980)), and tac promoter (Sambrook et al., 1990, Molecular Cloning, A Laboratory Manual, 2d Ed., Cold Spring Harbor Laboratory, Cold Spring Harbor, N.Y.).

In some embodiments, a eukaryotic host cell system is used, including yeast cells transformed with recombinant yeast expression vectors containing the coding sequence of a ligand. Exemplary yeast that can be used to produce compositions of the disclosure, include yeast from the genus Saccharomyces, Pichia, Actinomycetes and Kluyveromyces. Yeast vectors typically contain an origin of replication sequence from a 2mu yeast plasmid, an autonomously replicating sequence (ARS), a promoter region, sequences for polyadenylation, sequences for transcription termination, and a selectable marker gene. Examples of promoter sequences in yeast expression constructs include promoters from metallothionein, 3-phosphoglycerate kinase (Hitzeman, J. Biol. Chem. 255:2073 (1980)) and other glycolytic enzymes, such as, enolase, glyceraldehyde-3-phosphate dehydrogenase, hexokinase, pyruvate decarboxylase, phosphofructokinase, glucose-6-phosphate isomerase, 3-phospho glycerate mutase, pyruvate kinase, triosephosphate isomerase, phosphoglucose isomerase, and glucokinase. Additional suitable vectors and promoters for use in yeast expression as well as yeast transformation protocols are known in the art. See, e.g., Fleer, Gene 107:285-195 (1991) and Hinnen, PNAS 75:1929 (1978).

Insect and plant host cell culture systems are also useful for producing the compositions of the disclosure. Such host cell systems include for example, insect cell systems infected with recombinant virus expression vectors (e.g., baculovirus) containing the coding sequence of a ligand; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the coding sequence of a ligand, including, but not limited to, the expression systems taught in U.S. Pat. No. 6,815,184; U.S. Publ. Nos. 60/365,769, and 60/368,047; and WO2004/057002, WO2004/024927, and WO2003/078614.

In some embodiments, host cell systems may be used, including animal cell systems infected with recombinant virus expression vectors (e.g., adenoviruses, retroviruses, adeno-associated viruses, herpes viruses, lentiviruses) including cell lines engineered to contain multiple copies of the DNA encoding a ligand either stably amplified (CHO/dhfr) or unstably amplified in double-minute chromosomes (e.g., murine cell lines). In some embodiments, a vector comprising a polynucleotide(s) encoding a ligand is polycistronic. Exemplary mammalian cells useful for producing these compositions include 293 cells (e.g., 293T and 293F), CHO cells, BHK cells, NS0 cells, SP2/0 cells, YO myeloma cells, P3X⁶³ mouse myeloma cells, PER cells, PER.C6 (Crucell, Netherlands) cells VERY, Hela cells, COS cells, MDCK cells, 3T3 cells, W138 cells, BT483 cells, Hs578T cells, HTB2 cells, BT20 cells, T47D cells, CRL7030 cells, HsS78Bst cells, hybridoma cells, and other mammalian cells. Additional exemplary mammalian host cells that are useful in practicing the embodiments of the disclosure include but are not limited, to T cells. Exemplary expression systems and selection methods are known in the art and, including those described in the following references and references cited therein: Borth et al., Biotechnol. Bioen. 71(4):266-73 (2000), in Werner et al., Arzneimittelforschung/Drug Res. 48(8):870-80 (1998), Andersen et al., Curr. Op. Biotechnol. 13:117-123 (2002), Chadd et al., Curr. Op, Biotechnol. 12:188-194 (2001), and Giddings, Curr. Op. Biotechnol. 12:450-454 (2001). Additional examples of expression systems and selection methods are described in Logan et al., PNAS 81:355-359 (1984), Birtner et al. Methods Enzymol. 153:51-544 (1987)). Transcriptional and translational control sequences for mammalian host cell expression vectors are frequently derived from viral genomes. Commonly used promoter sequences and enhancer sequences in mammalian expression vectors include, sequences derived from Polyoma virus, Adenovirus 2, Simian Virus 40 (SV40), and human cytomegalovirus (CMV). Exemplary commercially available expression vectors for use in mammalian host cells include pCEP4 (Invitrogen) and pcDNA3 (Invitrogen).

Physical methods for introducing a nucleic acid into a host cell (e.g., a mammalian host cell) include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian (e.g., human) cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Methods for introducing a DNA and RNA polynucleotides of interest into a host cell include electroporation of cells, in which an electrical field is applied to cells in order to increase the permeability of the cell membrane, allowing chemicals, drugs, or polynucleotides to be introduced into the cell. Ligand containing DNA or RNA constructs may be introduced into mammalian or prokaryotic cells using electroporation.

In some embodiments, electroporation of cells results in the expression of a ligand-CAR on the surface of T cells, NK cells, NKT cells. Such expression may be transient or stable over the life of the cell. Electroporation may be accomplished with methods known in the art including MaxCyte GT® and STX® Transfection Systems (MaxCyte, Gaithersburg, MD, USA).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo). In some embodiments, the nucleic acid is associated with a lipid. A nucleic acid associated with a lipid can be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they can be present in a bilayer structure, as micelles, or with a “collapsed” structure. They can also simply be interspersed in a solution, possibly forming aggregates that are not uniform in size or shape. Lipids are fatty substances which can be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristoyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristoyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5:505-510 (1991)). However, compositions that have different structures in solution than the normal vesicular structure are also encompassed. For example, the lipids can assume a micellar structure or merely exist as non-uniform aggregates of lipid molecules. Also contemplated are lipofectamine-nucleic acid complexes.

Regardless of the method used to introduce exogenous nucleic acids into a host cell, the presence of the recombinant nucleic acid sequence in the host cell can routinely be confirmed through a variety of assays known in the art. Such assays include, for example, “molecular biological” assays, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Reporter genes are used for identifying potentially transfected cells and for evaluating the functionality of regulatory sequences. In general, a reporter gene is a gene that is not present in or expressed by the recipient organism, tissue, or cell and that encodes a polypeptide whose expression is manifested by some easily detectable property, e.g., enzymatic activity. Expression of the reporter gene is assayed at a suitable time after the DNA has been introduced into the recipient cells. Suitable reporter genes include, but are not limited to, genes encoding luciferase, beta-galactosidase, chloramphenicol acetyl transferase, secreted alkaline phosphatase, or the green fluorescent protein gene (e.g., Ui-Tei et al., FEBS Lett. 479:79-82 (2000)). Suitable expression systems are known in the art and can be prepared using known techniques or obtained commercially. In general, the construct with the minimal 5′ flanking region showing the highest level of expression of reporter gene is identified as the promoter. Such promoter regions can routinely be linked to a reporter gene and used to evaluate agents for the ability to modulate promoter-driven transcription.

A number of selection systems can be used in mammalian host-vector expression systems, including, but not limited to, the herpes simplex virus thymidine kinase, hypoxanthine-guanine phosphoribosyltransferase and adenine phosphoribosyltransferase (Lowy et al., Cell 22:817 (1980)) genes. Additionally, antimetabolite resistance can be used as the basis of selection for e.g., dhfr, gpt, neo, hygro, trpB, hisD, ODC (ornithine decarboxylase), and the glutamine synthase system.

In some embodiments, the initiator N-terminal methionine is included at the NH-terminus of the ligand. In many instances the ligand is isolated without the N-terminal methionine residue, which is presumed to be cleaved during expression. In many instances a mixture is obtained with only a proportion of the purified ligand contains the N-terminal methionine. It is obvious to those skilled in the art that the presence or absence of the N-terminal methionine does not affect the functionality of the ligands and affinity agents described herein.

### Ligand Purification

Once a ligand or a ligand fusion protein or multimer has been produced by recombinant expression, it can be purified by methods known in the art for purification of a recombinant protein, for example, by chromatography (e.g., ion exchange, affinity, and sizing column chromatography), centrifugation, differential solubility, or by any other standard technique for the purification of proteins. In some embodiments, a ligand is optionally fused to heterologous polypeptide sequences specifically disclosed herein or otherwise known in the art to facilitate purification. In some embodiments, ligands (e.g., antibodies and other affinity matrices) for ligand affinity columns for affinity purification and that optionally, the ligand or other components of the ligand fusion composition that are bound by these ligands are removed from the composition prior to final preparation of the ligand using techniques known in the art.

### Chemical Synthesis of Ligand

In addition to recombinant methods, ligand production may also be carried out using organic chemical synthesis of the desired polypeptide using a variety of liquid and solid phase chemical processes known in the art. Various automatic synthesizers are commercially available and can be used in accordance with known protocols. See, for example, Tam et al., J. Am. Chem. Soc., 105:6442 (1983); Merrifield, Science, 232:341-347 (1986); Barany and Merrifield, The Peptides, Gross and Meienhofer, eds, Academic Press, New York, 1-284; Barany et al., Int. J. Pep. Protein Res., 30:705 739 (1987); Kelley et al. in Genetic Engineering Principles and Methods, Setlow, J. K., ed. Plenum Press, NY. 1990, vol. 12, pp. 1-19; Stewart et al., Solid-Phase Peptide Synthesis, W.H. Freeman Co., San Francisco, 1989. One advantage of these methodologies is that they allow for the incorporation of non-natural amino acid residues into the sequence of the ligand.

The ligands and multimers that are used in the methods of the disclosure may be modified during or after synthesis or translation, e.g., by glycosylation, acetylation, benzylation, phosphorylation, amidation, pegylation, formylation, derivatization by known protecting/blocking groups, proteolytic cleavage, linkage to an antibody molecule, hydroxylation, iodination, methylation, myristoylation, oxidation, pegylation, proteolytic processing, phosphorylation, prenylation, racemization, selenoylation, sulfation, ubiquitination, etc. (See, e.g., Creighton, Proteins: Structures and Molecular Properties, 2d Ed. (W.H. Freeman and Co., N.Y., 1992); Postranslational Covalent Modification of Proteins, Johnson, ed. (Academic Press, New York, 1983), pp. 1-12; Seifter, Meth. Enzymol., 182:626-646 (1990); Rattan, Ann. NY Acad. Sci., 663:48-62 (1992).) In some embodiments, the peptides are acetylated at the N-terminus and/or amidated at the C-terminus.

Any of numerous chemical modifications may be carried out by known techniques, including, but not limited to acetylation, formylation, etc. Additionally, derivatives may contain one or more non-classical amino acids.

In some embodiments, cyclization, or macrocyclization of the peptide backbone is achieved by sidechain-to-sidechain linkage formation. Methods for achieving this are well known in the art and may involve natural as well as unnatural amino acids. Approaches includes disulfide formation, lanthionine formation or thiol alkylations (e.g. Michael addition), amidation between amino and carboxylate sidechains, click chemistry (e.g. azide-alkyne condensation), peptide stapling, ring closing metathesis and the use of enzymes.

### Affinity Agents for Purification

In purification based on affinity chromatography, a target of interest (e.g. protein or molecule) is selectively isolated according to its ability to specifically and reversibly bind to a ligand that has typically been covalently coupled to a chromatographic matrix. The affinity ligands of the disclosure can be used as reagents for affinity purification of an antibody or a fragment thereof with a CH1 domain from clarified cell culture fluids (CCCF), or natural sources such as biological samples (e.g., serum), for example, from synthetic or recombinant production of such antibody fragments, or classic enzyme digestion of whole antibodies.

In some embodiments, a ligand or multimer that specifically binds an antibody or a fragment thereof with a CH1 domain is immobilized on beads, such as agarose beads, to form an affinity separation matrix, and then used to affinity purify the target.

Methods of covalently coupling proteins to a surface are known by those of skill in the art, and peptide tags that can be used to attach a ligand to a solid surface are known to those of skill in the art. Further, ligands can be attached (i.e., coupled, linked, or adhered) to a solid surface using any reagents or techniques known in the art. In some embodiments, a solid support comprises beads, glass, slides, chips and/or gelatin. Thus, a series of ligands can be used to make an array on a solid surface using techniques known in the art. For example, U.S. Publ. No. 2004/0009530, which is incorporated herein by reference, discloses methods for preparing arrays.

In some embodiments, a ligand or multimer is used to isolate an antibody or a fragment thereof with a CH1 domain by affinity chromatography. In some embodiments, a ligand or multimer is immobilized on a solid support. The ligand or multimer can be immobilized on the solid support using techniques and reagents described herein or otherwise known in the art. Suitable solid supports are described herein or otherwise known in the art and in specific embodiments are suitable for packing a chromatography column. The affinity agent can be packed in columns of various sizes and operated at various linear velocities or immobilized affinity ligand can be contacted with a solution under conditions favorable to form a complex between the ligand and an antibody or a fragment thereof with a CH1 domain. Non-binding materials can be washed away. Suitable wash conditions can readily be determined by one of skill in the art. Examples of suitable wash conditions are described in Shukla and Hinckley, Biotechnol Prog. 2008 September-October; 24(5):1115-21. doi: 10.1002/btpr.50.

In some embodiments, chromatography is carried out by mixing a solution containing the target of interest and the ligand, then isolating complexes of the target of interest and ligand, e.g., an antibody or a fragment thereof with a CH1 domain and ligand. For example, a ligand or multimer is immobilized on a solid support such as beads, then separated from a solution along with the an antibody or a fragment thereof with a CH1 domain by filtration. In some embodiments, the ligand or multimer is a fusion protein that contains a peptide tag, such as a poly-His tail or streptavidin binding region, which can be used to isolate the ligand or multimer after complexes have formed using an immobilized metal affinity chromatographic resin or streptavidin-coated substrate. Once separated, the an antibody or a fragment thereof with a CH1 domain can be released from the ligand or multimer under elution conditions and recovered in a purified form.

In some embodiments, a ligand or multimer of the disclosure is coupled to a highly cross-linked agarose base matrix which is useful for bioprocess applications. Attachment of the ligand or multimer to the base matrix may be through a flexible spacer that ensures ligand accessibility and subsequently leads to high binding capacities. The affinity of the ligands and multimers of the disclosure ensures highly specific binding of an antibody or a fragment thereof with a CH1 domain at near neutral pH (pH 6-9), while enabling elution at pH as high as 4.5. Moreover, the ligands of the disclosure are designed for enhanced alkali stability, enabling the repeated use of from 0.1 M to 0.5 M NaOH in cleaning-in-place (CIP) and sanitization applications.

In another aspect, the disclosure provides, a method of isolating an antibody or a fragment thereof with a CH1 domain, wherein a separation matrix as disclosed above is used. In certain embodiments, the method comprises the steps of (a) contacting a liquid sample comprising an antibody or a fragment thereof with a CH1 domain with a separation matrix as disclosed above, (b) washing the separation matrix with a washing liquid, (c) eluting the an antibody or a fragment thereof with a CH1 domain from the separation matrix with an elution liquid, and (d) cleaning the separation matrix with a cleaning liquid, which can alternatively be called a cleaning-in-place (CIP) liquid, e.g. with a contact (incubation) time of at least one minute, e.g., for one to four minutes or more.

Suitable compositions of the liquid sample, the washing liquid and the elution liquid, as well as the general conditions for performing the separation are well known in the art of affinity chromatography. A liquid sample comprising an antibody or a fragment thereof with a CH1 domain may comprise host cell proteins (HCP), such as HEK293T cells for example. The host cell proteins may be desorbed during step (b).

Binding of an antibody or a fragment thereof with a CH1 domain has been demonstrated with buffers at near-neutral pH (6-9) over a wide range of ionic strength (e.g., 100-400 mM NaCl). Conventional buffers, e.g., phosphate, citrate, acetate, Tris, may be used for equilibration and loading.

In some embodiments, a solution or sample containing an antibody or a fragment thereof with a CH1 domain is concentrated, for example by ultrafiltration, prior to contacting the solution with the separation matrix. For example, the antibody or antibody fragment-containing solution, e.g., a clarified cell culture feed, may be concentrated up to 20-fold. Concentration of the antibody or antibody fragments reduces the load time for affinity chromatography. Increase in concentration may also have a positive effect on the binding capacity due to thermodynamic equilibrium effects, which may lead to a lower volume of separation matrix needed for purification. Concentrating the feed of antibody or antibody fragment solution can also lead to a significant gain in the processing time.

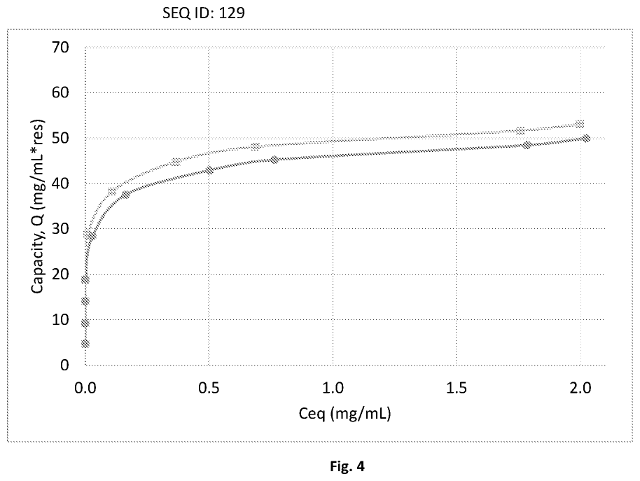

Alternatively, the solution or sample containing an antibody or a fragment thereof with a CH1 domain is a non-concentrated or diluted solution, e.g., a clarified cell culture feed (CCCF). As shown in FIG. 4, the affinity separation matrix of the disclosure is characterized by an ability to process CCCF at high volumetric flow rates, enabling capture from dilute CCCF feed streams.

Elution of antibodies or fragments thereof is generally achieved by lowering the pH, e.g., 2.0-3.0, although higher pH may be used. Optimal conditions for elution of an antibody or a fragment thereof with a CH1 domain can be readily determined by those of skill in this field.

The affinity agents of the disclosure are alkali-tolerant, enabling the use of NaOH up to concentrations of 0.5 M for cleaning. In certain embodiments, a CIP regimen of 0.5 M NaOH exposure for up to 30 to 60 minutes per cycle, for example, ensures consistent chromatographic performance for several cycles, e.g., 15-30 cycles, including up to 70%-90% of the initial binding capacity and low residual DNA and HCP levels, as well as substantially no change in flow capacity.

While some embodiments of the invention have been described by way of illustration, it will be apparent that the invention can be put into practice with many modifications, variations and adaptations, and with the use of numerous equivalents or alternative solutions that are within the scope of persons skilled in the art, without departing from the spirit of the invention or exceeding the scope of the claims.

All publications, patents, and patent applications are herein incorporated by reference in their entirety to the same extent as if each individual publication, patent or patent application was specifically and individually indicated to be incorporated by reference in its entirety.

### Exemplary Numerated Embodiments

1. An affinity agent comprising a ligand comprising a TPR ligand comprising 7 helices in which the 1st helix comprises the sequence SEQ ID NO: 1
   X¹RWX²X³, where X¹ is A, D, E, G, N, Q or V; X² is A, E, W or Y; X³ is D, G, H, I, N, T, V, W or Y;
   the 3^{rd} helix comprises the sequence SEQ ID NO: 2
   X⁴AWX⁵X⁶LGX⁷ where X⁴ is W; X⁵ is E or W; X⁶ is D, E or H; X⁷ is T, V, W;
   the 5^{th} helix comprises the sequence SEQ ID NO: 3
   X⁸AWX⁹X¹⁰LGX¹¹, where X⁸ is A, H, I, T or V; X⁹ is A, F, N, S or R; X¹⁰ is R; X¹¹ is F, I, H or W; and
   the 7^{th} helix comprises the sequence SEQ ID NO: 4
   X¹²ARX¹³X¹⁴LEX¹⁵, where X¹² is A, L, T or V; X¹³ is A, D, E, H, Q, R, S or Y; X¹⁴ is A, E, I, L, N or Q; X¹⁵ is A, D, E, H, N, R, S, V or Y.
2. Affinity agents comprising a TPR ligand comprising 7 helices in which the 1^{st} helix comprises the sequence SEQ ID NO: 1
   X¹RWX²X³, where X¹ is E, Q or V; X² is E or Y; X³ is D, I or N;
   the 3^{rd} helix comprises the sequence SEQ ID NO: 2
   X⁴AWX⁵X⁶LGX⁷, where X⁴ is W; X⁵ is W; X⁶ is E; X⁷ is I or V;
   the 5^{th} helix comprises the sequence SEQ ID NO: 3
   X⁸AWX⁹X¹⁰LGX¹¹, where X⁸ is H, T or V; X⁹ is A, F or S; X¹⁰ is R; X¹¹ is H, I, or W; and
   the 7^{th} helix comprises the sequence SEQ ID NO: 4
   X¹²ARX¹³X¹⁴LEX¹⁵, where X¹² is L, T or V; X¹³ is D, R, or Y: X¹⁴ is A, I or L; X¹⁵ is D, H or Y.
3. Affinity agents comprising a TPR ligand comprising 7 helices in which the 1^{st} helix comprises the sequence SEQ ID NO: 1
   X¹RWX²X³, where X¹ is N; X² is Y; X³ is V or Y;
   the 3^{rd} helix comprises the sequence SEQ ID NO: 2
   X⁴AWX⁵X⁶LGX⁷, where X⁴ is F, or Y; X⁵ is D; X⁶ is E; X⁷ is N or S;
   the 5^{th} helix comprises the sequence SEQ ID NO: 3
   X⁸AWX⁹X¹⁰LGX¹¹, where X⁸ is V; X⁹ is N, or S; X¹⁰ is R; X¹¹ is H, or Q; and
   the 7^{th} helix comprises the sequence SEQ ID NO: 4
   X¹²ARX¹³X¹⁴LEX¹⁵, where X¹² is T or V; X¹³ is H or T: X¹⁴ is L; X¹⁵ is Q or W.
4. Affinity agents comprising a TPR ligand comprising 7 helices in which the 1^{st} helix comprises the sequence SEQ ID NO: 1
   X¹RWX²X³, where X¹ is A; X² is E; X³ is F or V;
   the 3^{rd} helix comprises the sequence SEQ ID NO: 2
   X⁴AWX⁵X⁶LGX⁷, where X⁴ is W; X⁵ is E; X⁶ is H; X⁷ is W;
   the 5^{th} helix comprises the sequence SEQ ID NO: 3
   X⁸AWX⁹X¹⁰LGX¹¹, where X⁸ is H; X⁹ is F; X¹⁰ is R; X¹¹ is F; and
   the 7^{th} helix comprises the sequence SEQ ID NO: 4 X¹²ARX¹³X¹⁴LEX¹⁵, where X¹² is L or Q; X¹³ is A, or E: X¹⁴ is E or Q; X¹⁵ is S or H.
5. Affinity agents comprising a TPR ligand comprising 7 helices in which the 1^{st} helix comprises the sequence SEQ ID NO: 1
   X¹RWX²X³, where X¹ is A, D, E, L, Q or V; X² is A, D, E, or W; X³ is A, D, F, I, N, V, W or Y;
   the 3^{rd} helix comprises the sequence SEQ ID NO: 2
   X⁴AWX⁵X⁶LGX⁷, where X⁴ is W; X⁵ is E or W; X⁶ is D, E or H; X⁷ is L, V or W;
   the 5^{th} helix comprises the sequence SEQ ID NO: 3
   X⁸AWX⁹X¹⁰LGX¹¹, where X⁸ is H, R, or V; X⁹ is F; X¹⁰ is R; X¹¹ is F or W; and
   the 7^{th} helix comprises the sequence SEQ ID NO: 4
   X¹²ARX¹³X¹⁴LEX¹⁵, where X¹² is L, or T; X¹³ is A, D, E, or R; X¹⁴ is E, I, Q, or V;
   X¹⁵ is D, E, G, H, S or N.
6. The affinity agent of embodiment 1 that binds to monoclonal antibodies, antibody fragments and antibody conjugates containing a CH1 domain.
7. An comprising a ligand comprising the sequence SEQ ID NO: 5

X¹RWX²X³LGAAYAARGDYDRAIEYYQRALELDPNNAX⁴AWX⁵X⁶LGX⁷AYA
ARGDYDRAIEYYQRALELDPNNAX⁸AWX⁹X¹⁰LGX¹¹AYAARGDYDRAIEYY
QRALELDPNNEX¹²ARX¹³X¹⁴LEX¹⁵,

where X¹ is A, D, E, G, N, Q or V; X² is A, E, W or Y; X³ is D, G, I, N, T, V, W or Y; X⁴ is W; X⁵ is E or W; X⁶ is D, E or H; X⁷ is T, V, W; X⁸ is A, H, I, T or V; X⁹ is A, F, N, S or R; X¹⁰ is R; X¹¹ is F, I, H or W; X¹² is L, T or V; X¹³ is A, D, E, Q, R, S or Y; X¹⁴ is A, E, I, L or Q; X¹⁵ is D, E, H, R, S or Y.
8. An affinity agent comprising a ligand that binds monoclonal antibodies, antibody fragments and antibody conjugates containing a CH1 domain comprising a ligand comprising at least one sequence of SEQ ID NOs: 6-152 and 154-204, or an amino acid sequence that differs by no more than three, by no more than two, or by no more than one, substitutions, additions, or deletions.
9. The affinity agent of any one of claims 1-8, that comprise multimer polypeptides comprising at least two subunits, each subunit being a polypeptide according to any of embodiments 1-2.
10. The affinity agent of embodiment 9, wherein the sub-units are not all the same.
11. The affinity agent according to any of embodiments 1-10, wherein the ligand is attached to a solid surface.
12. The affinity agent of embodiment 11, wherein the solid surface is a resin or bead.
13. The affinity agent of embodiment 11, wherein the solid surface is a membrane.
14. The affinity agent of embodiment 11, wherein the solid surface is a monolith.
15. The affinity agent of any one of embodiments 11-14, wherein the ligand is conjugated to the solid surface via a linker.
16. An affinity agent of any one of embodiments 1-15 that is used for purification of a monoclonal antibodies, antibody fragments and antibody conjugates containing a CH1 domain.
17. A method of making an affinity agent comprising conjugating a ligand according to any of embodiments 1-14 to a solid surface.

### EXAMPLES

The examples presented herein represent certain embodiments of the present invention. However, it is to be understood that these examples are for illustration purposes only and do not intend, nor should any be construed, to be wholly definitive as to conditions and scope of this invention. The examples were carried out using standard techniques, which are well known and routine to those of skill in the art, except where otherwise described in detail.

### Example 1. Methods

Peptides are synthesized by standard Fmoc solid phase peptide synthesis techniques and purified by preparative reverse phase HPLC. The purity of peptides is assessed by RP-UPLC with both UV and quadrupole time-of-flight mass spectrometric detection.

Recombinant affinity ligands are expressed in E. coli or Pichia pastoris using standard techniques. Ligands can be purified using multi-column chromatography. For His-tagged ligands, immobilized metal affinity chromatography (IMAC) is used as the primary capture step. Biotinylated ligands are generated with the AviTag™ system (Avidity, Aurora, CO). Non-biotinylated ligands bearing the AviTag™ sequence are prepared by omitting exogenous biotin. The purity and identity of recombinant protein ligands is assessed by a combination of SDS-PAGE, RP UPLC, quadrupole time-of-flight mass spectrometry and size exclusion chromatography (SEC); Sephadex S75, Cytiva, Marlborough, MA). In many instances the ligand is isolated without the N-terminal methionine residue, which is presumed to be cleaved during expression.

### Example 2. Exemplary CH1 Domain Affinity Ligands

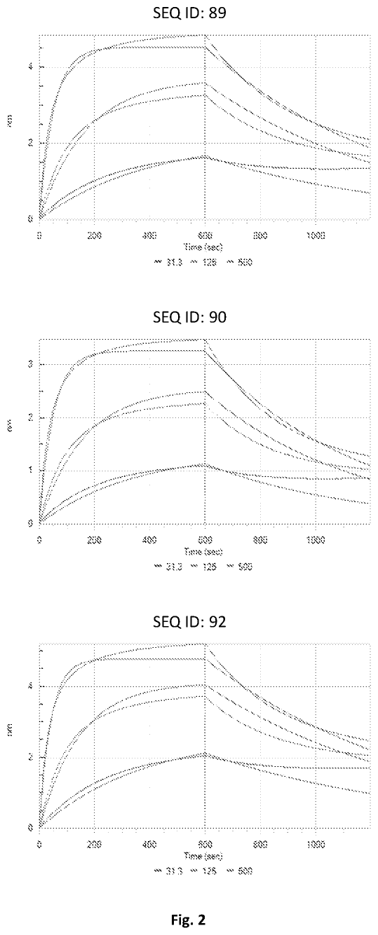

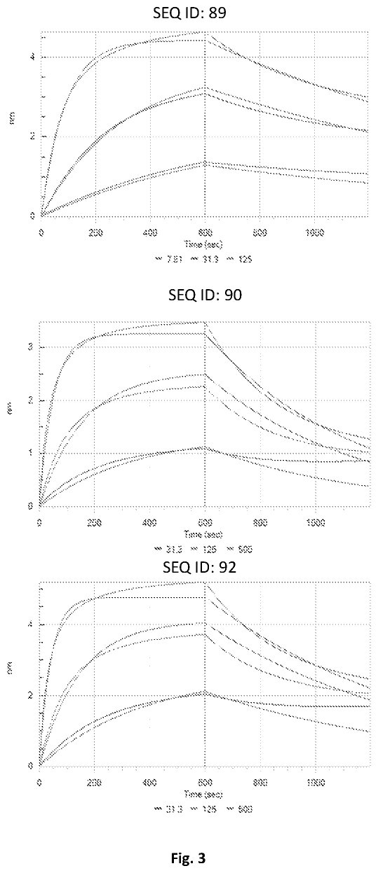

This example demonstrates the binding of biotinylated ligands to Fab fragments or IgG having a CH1 domain using biolayer interferometry (ForteBio, Menlo park, CA). Biotinylated affinity ligands were immobilized on sensors and incubated with solutions containing the Fab fragments or IgG at various concentrations. To demonstrate that the binding site does not occur on either light chain or on a paratope (antigen-binding site), two Fabs with different light chains and paratopes were interrogated for binding. FIG. 2 and FIG. 3 show the sensorgram plots for the binding of affinity ligands 89, 90 and 92 to (a) Herceptin Fab with a kappa light chain (κ) and binding specificity for Her2/neu (from the monoclonal antibody trastuzumab) or (b) Tremfya Fab with a lambda light chain (λ) and binding specificity for IL23 (from the monoclonal antibody guselkumab), respectively.

The binding curves were fit with a 1:1 binding model using the ForteBio software to determine the binding affinities and the resulting data is shown in Table 1. As expected, the affinity ligands agents bound to both Fabs with differing light chains, at binding affinity generally less than 100 nM and in many cases less than 10 nM. Similar experiments Binding to the dimeric human IgG-lambda (Southern Biotech, Birmingham AL) had the highest affinity of all reagents tested.

TABLE 1
Affinity (K_{D}) of selected Ligands for Fabs and Antibodies

| Ligand | Tremfya Fab | Herceptin Fab | IgG |
|---|---|---|---|
| (SEQ ID NO.) | K_{D} (nM) | K_{D} (nM) | K_{D} (nM) |
| 79 | 28 | 67 | <1 |
| 80 | 4 | 7 | <1 |
| 81 | 8 | 31 | <1 |
| 82 | 6 | 23 | <1 |
| 83 | 8 | 22 | <1 |
| 84 | 18 | 32 | <1 |
| 85 | 26 | 53 | <1 |
| 86 | 8 | 28 | <1 |
| 87 | 10 | 84 | <1 |
| 88 | 10 | 32 | <1 |
| 89 | 8 | 41 | <1 |
| 90 | 12 | 51 | <1 |
| 91 | 11 | 46 | <1 |
| 92 | 8 | 28 | <1 |
| 93 | 4 | 15 | <1 |
| 94 | 3 | 14 | <1 |

### Example 3. Alkaline Stability of Affinity Ligands

This example demonstrates the sodium hydroxide stability of the affinity ligands. The indicated affinity ligands were incubated in 0.1 M NaOH for 24 hours and then neutralized. The binding of the NaOH treated ligands was measured as described in Example 2 and compared to untreated ligand. The binding retained was calculated according to the following formula:

% binding retained=(measured response after NaOH treatment)+(measured response of untreated)×100

The data in Table 2 indicate that many of the affinity ligands tested exhibit high stability under tested conditions.

TABLE 2
Percentage (%) binding retained under Alkaline Conditions

| Ligand | % Binding |
|---|---|
| (SEQ ID NO.) | retained |
| 79 | 63 |
| 80 | 66 |
| 81 | 69 |
| 82 | 59 |
| 83 | 55 |
| 84 | 100 |
| 85 | 64 |
| 86 | 56 |
| 87 | 2 |
| 88 | 32 |
| 89 | 53 |
| 90 | 54 |
| 91 | 68 |
| 92 | 53 |
| 93 | 61 |
| 94 | 100 |

### Example 4. Construction of Exemplary Affinity Resins

This example demonstrates the production and characterization of affinity agents comprising ligands identified and described herein. Affinity resins were prepared by conjugating ligands to either bromoacetyl or epoxy activated activated Praesto® Jetted A50 beads (Purolite, King of Prussia, PA) or ABT700 (Agarose Bead Technologies, Madrid, Spain) agarose beads.

Bromoacetyl activated beads were activated with disuccinimidyl carbonate and coupled with excess ethylenediamine, followed by with bromoacetate washing to functionalize the free amine. The affinity ligand was conjugated at room temperature to the bromoacetyl-activated beads via it's carboxy terminal cysteine using EDC activation (EDC is 1-ethyl-3-(3-dimethylaminopropyl)carbodiimide hydrochloride). Epoxy activated beads were either obtained from the vendor or prepared using standard methods well known in the art. Ligands were coupled to epoxy activated beads at 37-40° C. Targeted ligand densities varied from 5-40 g/L. After washing, the beads were deactivated with excess thioglycerol. The actual ligand density for was measured using a subtractive RP-HPLC method according to the following formula:

Actual Ligand Density=(Measured[ligand]in feed−Measured[ligand]in effluent).

### Example 5. Alkaline Stability of Affinity Resins

This example demonstrates the binding characteristics and sodium hydroxide stability of affinity chromatography resins prepared from affinity ligands described herein. Briefly, 10 μL of the affinity resin was aliquoted into replicate wells on a 96-well filter plate and incubated in a neutral pH buffer or in 0.1 M NaOH with 1 M NaCl for 24 hours. After incubation, the affinity resins were extensively washed with a neutral pH buffer and interrogated for binding in a batch format. As outlined in Table 3, following equilibration with PBS, the resins were challenged with IgG at concentrations ranging from 0.5-2 mg/mL for 15 minutes per binding cycle in iterative cycles of binding. After each binding challenge, the multi-well filter plate was centrifuged to separate the unbound IgG from the resin, and the amount of unbound IgG was quantified by absorbance measurement at 280 nm of the effluent.

TABLE 3
Procedure for testing binding characteristics
and Alkaline Stability of Affinity Resins

| | | Volume | Time | # of |
|---|---|---|---|---|
| Step | Buffer | (μL) | (min) | Cycles |
| Equilibrate | PBS | 100 | Vacuum | |
| NaOH treat | 0.1M + 1M NaCl, 24 | 100 | 2+ | 3+ |
| | hours | | | |
| Equilibrate | PBS | 100 | 2 | 5 |
| Load Sample | 0.5 mg/mL mAb | 100 | 15 | 4 |
| Load Sample | 1.0 mg/mL mAb | 100 | 15 | 4 |
| Load Sample | 2.0 mg/mL mAb | 100 | 15 | 4 |

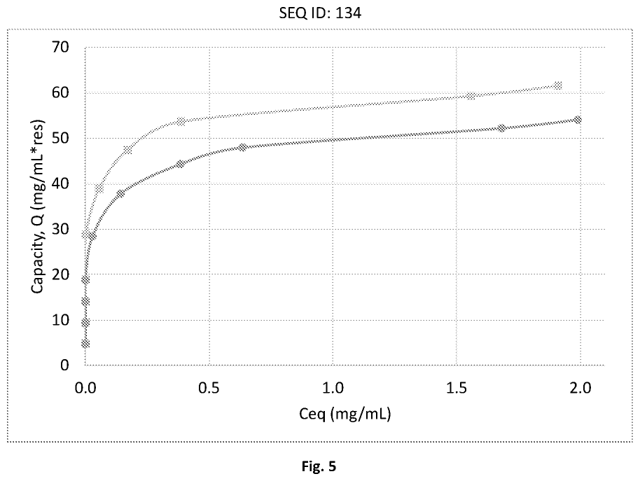

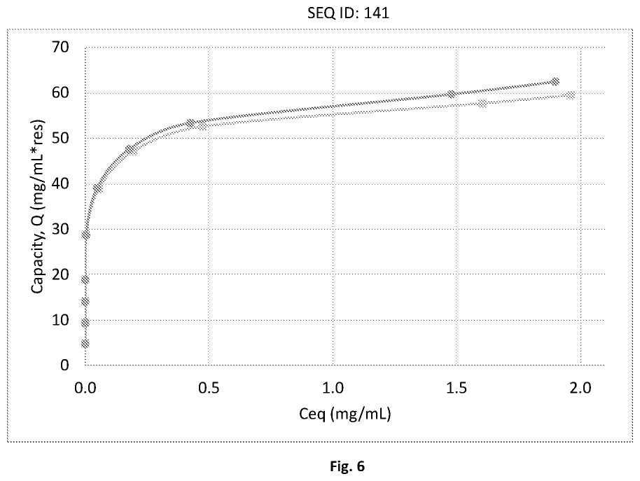

An adsorption isotherm curve was constructed by plotting the concentration of IgG remaining in solution after incubation with resin against amount of IgG bound to resin, at the various challenge conditions. FIGS. 4-6 show the adsorption isotherms for affinity resins with ligands having SEQ ID NOS. 129, 134 and 144, respectively. The results showed that the resin with SEQ ID. 129 retains over 94% binding, that with SEQ ID. 134 retains over 88% binding and that with SEQ ID. 144 retains over 95% binding relative to the initial Qmax after 24 hours exposure to 0.1 M NaOH with 1 M NaCl.

### Example 6. Monoclonal Antibody Purification

A clarified cell culture feed stream (CCCF) from a CHO cell line containing monoclonal antibody (MAbI) at a titer of 2.5 g/L was used loaded onto an affinity resin prepared from the ligand corresponding to SEQ ID No. 129. The resin was packed into an 0.3×10 cm (0.707 mL) column and operated as shown in Table 4.

TABLE 4
Affinity purification of MAb1

| Step | Buffer | CV | V (cm/hr) |
|---|---|---|---|
| Equilibration | PBS | 2 | 150 |
| Load Sample | CCCF (2.5 g/L) | 10.4 | 150 |
| Chase | PBS | 8.5 | 150 |
| Wash | 50 mM Tris, pH 7.5, 500 mM NaCl | 8.5 | 150 |
| Elute | 100 mM Sodium Acetate, pH 3.5 | 5 | 150 |
| CIP | 0.1M NaOH, 1M NaCl | 5.7 | 600 |

Abbreviations for Tables 4-6: CV, column volume; V, velocity; CIP, clean in place.

The fractions were analyzed on a 4-12% Bis-Tris SDS-PAGE gel run under non-reducing conditions. The samples loaded in each lane are (1) SeeBlue Plus2 molecular weight markers, (2) crude feedstream (i.e., the CCCF), (3) 10×-diluted crude feedstream, (4) flow through fraction pool (i.e., chase), (5) wash fraction pool, (6) elution fraction pool (6.4 ug), and (7) strip fraction pool (i.e., CIP pool).

### Example 7. Monoclonal Antibody Purification on CIP Resins

Packed columns were used to purify a monoclonal antibody at a titer of 2.5 mg/mL in a CHO CCCF. Resins were prepared from ligands corresponding to SEQ ID Nos. 129, 134 and 144 as described in Example 4, packed into 0.3×10 cm (0.7 mL) columns and was operated as shown in Table 5.

TABLE 5
Affinity purification of a Monoclonal Antibody

| | | | V |
|---|---|---|---|
| Step | Buffer | CV | (cm/hr) |
| Equilibration | PBS | 2 | 150 |
| Load Sample | CCCF (2.5 g/L) | 11.9 | 150 |
| Chase | PBS | 8.5 | 150 |
| Wash | 50 mM Tris, pH 7.5, 500 mM NaCl | 8.5 | 150 |
| Elute | 100 mM Sodium Acetate, pH 3.5 | 5 | 150 |
| CIP | 0.1M NaOH, 1M NaCl | 5.7 | 600 |
| Re-equilibration | PBS | 14.1 | 600 |

Between each purification cycle, the resins were exposed to the CIP agent for a total of 60 minutes to simulate 4 CIP cycles.

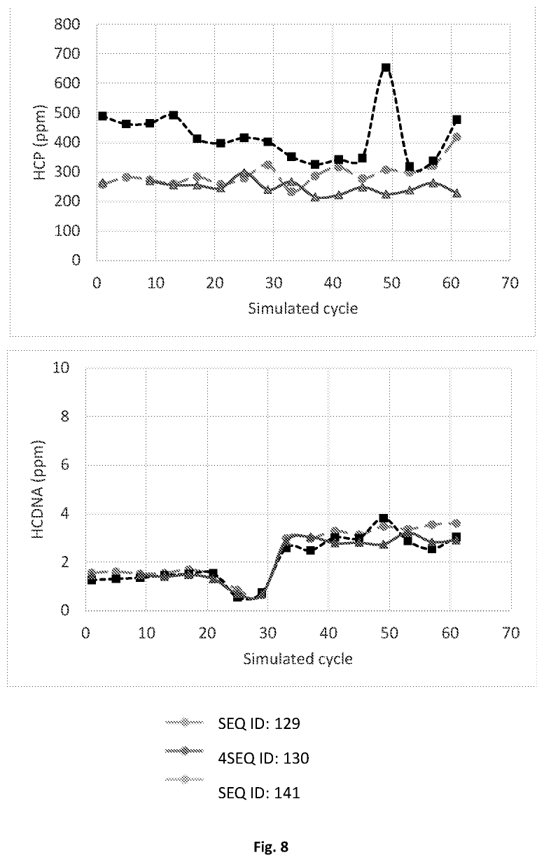

Eluate fractions from each purification cycle were collected and purity was analyzed for residual host cell proteins (HCP) and residual host cell DNA (HCDNA) using the Cygnus™ CHO Host Cell Proteins 3rd Generation assay, and the ThermoFisher Quant-iT™ PicoGreen™ assay, respectively. FIG. 8, top panel, a illustrates the purity of eluate fractions obtained from the affinity resins agents over 60 simulated cycles, with all cycles having HCP less than 700 ppm, and most cycles having HCP less than 300 ppm. Similarly, as shown in FIG. 8, bottom panel, HCDNA was reduced below 5 ppm for the three resins in all cycles. The data confirm that the resin can be cleaned with NaOH and reused for multiple cycles.

### Example 8. Purification Yields

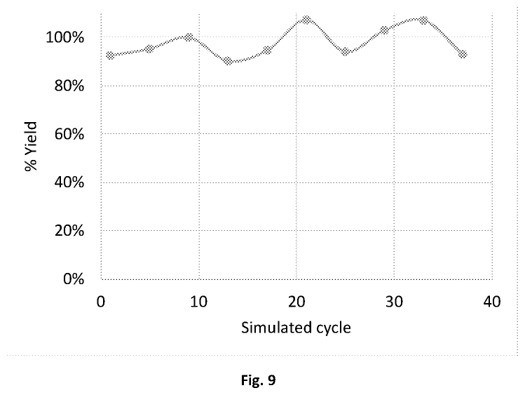

For this study, a purified monoclonal antibody at a concentration of 2.36 mg/mL was loaded onto a 0.3×10 cm (0.7 mL) column packed with an affinity resin with the affinity ligand of SEQ ID. 144. Ten purification cycles were performed as described in Table 6. Between each cycle, the resin was exposed to the CIP agent 0.1 M NaOH, 1 M NaCl for 60 minutes, which is equivalent to 4 simulated CIP cycles. Yields were determined after each of the 10 cycles and plotted as simulated cycles in FIG. 9. The data indicate that the resin can be cleaned with NaOH and reused for multiple cycles while maintaining consistent yield.

TABLE 6
Operating Conditions for Yield Determination

| | | | V |
|---|---|---|---|
| Step | Buffer | CV | (cm/hr) |
| Equilibration | PBS | 2 | 150 |
| Load Sample | Monoclonal antibody in PBS (2.36 | 11.9 | 150 |
| | g/L) | | |
| Chase | PBS | 8.5 | 150 |
| Wash | 50 mM Tris, pH 7.5, 500 mM NaCl | 8.5 | 150 |
| Elute | 100 mM Sodium Acetate, pH 3.5 | 5 | 150 |
| CIP | 0.1M NaOH, 1M NaCl | 5.7 | 600 |
| Re-equilibration | PBS | 14.1 | 600 |

### Example 9. Role of Helix Resins

This example demonstrates that helices 2, 4 and 6 are not critical to the binding. Modifications may be made to helices 2, 4 or 6 and as long as these modifications still enable the TPR to form a stacked helical bundle with helices 1, 3, 5 and 7 forming an inner surface, the ligands maintain binding.

### Example 10. Role of Helix Resins

The role of sequence variation in helix 2 of the 7 helices of these TPR domain-containing ligands was examined by determining the binding affinity of four biotinylated ligands which have identical sequences for helices 1, 3, 5 and 7 but which differ in the sequences for helix 2. Binding affinities were determined as described in Example 2 and the results are shown in Table 7. These results show that modifications made in helix 2 maintain ligand binding.

TABLE 7
Affinity (K_{D}) of Ligands with Helix 2 for Fabs and IgG

| Ligand | Tremfya Fab | Herceptin Fab | IgG |
|---|---|---|---|
| (SEQ ID NO.) | K_{D} (nM) | K_{D} (nM) | K_{D} (nM) |
| 41 | 24 | 15 | 3 |
| 89 | 8 | 41 | <1 |
| 90 | 12 | 51 | <1 |
| 92 | 8 | 28 | <1 |

### Example 11. Binding of Biotinylated Ligands to Monoclonal Antibodies

This example demonstrates the binding of biotinylated ligands to a IgG1 (Praluent®), IgG2 (Ajovy®) and IgG4 (Dupixent®) monoclonal antibodies using biolayer interferometry. Antibodies were obtained from McKesson Supply Manager (Irving, Tx). Biotinylated ligands were immobilized on sensors and incubated with solutions containing monoclonal antibody protein at various concentrations. The binding affinities are shown in the following table.

| | Binding affinity (nM) | | |
|---|---|---|---|
| SEQ ID | Praluent | Ajovy | Dupixent |
| 154 | <1 | 92 | 13 |
| 155 | <1 | 117 | 32 |
| 156 | <1 | 224 | 7 |
| 157 | <1 | 270 | 8 |
| 158 | <1 | 282 | 176 |
| 159 | <1 | 436 | 9 |
| 160 | <1 | 543 | 24 |
| 161 | <1 | 654 | 31 |
| 162 | <1 | 665 | 25 |
| 163 | <1 | >1000 | 13 |
| 164 | <1 | >1000 | 82 |
| 165 | <1 | >1000 | 31 |
| 166 | <1 | >1000 | 120 |
| 167 | <1 | >1000 | 31 |
| 168 | <1 | >1000 | 232 |
| 169 | <1 | >1000 | 31 |
| 170 | <1 | >1000 | 28 |
| 171 | <1 | >1000 | 242 |
| 172 | <1 | >1000 | 215 |
| 173 | <1 | >1000 | 95 |
| 174 | <1 | >1000 | 221 |

### Example 12. Binding of Biotinylated Ligands to mAB and Fab

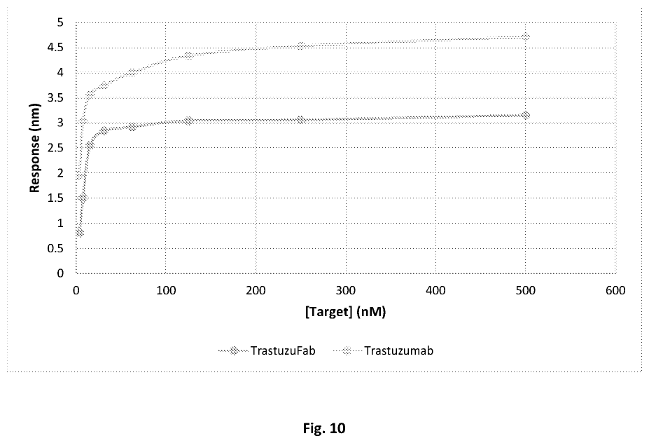

This example demonstrates the binding of biotinylated ligands to full mAb and Fab molecules. Biotinylated ligand corresponding to SEQ ID No: 181 was immobilized on sensors and incubated with solutions containing either Trastuzumab or Trastuzufab protein at various concentrations. It is evident from the dose response curve for binding shown in FIG. 10 that the ligand binds with high affinity to both molecules.

### Example 13. Affinity Resins with High Binding Capacity

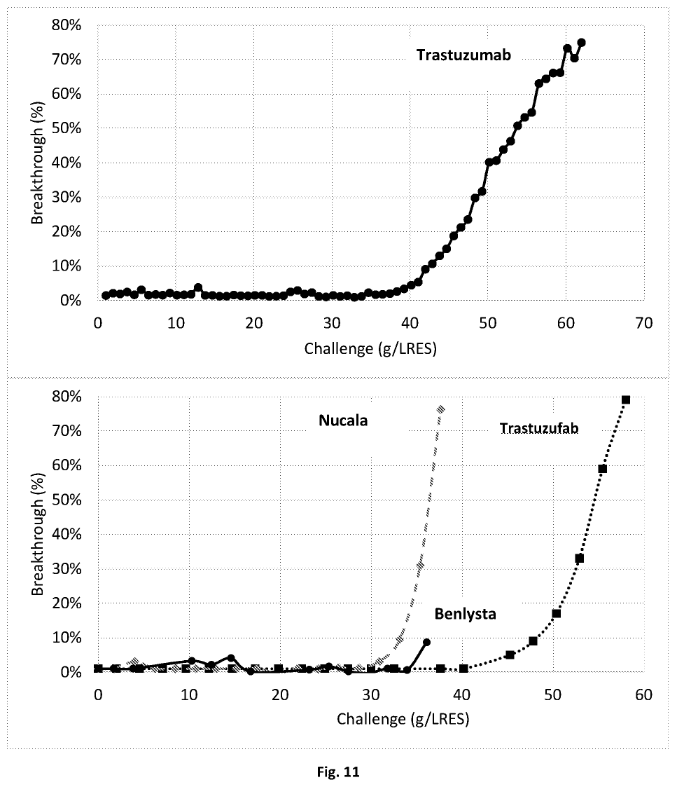

This example demonstrates that affinity resins with high binding capacities can be achieved with ligands of the invention. An affinity resin was prepared with the ligand corresponding to SEQ ID No: 181 conjugated to epoxy activated Jetted A50 agarose beads and packed into a 3×50 mm column. The binding capacity of Trastuzumab, Trastuzufab, Nucala® and Benlysta® was obtained at 4 minute residence time using a challenge concentration of 2 g/L. The breakthrough curves are shown in FIG. 11 and the 10% breakthrough values are listed in the following table.

| Analyte | DBC @ 10% Breakthrough |
|---|---|
| Trastuzumab | 43 g/L |
| Trastuzufab | 48 g/L |
| Nucala ® | 34 g/L |
| Benlysta ® | 37 g/L |

### Example 14. NaOH Stability of the Affinity Resin Column

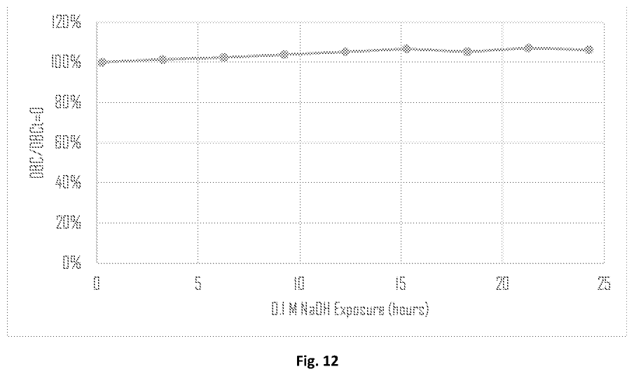

This example demonstrates the NaOH stability of the affinity resin column in the previous example. Repeated DBC cycles were performed at the indicated time points after exposure of the resin to 0.1 M NaOH containing 1 M NaCl and the DBC at 10% breakthrough determined. The data shown in FIG. 12 demonstrates that the resin shows no loss in binding capacity over 24-hour exposure to 0.1 M NaOH containing 1 M NaCl.

### Example 15. Purification of an Antibody

This example demonstrates the purification of an antibody from a crude feedstream and that the resin can be reused many times. The affinity resin of Example 13 was packed into a 0.3×10 cm column and challenged with a host cell clarified feed (HCCF) containing Trastuzumab at a titer of 0.8 g/L. The column was operated as shown in the following table.

| | | Residence | |
|---|---|---|---|
| | | Time | |
| Step | Buffer | (min) | CV |
| Equilibration | 20 mM Tris, pH 7.4 | 1 | 7 |
| Load Sample | HCCF (0.8 mg/mL) | 4 | 44 |
| Chase | 20 mM Tris, pH 7.4 | 4 | 5 |
| Wash | 50 mM HEPES, 1M CaCl2 pH 7.5 | 1 | 7 |
| Elution | 100 mM glycine, 150 mM NaCl | 2 | 7 |
| | pH 3 | | |
| Strip | 0.1M NaOH, 1M NaCl | 4 | 7.6 |
| Re-Equilibration | 20 mM Tris, pH 7.4 | 2 | 14.3 |

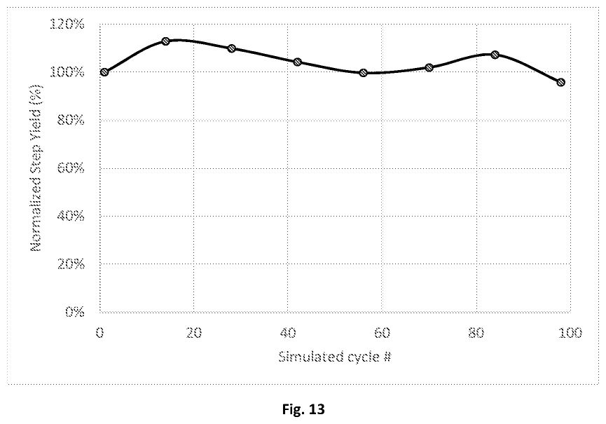

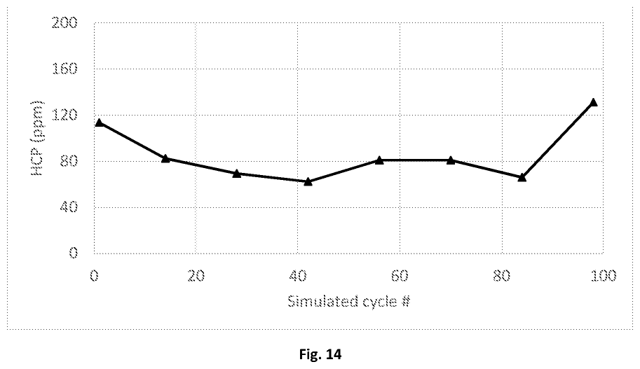

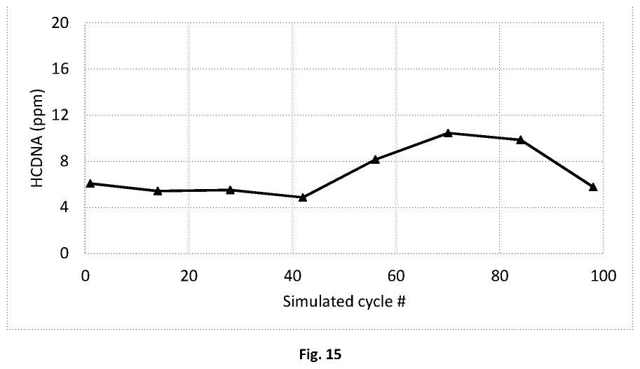

During the “Strip” step the flow was stopped so that the total exposure of the resin to 0.1 M NaOH containing 1 M NaCl was 3.5 hours per cycle and the column was operated for 8 cycles. The yield obtained at each cycle is shown in FIG. 13. The residual HCP and DNA impurity levels obtained at each cycle are shown in FIGS. 14 and 15, respectively. Collectively, the data show high purity and the ability to cycle the column without loss in performance.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims and list of embodiments disclosed herein. Although specific terms are employed herein, they are used in a generic and descriptive sense only and not for purposes of limitation.

Any methods disclosed herein need not be performed in the order recited. The methods disclosed herein include certain actions taken by a practitioner; however, they can also include any third-party instruction of those actions, either expressly or by implication.

TABLE 8
SEQUENCES

| SEQ | |
|---|---|
| ID | AA Sequence |
| 1 | X¹RWX²X³ |
| 2 | X⁴AWX⁵X⁶LGX⁷ |
| 3 | X⁸AWX⁹X¹⁰LGX¹¹ |
| 4 | X¹²ARX¹³X¹⁴LEX¹⁵ |
| 5 | X¹RWX²X³LGAAYAARGDYDRAIEYYQRALELDPNNAX⁴AWX⁵X⁶LGX⁷AYAARGDYDRAIE |
| | YYQRALELDPNNAX⁸AWX⁹X¹⁰LGX¹¹AYAARGDYDRAIEYYQRALELDPNNEX¹²ARX¹³x¹⁴ |
| | LEX¹⁵ |
| 6 | MAQAAAGDEADRWADLGAAYAARGDYDRAIEYYERALELDPNNAWAWWDLGVAYAARGDY |
| | DRAIEYYERALELDPDDATAWSRLGHAYAARGDYDRAIEYYERALELDPEDETARYLLEH |
| | ARRARQGQAGQGGGSGLNDIFEAQKIEWHECHHHHHH |
| 7 | MAQAAAGDEADRWADLGAAYAARGDYDRAIEYYQRALELDPNNAWAWWDLGVAYAARGDY |
| | DRAIEYYQRALELDPNNATAWNRLGHAYAARGDYDRAIEYYERALELDPEDETARYLLEH |
| | ARRARQGQAGQGGGSGLNDIFEAQKIEWHECHHHHHH |
| 8 | MAQAAAGDEAGRWEGQGAAYAARGDYDRAIEYYQRALELDPNNAWAWWELGVAYAARGDY |
| | DRAIEYYQRALELDPNNATAWNRLGHAYAARGDYDRAIEYYQRALELDPNNEVARSQLEH |
| | ARRARRGQAGQGGGSGLNDIFEAQKIEWHECHHHHHH |
| 9 | MAQAAAGDEAGRWEGQGAAYAARGDYDRAIEYYERALELDPNNAWAWWDLGVAYAARGDY |
| | DRAIEYYERALELDPDDATAWSRLGHAYAARGDYDRAIEYYERALELDPEDETARYLLEH |
| | ARRARQGQAGQGGGSGLNDIFEAQKIEWHECHHHHHH |
| 10 | MAQAAAGDEAGRWENQGAAYAARGDYDRAIEYYERALELDPNNAWAWWELGVAYAARGDY |
| | DRAIEYYERALELDPDDAHAWRRLGFAYAARGDYDRAIEYYERALELDPEDEVAREVLEE |
| | ARRARQGQAGQGGGSGLNDIFEAQKIEWHECHHHHHH |
| 11 | MAQAAAGDEAARWEWQGAAYAARADYDRAIEYYERALELDPNNAWAWWELGVAYAARGEL |
| | DRAIEYYERALELDPDDAHAWFRLGWAYAARGDYDRAIEYYQRALELDPEDELAREELEE |
| | ARRARQGQAGQGGGSGLNDIFEAQKIEWHECHHHHHH |
| 12 | MAQAAAGDEAQRWENQGAAYAARGDYDRAIEYYERALELDPNNAWAWWELGVAYAARGDY |
| | DRAIEYYERALELDPDDATAWSRLGHAYAARGDYDRAIEYYERALELDPEDETARYLLEH |
| | ARRARQGQAGQGGGSGLNDIFEAQKIEWHECHHHHHH |
| 13 | MAQAAAGDEAARWEWQGAAYAARGDYDRAIEYYERALELDPNNAWAWWELGVAYAARGEL |
| | DRAIEYYERALELDPDDAHAWFRLGWAYAARGDYDRAIEYYERALELDPEDELAREELEE |
| | ARRARQGQAGQGGGSGLNDIFEAQKIEWHECHHHHHH |
| 14 | MAQAAAGDEAQRWENQGAAYAARGDYDRAIEYYERALELDPNNAWAWWELGVAYAARGDY |
| | DRAIEYYERALELDPDDAAAWRRLGHAYAARGDYDRAIEYYERALELDPEDETARYLLEH |
| | ARRARQGQAGQGGGSGLNDIFEAQKIEWHECHHHHHH |
| 15 | MAQAAAGDEAQRWENQGAAYAARGDYDRAIEYYERALELDPNNAWAWWDLGVAYAARGDY |
| | DRAIEYYERALELDPDDAAAWRRLGHAYAARGDYDRAIEYYERALELDPEDELARRILED |
| | ARRARQGQAGQGGGSGLNDIFEAQKIEWHECHHHHHH |
| 16 | MAQAAAGDEAGRWEGQGAAYAARGDYDRAIEYYERALELDPNNAWAWWELGVAYAARGDY |
| | DRAIEYYERALELDPDDAAAWRRLGHAYAARGDYDRAIEYYERALELDPEDELARRILED |
| | ARRARQGQAGQGGGSGLNDIFEAQKIEWHECHHHHHH |
| 17 | MAQAAAGDEAGRWEGQGAAYAARGDYDRAIEYYERALELDPNNAWAWWELGVAYAARGEL |
| | DRAIEYYERALELDPDDAHAWFRLGWAYAARGDYDRAIEYYQRALELDPNNEVARSQLEH |
| | ARRARRGQAGQGGGSGLNDIFEAQKIEWHECHHHHHH |
| 18 | MAQAAAGDEAQRWENQGAAYAARGDYDRAIEYYERALELDPNNAWAWWELGVAYAARGEL |
| | DRAIEYYERALELDPDDAHAWFRLGWAYAARGDYDRAIEYYERALELDPEDELAREELEE |
| | ARRARRGQAGQGGGSGLNDIFEAQKIEWHECHHHHHH |
| 19 | MAQAAAGDEAGRWEGQGAAYAARGDYDRAIEYYERALELDPNNAWAWWDLGVAYAARGDY |
| | DRAIEYYERALELDPDDATAWSRLGHAYAARGDYDRAIEYYERALELDPEDELARQQLEW |
| | ARRARQGQAGQGGGSGLNDIFEAQKIEWHECHHHHHH |
| 20 | MAQAAAGDEAQRWETLGAAYAARGDYDRAIEYYQRALELDPNNAWAWWELGVAYAARGDY |
| | DRAIEYYERALELDPDDATAWSRLGHAYAARGDYDRAIEYYQRALELDPNNEVARSQLEH |
| | ARRARRGQAGQGGGSGLNDIFEAQKIEWHECHHHHHH |
| 21 | MAQAAAGDEALRWWDQGAAYAARGDYDRAIEYYERALELDPNNAWAWWDLGVAYAARGDY |
| | DRAIEYYERALELDPDDAHAWFRLGWAYAARGDYDRAIEYYERALELDPEDELAREELEE |
| | ARRARQGQAGQGGGSGLNDIFEAQKIEWHECHHHHHH |
| 22 | MAQAAAGDEAGRWEGQGAAYAARGDYDRAIEYYERALELDPNNAWAWWELGTAYAARGDY |
| | DRAIEYYERALELDPDDAIAWGRLGHAYAARGDYDRAIEYYERALELDPEDELARQQLEW |
| | ARRARQGQAGQGGGSGLNDIFEAQKIEWHECHHHHHH |
| 23 | MAQAAAGDEAGRWEGQGAAYAARGDYDRAIEYYERALELDPNNAWAWWDLGVAYAARGDY |
| | DRAIEYYERALELDPDDATAWNRLGHAYAARGDYDRAIEYYQRALELDPNNEVARSQLEH |
| | ARRARRGQAGQGGGSGLNDIFEAQKIEWHECHHHHHH |
| 24 | MAQAAAGDEAQRWENQGAAYAARGDYDRAIEYYERALELDPNNAWAWWDLGVAYAARGDY |
| | DRAIEYYERALELDPDDATAWSRLGHAYAARGDYDRAIEYYERALELDPEDETARYLLEH |
| | ARRARQGQAGQGGGSGLNDIFEAQKIEWHECHHHHHH |
| 25 | MAQAAAGDEAQRWETLGAAYAARGDYDRAIEYYQRALELDPNNAWAWWELGVAYAARGDY |
| | DRAIEYYQRALELDPDDAAAWRRLGHAYAARGDYDRAIEYYERALELDPEDELARRILED |
| | ARRARQGQAGQGGGSGLNDIFEAQKIEWHECHHHHHH |
| 26 | MAQAAAGDEAGRWEGQGAAYAARGDYDRAIEYYERALELDPNNAWAWWDLGVAYAARGDY |
| | DRAIEYYERALELDPDDAAAWNRLGHAYAARGDYDRAIEYYQRALELDPNNEVARSQLEH |
| | ARRARRGQAGQGGGSGLNDIFEAQKIEWHECHHHHHH |
| 27 | MAQAAAGDEAQRWENQGAAYAARGDYDRAIEYYERALELDPNNAWAWWELGVAYAARGDY |
| | DRAIEYYERALELDPDDAAAWRRLGHAYAARGDYDRAIEYYERALELDPEDELAREELEE |
| | ARRARQGQAGQGGGSGLNDIFEAQKIEWHECHHHHHH |
| 28 | MAQAAAGDEAQRWENQGAAYAARGDYDRAIEYYERALELDPNNAWAWWELGVAYAARGDY |
| | DRAIEYYERALELDPDDATAWNRLGHAYAARGDYDRAIEYYERALELDPEDETARYLLEH |
| | ARRARQGQAGQGGGSGLNDIFEAQKIEWHECHHHHHH |
| 29 | MAQAAAGDEAGRWEGQGAAYAARGDYDRAIEYYERALELDPNNAWAWWDLGVAYAARGDY |
| | DRAIEYYERALELDPDDAAAWRRLGHAYAARGDYDRAIEYYERALELDPEDELARRILED |
| | ARRARQGQAGQGGGSGLNDIFEAQKIEWHECHHHHHH |
| 30 | MAQAAAGDEAARWEWQGAAYAARGDYDRAIEYYERALELDPNNAWAWWELGNAYAARGDY |
| | DRAIEYYERALELDPDDATAWSRLGHAYAARGDYDRAIEYYERALELDPEDETARYLLEH |
| | ARRARRGQAGQGGGSGLNDIFEAQKIEWHECHHHHHH |
| 31 | MAQAAAGDEAARWEWQGAAYAARGDYDRAIEYYERALELDPNNAWAWWELGVAYAARGEL |
| | DRAIEYYERALELDPDDAHAWFRLGWAYAARGDYDRAIEYYQRALELDPEDELARRILED |
| | ARRARQGQAGQGGGSGLNDIFEAQKIEWHECHHHHHH |
| 32 | MAQAAAGDEAVRWEIQGAAYAARGDYDRAIEYYERALELDPNNAWAWWELGVAYAARGDY |
| | DRAIEYYERALELDPDDAHAWFRLGWAYAARGDYDRAIEYYERALELDPEDELARRILED |
| | ARRARQGQAGQGGGSGLNDIFEAQKIEWHECHHHHHH |
| 33 | MAQAAAGDEAQRWETLGAAYAARGDYDRAIEYYQRALELDPNNAWAWWELGVAYAARGDY |
| | DRAIEYYQRALELDPNNATAWNRLGHAYAARGDYDRAIEYYQRALELDPNNEVARSQLEH |
| | ARRARRGQAGQGGGSGLNDIFEAQKIEWHECHHHHHH |
| 34 | MAQAAAGDEAGRWEGQGAAYAARGDYDRAIEYYERALELDPNNAWAWWDLGVAYAARGDY |
| | DRAIEYYERALELDPDDAAAWRRLGHAYAARGDYDRAIEYYERALELDPEDETARYLLEH |
| | ARRARQGQAGQGGGSGLNDIFEAQKIEWHECHHHHHH |
| 35 | MAQAAAGDEAARWEWQGAAYAARGDYDRAIEYYERALELDPNNAWAWWELGVAYAARGEL |
| | DRAIEYYERALELDPDDAHAWFRLGWAYAARGDYDRAIEYYERALELDPEDELARRILED |
| | ARRARQGQAGQGGGSGLNDIFEAQKIEWHECHHHHHH |
| 36 | MAQAAAGDEAQRWENQGAAYAARGDYDRAIEYYERALELDPNNAWAWWELGVAYAARGDY |
| | DRAIEYYERALELDPDDAAAWRRLGHAYAARGDYDRAIEYYERALELDPEDELARRILED |
| | ARRARQGQAGQGGGSGLNDIFEAQKIEWHECHHHHHH |
| 37 | MAQAAAGDEAQRWENQGAAYAARGDYDRAIEYYERALELDPNNAWAWWELGVAYAARGEL |
| | DRAIEYYERALELDPDDAHAWFRLGWAYAARGDYDRAIEYYERALELDPEDELARRILED |
| | ARRARQGQAGQGGGSGLNDIFEAQKIEWHECHHHHHH |
| 38 | MAQAAAGDEAQRWETLGAAYAARGDYDRAIEYYQRALELDPNNAWAWWELGVAYAARGDY |
| | DRAIEYYERALELDPDDAAAWRRLGHAYAARGDYDRAIEYYERALELDPEDELARRILED |
| | ARRARQGQAGQGGGSGLNDIFEAQKIEWHECHHHHHH |
| 39 | MAQAAAGDEAQRWEHLGAAYAARGDYDRAIEYYQRALELDPNNAWAWWELGVAYAARGDY |
| | DRAIEYYQRALELDPNNAVAWNRLGHAYAARGDYDRAIEYYQRALELDPNNEFARQSLEE |
| | ARRARRGQAGQGGGSGLNDIFEAQKIEWHECHHHHHH |
| 40 | MAQAAAGDEANRWYVLGAAYAARGDYDRAIEYYQRALELDPNNAYAWDELGSAYAARGDY |
| | DRAIEYYQRALELDPNNAVAWSRLGQAYAARGDYDRAIEYYQRALELDPNNETARHLLEW |
| | ARRARRGQAGQGGGSGLNDIFEAQKIEWHECHHHHHH |
| 41 | MAQAAAGDEAERWYDLGAAYAARGDYDRAIEYYQRALELDPNNAWAWWELGVAYAARGDY |
| | DRAIEYYQRALELDPNNAVAWARLGIAYAARGDYDRAIEYYQRALELDPNNEVARDALEY |
| | ARRARRGQAGQGGGSGLNDIFEAQKIEWHECHHHHHH |
| 42 | MAQAAAGDEAERWESQGAAYAARGDYDRAIEYYERALELDPNNAWAWWELGTAYAARGDY |
| | DRAIEYYERALELDPDDARAWSRLGHAYAARGDYDRAIEYYERALELDPEDETARANLED |
| | ARRARRGQAGQGGGSGLNDIFEAQKIEWHECHHHHHH |
| 43 | MAQAAAGDEAQRWEEQGAAYAARGDYDRAIEYYERALELDPNNAWAWWELGTAYAARGDY |
| | DRAIEYYERALELDPDDARAWSRLGHAYAARGDYDRAIEYYERALELDPEDETARFLLEQ |
| | ARRARRGQAGQGGGSGLNDIFEAQKIEWHECHHHHHH |
| 44 | MGGGGSAAAGDEAIRWNTLGYAYGWRGDYDRAIEYYQRALELDPNNATAWAALGYAYQER |
| | GDYDRAIEYYQRALELDPNNAHAWQYLGFAYWNRGDYDRAIEYYQRALELDPNNESARSY |
| | LEDARRTRRGQAGQGGGSGLNDIFEAQKIEWHECHHHHHH |
| 45 | MGGGGSAAAGDEADRWYRLGSAYWDRGDYDRAIEYYQRALELDPNNAGAWYWLGSAYWHR |
| | GDYDRAIEYYQRALELDPNNAYAWSELGHAYIDRGDYDRAIEYYQRALELDPNNEFARGQ |
| | LEGARRLRRGQAGQGGGSGLNDIFEAQKIEWHECHHHHHH |
| 46 | MGGGGSAAAGDEADRWSTQGAAYAARGDYDRAIEYYQRALELDPNNAWAWWDLGVAYAAR |
| | GDYDRAIEYYQRALELDPNNAVAWARLGIAYAARGDYDRAIEYYQRALELDPNNEVARDA |
| | LEYARRARRGQAGQGGGSGLNDIFEAQKIEWHECHHHHHH |
| 47 | MGGGGSAAAGDEAGRWEGQGAAYAARGDYDRAIEYYERALELDPNNAWAWWDLGVAYAAR |
| | GDYDRAIEYYQRALELDPNDATAWNRLGHAYAARGDYDRAIEYYQRALELDPNNEVARQL |
| | LEYARRARRGQAGQGGGSGLNDIFEAQKIEWHECHHHHHH |
| 48 | MGGGGSAAAGDEAVRWEELGAAYAARGDYDRAIEYYQRALKLDPNNAWAWWELGVAYAAR |
| | GDYDRAIEYYQRALELDPNNAVAWARLGWAYAARGDYDRAIEYYQRALELDPNNESARTG |
| | LKLARRARRGQAGQGGGSGLNDIFEAQKIEWHECHHHHHH |
| 49 | MGGGGSAAAGDEAFRWWDQGAAYTARGDYDRAIEYYQRALELDPNNAWAWWELGVAYAAR |
| | GDYDRAIEYYQRALELDPNNAVAWNRLGHAYAARGDYDRAIEYYQRALELDPNNEVARDQ |
| | LERARRARRGQAGQGGGSGLNDIFEAQKIEWHECHHHHHH |
| 50 | MGGGGSAAAGDEAARWEWQGAAYAARGDYDHAIEYYERALELDPNNAWAWWELGVAYAAR |
| | GELDRAIEYYERALELDPDDAHAWFRLGWAYAARGDYDRAIEYYERALELDPEDELAREE |
| | LEEARRARQGQAGQGGGSGLNDIFEAQKIEWHECHHHHHH |
| 51 | MGGGGSAAAGDEADRWADLGAAYAARGDYDRAIEYYQRALELDPNNAWAWWDLGVAYAAR |
| | GDYDRAIEYYQRALELDPNNAVAWRRLGHAYAARGDYDRAIEYYQRALELDPNNELAREQ |
| | LERARRARRGQAGQGGGSGLNDIFEAQKIEWHECHHHHHH |
| 52 | MGGGGSAAAGDEADRWADLGAAYAARGDYDRAIEYYQRALELDPNNAWAWWDLGVAYAAR |
| | GDYDRAIEYYQRALELDPNNAVAWRRLGHAYAARGDYDRATEYYQRALELDPNNELAREQ |
| | LEHARRARRGQAGQGGGSGLNDIFEAQKIEWHECHHHHHH |
| 53 | MGGGGSAAAGDEAERWYDLGAAYAARGDYDRAIEYYQRALELDPNNAWAWWELGVAYAAR |
| | GDYDRAIEYYQRALELDPNNAVAWSRLGQAYAARGDYDRAIEYYQRALELDPNNETARHL |
| | LEWARRARRGQAGQGGGSGLNDIFEAQKIEWHECHHHHHH |
| 54 | MGGGGSAAAGDEAARWEVQGAAYAARGDYDRAIEYYERALELDPNNAWAWEHLGWAYAAR |
| | GDYDRAIEYYERALELDPDDAHAWFRLGFAYAARGDYDRAIEYYERALELDPEDELARAE |
| | LESARRARQGQAGQGGGSGLNDIFEAQKIEWHECHHHHHH |
| 55 | MGGGGSAAAGDEANRWYYLGAAYAARGDYDRAIEYYQRALELDPNNAFAWDELGNAYAAR |
| | GDYDRAIEYYQRALELDPNNAVAWNRLGHAYAARGDYDRAIEYYQRALELDPNNEVARTL |
| | LEQARRARRGQAGQGGGSGLNDIFEAQKIEWHECHHHHHH |
| 56 | MGGGGSAAAGDEAWRWYSQGAAYAARGDYDRAIEYYERALELDPNNAWAWWELGTAYAAR |
| | GDYDRAIEYYERALELDPDDAIAWGRLGHAYAARGDYDRAIEYYERALELDPEDELARQQ |
| | LEWARRARQGQAGQGGGSGLNDIFEAQKIEWHECHHHHHH |
| 57 | MGGGGSAAAGDEADRWWALGAAYAARGDYDRAIEYYQRALELDPNNAWAWWDLGVAYAAR |
| | GDYDRAVEYYQRALELDPNNAVAWRRLGHAYAARGDYDRAIEYYQRALELDPNNELAREQ |
| | LERARRARHGQAGQGGGSGLNDIFEAQKIEWHECHHHHHH |
| 58 | MGGGGSAAAGDEANRWYYLGAAYAARGDYDRAIEYYQRALELDPNNAFAWDELGNAYAAR |
| | EDYDRAIEYYQRALELDPNNAVAWNRLGHAYAARGDYDRAIEYYQRALELDPNNEVARTL |
| | LEQARRARRGQAGQGGGSGLNDIFEAQKIEWHECHHHHHH |
| 59 | MGGGGSAAAGDEAERWYDLGAAYAARGDYDRAIEYYQRALELDPNNAWAWWELGVAYAAR |
| | GDYDRAIEYYQRALELDPNNAVAWARLGIAYAARGDYDRAIEYYQRALELDPNNEVARTL |
| | LEQARRARRGQAGQGGGSGLNDIFEAQKIEWHECHHHHHH |
| 60 | MGGGGSAAAGDEAGRWEGQGAAYAARGDYDRAIEYYERALELDPNNAWAWWDLGVAYAAR |
| | GDYDRAIEYYERALELDPDDATAWSRLGHAYAARGDYDRAIEYYERALQLDPEDETARYL |
| | LEHARRARQGQAGQGGGSGLNDIFEAQKIEWHECHHHHHH |
| 61 | MGGGGSAAAGDEADRWYELGAGYAARGDYDRAIEYYQRALELDPNNAWAWWELGVAYAAR |
| | GDYDRAIEYYQRALELDPNNATAWQRLGHAYAARGDYDRAIEYYQRALELDPNNEAARYQ |
| | LEFARRARRGQAGQGGGSGLNDIFEAQKIEWHECHHHHHH |
| 62 | MGGGGSAAAGDEASRWEIQGAAYAARGDYDRAIEYYERALELDPNNAWAWWDLGVAYAAR |
| | GDYDRAIEYYQRALELDPNNAVAWRRLGHAYAARGDYDRAIEYYQRALELDPNNELAREQ |
| | LERARRARRGQAGQGGGSGLNDIFEAQKIEWHECHHHHHH |
| 63 | MGGGGSAAAGDEADRWADLGAAYAARGDYDRAIEYYQRALELDPNNAWAWWDLGVAYAAR |
| | GDYDRAIEYYQRALELDPNNAVAWRRLGHAYAARGDYDRAIEYYQRALELDPNNELAREQ |
| | LERARRARHGQAGQGGGSGLNDIFEAQKIEWHECHHHHHH |
| 64 | MGGGGSAAAGDEADRWAALGAAYAARGDYDRAIEYYQRALELDPNNAWAWWDLGVAYAAR |
| | GDYDRAIEYYERALELDPDDAHAWFRLGWAYAARGDYDRAIEYYERALELDPEDETARDV |
| | LEGARRARQGQAGQGGGSGLNDIFEAQKIEWHECHHHHHH |
| 65 | MGGGGSAAAGDEAERWYDLGAAYAARGDYDRAIEYYQRALELDPNNAWAWWELGVAYAAR |
| | GDYDRAIEYYQRALELDPNNAVAWRRLGIAYAARGDYDRAIEYYQRALELDPNNEVARDA |
| | LEYARRARRGQAGQGGGSGLNDIFEAQKIEWHECHHHHHH |
| 66 | MGGGGSAAAGDEAGRWYDLGAAYAARGDYDRAIEYYQRALELDPNNAWAWWELGVAYAAR |
| | GDYDRAIEYYQRALELDPNNAVAWARLGIAYAARGDYDRAIEYYQRALELDPNNEVARDA |
| | LEYARRARRGQAGQGGGSGLNDIFEAQKIEWHECHHHHHH |
| 67 | MGGGGSAAAGDEADRWADLGAAYAARGDYDRAIEYYQRALELDPNNAWAWWDLGVAYAAR |
| | GDYDRAIEYYQRALELDPNNAVAWRRLGHAYAARGDYDRAIEYYQRALELDPNNELAREQ |
| | LGRARRARRGQAGQGGGSGLNDIFEAQKIEWHECHHHHHH |
| 68 | MGGGGSAAAGDEGGRWEGQGAAYAARGDYDRAIEYYERALELDPNNAWAWWDLGVAYAAR |
| | GDYDRAIEYYERALELDPDDATAWSRLGHAYAARGDYDRAIEYYERALELDPEDETARYL |
| | LEHARRARQGQAGQGGGSGLNDIFEAQKIEWHECHHHHHH |
| 69 | MGGGGSAAAGDEAERWDYLGAAYAARGDYDRAIEYYQRALELDPNNAWAWWDLGLAYAAR |
| | GDYDRAIEYYQRALELDPNNAVAWFRLGWAYAARGDYDRAIEYYQRALELDPNNELARSE |
| | LENARRARRGQAGQGGGSGLNDIFEAQKIEWHECHHHHHH |
| 70 | MGGGGSAAAGDEAQRWENQGAAYAARGDYDRAIECYESALELDPNNAWAWWELGVAYAAR |
| | GDYDRAIEYYERALELDPDDAIAWSRLGHAYAARGDYDRAIEYYERALELDPEDETARYL |
| | LEHARRARQGQAGQGGGSGLNDIFEAQKIEWHECHHHHHH |
| 71 | MGGGGSAAAGDEAQHWENQGAAYAARGDYDRAIEYYERALELDPNNAWAWWELGVAYAAR |
| | GDYDRAIEYYERALELDPDDATVWSRLGHAYAARGDYDRAIEYYERALELDPEDETARYL |
| | LEHARRARQGQAGQGGGSGLNDIFEAQKIEWHECHHHHHH |
| 72 | MGGGGSAAAGDEAQCWENQGAAYAARGDYDRAIEYYERALELDPNNAWAWWELGVAYAAR |
| | GDYDRAIEYYERALELDPDDATAWSRLGHAYAARGDYDRAIEYYERALELDPEDETARYL |
| | LEHARRARQGQAGQGGGSGLNDIFEAQKIEWHECHHHHHH |
| 73 | MGGGGSAAAGDEAQHWENQGAAYAARGDYDRAIEYYGRALELDPNNAWAWWELGVAYAAR |
| | GDYDRAIEYYERALELDPDDATTWSRLGHAYAARGDYDRAIEYYERALELDPEDETARYL |
| | LEHARRARQGQAGQGGGSGLNDIFEAQKIEWHECHHHHHH |
| 74 | MGGGGSAAAGDEAQCWENQGAAYAARGDYDRAIEYYERALELDPNNAWAWWELGVAYAAR |
| | GDYDRAIEYYERALELDPDDATTWSRLGHAYAARGDYDRAIEYYVRALELDPEDETARYL |
| | LEHARRARQGQAGQGGGSGLNDIFEAQKIEWHECHHHHHH |
| 75 | MGGGGSAAAGDEAQRWENQGAAYAARGDYDRAIEYYGRALELDPNNAWAWWELGVAYAAR |
| | GDYDRAIEYYERALELDPDDATAWSRLGHAYAARGDYDRAIEYYERALELDPENETARYL |
| | LEHARRARQGQAGQGGGSGLNDIFEAQKIEWHECHHHHHH |
| 76 | MGGGGSAAAGDEAQRWENQGAAYAARGDYDRAIEYYERALELEPNNAWAWWELGVAYAAR |
| | GDYDRAIEYYERALELDPDDATAWSRLGHAYAARGDYDRAIGYYERALELDPENETARYL |
| | LEHARRARQGQAGQGGGSGLNDIFEAQKIEWHECHHHHHH |
| 77 | MGGGGSAAAGDEAQRWENQGAAYAARGDYDRAIEYYERALELDPNNAWAWWELGVAYAAR |
| | GDFDRAIEYYERALELDPDDATVWSRLGHAYAARGDYDRAIEYYERALELDPVDETARYL |
| | LEHARRARQGQAGQGGGSGLNDIFEAQKIEWHECHHHHHH |
| 78 | MGGGGSAAAGDEAVRWEIQGAAYAARGDYDRAIEYYERALELDPNNAWAWWELGVAYAAR |
| | GDYDRAIEYYERALELDPDDAHVWFRLGWAYAVRGDYDRAIEYYERALELDPEDELARRI |
| | LEDARRARQGQAGQGGGSGLNDIFEAQKIEWHECHHHHHH |
| 79 | MGGGGSAAAGDEAVRWEIQGAAYAARGDYNRAIEYYERALELDPNNAWAWWELGVAYAAR |
| | GDYDRAIEYYERALNLDPDDAHTWFRLGWAYAARGDYARAIEYYERALELDPEDELVRRI |
| | LEDARRARQGQAGQGGGSGLNDIFEAQKIEWHECHHHHHH |
| 80 | MGGGGSAAAGDEAVRWEIQGAAYAARGDYDLAIEYYERTLELDPDNAWAWWELGVAYAAR |
| | GDYDRAIEYYERALELDPDDAHVWFRLGWAYATRGDYDRAIEYYERALELDPEDELARRI |
| | LEDARRARQGQAGQGGGSGLNDIFEAQKIEWHECHHHHHH |
| 81 | MGGGGSAAAGDEAARWEIQGAAYAARGDYDRAIEYYERALELDPNNAWAWWELGVAYAAR |
| | GDYDRAIEYYERALELDPDDAHTWFRLGWAYAARGDYDRAIAYYERALELDPEDELTRRI |
| | LGDARRVRQGQAGQGGGSGLNDIFEAQKIEWHECHHHHHH |
| 82 | MGGGGSAAAGDEAVRWEIQGAAYAARGDYDRAIEYYERALELDPNNAWAWWELGVAYAAR |
| | GDYDRAIEYYERALELDPDDARVWFRLGWAYAARGDYDRAIEYYERALELDPEDELARRI |
| | LEDTRRARQGQAGQGGGSGLNDIFEAQKIEWHECHHHHHH |
| 83 | MGGGGSAAAGDEVVHWEIQGAAYAARGDYDRAIEYYERALELDPNNAWAWWELGVAYAAR |
| | GDYDRAIEYYERALELDPDDAHVWFRLGWAYAARGDYDRAIEYYERALELDPEDELARRI |
| | LEDARRARQGQAGQGGGSGLNDIFEAQKIEWHECHHHHHH |
| 84 | MGGGGSAAAGDEAVRWEIQGAAYAARGDYDRAIEYYERALELDPNNAWAWWELGVAYAAR |
| | GDYDRAIEYYERALELDPDDAHVWFRLGWAYATRGDYDRAIEYYERALELDPEDELARRI |
| | LEDARRARQGQAGQGGGSGLNDIFEAQKIEWHECHHHHHH |
| 85 | MGGGGSAAAGDEALRWEIQGAAYAARGDYDRAIEYYERALELDPNNAWAWWELGVAYAAR |
| | GDYDRAIEYYERALELDPDDAHVWFRLGWAYAARGDHDRAIEYYERALELDPEDELARRI |
| | LEDARRARQGQAGQGGGSGLNDIFEAQKIEWHECHHHHHH |
| 86 | MGGGGSAAAGDEAERWYDLGAAYAARGDYDRAIEYYQRALELDPNDAWAWWELGVAYAAR |
| | GDYDRAIEYYQRALELDLNNAVAWARLGIAYAARGDYDRAIEYYQRALELDPNNEVARDA |
| | LEYARRARRGQAGQGGGSGLNDIFEAQKIEWHECHHHHHH |
| 87 | MGGGGSAAAGDEAERWYDLGAAYAARGDYDRAFEYYQRALELDPNNAWAWWELGVANAAR |
| | GDYDRAIENYQRALELDPNNAVAWARLGVAYAARGDYDRAIEYYQRALELDPNQVIARGA |
| | LEYARRARRGQAGQGGGSGLNDIFEAQKIEWHECHHHHHH |
| 88 | MGGGGSAAAGDEAERWYDLGAAYAARGDYDRAIEYYQRVLELDPNNAWAWWELGVGYAAR |
| | GDYDRAIEYYQRALELDPNNTVAWARLGIAYAARGDYDRAIEYYQRALELDPNNEVARDA |
| | LEYARRARRGQAGQGGGSGLNDIFEAQKIEWHECHHHHHH |
| 89 | MGGGGSAAAGDEAERWYDLGAAYAARGDEDRAIEYYRRTLELDPNDAWAWWELGVAYAAR |
| | GDYDRAIEYYQRALELDPNNAVAWARLGIAYATRGDYDRAIEYYQRALELDPNNEVARDA |
| | LEYARRVRRGQAGQGGGSGLNDIFEAQKIEWHECHHHHHH |
| 90 | MGGGGSAAAGDEAERWYDLGAAYAARGDYDRATEFYQRALELDPNDAWAWWELGIAYAAR |
| | GDYDRAIEYYQRALELDPNNAVAWARLGIAYAARGDYDRAIEYYQRALELDPNNEVARDA |
| | LEYARRARRGQAGQGGGSGLNDIFEAQKIEWHECHHHHHH |
| 91 | MGGGGSAAAGDEAERWYDLGAAYAARGDYDRAIEYYQRALELDPNDAWAWWELGVAYAAR |
| | GDYDRAIEYYQRALELDPNNAVAWARLGIAYAARGDYDRAIEYYQRALELDPNNEVARDA |
| | LEYARRARRGQAGQGGGSGLNDIFEAQKIEWHECHHHHHH |
| 92 | MGGGGSAAAGDEAERWYDLGAAYAARGDYDRAIEYYQRALEHDPNDAWAWWELGVAYAAR |
| | GDYDRAIEYYQRALELDTNNAVAWARLGIAYAARGDYDRAIEYYQRALELDPNNEVARDA |
| | LEYARRARRGQAGQGGGSGLNDIFEAQKIEWHECHHHHHH |
| 93 | MGGGGSAAAGDEAAHWEFQGAAYAARGDYDRAIEYYERALELDPNNAWAWEHLGWAYAAR |
| | GDYDRAIEYYERALELDPDDAHAWFRLGFAYAARGDYDRAIKYYERALELDPEDELARAE |
| | LESARRARQGQAGQGGGSGLNDIFEAQKIEWHECHHHHHH |
| 94 | MGGGGSAAAGDEAARWEVQGAAYAARGDYDRAIEYYERVLELDPNDAWAWEHLGWAYAAR |
| | GDYDRAIEYYERALEVDPDDAHAWFRLGFAYAARGDYDRAMEYYERALELDPEDELARAE |
| | LESARRARQGQAGQGGGSGLNDIFEAQKIEWHECHHHHHH |
| 95 | MGGGGSAAAGDEAARWEVQGAAYAARGDYDRAIEYYERALELDPNNAWAWEHLGWAYAAR |
| | GDYDRAIEYYERALELDPDDAHAWFRLGFAYAARGDYDRAIEYYERALELDPNNELAREQ |
| | LEHARRARRGQAGQGGGSGLNDIFEAQKIEWHECHHHHHH |
| 96 | MGGGGSAAAGDEAAHWEVQGAAYAARGDYDRAIEYYERALELDPNNAWAWEHLGWAYAAR |
| | GDFDRAIEYYERALELDPDDAHAWFRLGFAYAARGDYDRAIEYYERALELDPEDELARAE |
| | LESARRARQGQAGQGGGSGLNDIFEAQKIEWHECHHHHHH |
| 97 | MGGGGSAAAGDEAARWEVLGAAYAARGDYDRAIEYYERALELDPNNAWAWEHLGWAYAAR |
| | GDYDRAIEYYERALGLDPDDAHAWFRLGFAYAARGDYDRAIEYYERALELDPENELARAE |
| | LESARRARQGQAGQGGGSGLNDIFEAQKIEWHECHHHHHH |
| 98 | MGGGGSAAAGDEAARWEVQGAAYAARGDYDRAIEYYERALELDPNNAWAWEHLGWAYAAR |
| | GDYDRAIEYYERALELDPDDAHSWFRLGFAYAVRGDYDRAIEYYERALELDPEDELARAE |
| | LESARRARQGQAGQGGGSGLNDIFEAQKIEWHECHHHHHH |
| 99 | MGGGGSAAAGDEVARWEVQGAAYAARGGYDRAIEYYERALELDPNNAWAWEHLGWAYAAR |
| | GDYDRAIEYYERALELDPDDAHEWFRLGFAYAARGDYDRAIEYYERALELDPEDELARAE |
| | LESARRARQGQAGQGGGSGLNDIFEAQKIEWHECHHHHHH |
| 100 | MGGGGSAAAGDEAARWEVQGAAYAARGDYYRAIEYYERALELDPNNAWAWEHLGWAYAAR |
| | GDYDRAFEYYERALELDPDDAHVWFRLGFAYAVRGDYDRAIEYYERALELDPEDELARAE |
| | LESARRARQGQAGQGGGSGLNDIFEAQKIEWHECHHHHHH |
| 101 | MGGGGSAAAGDEAARWEVQGAAYAARGDYDRAIEYYERALELDPSNAWAWEHLAWAYAAR |
| | GDYDRAIECYERALELDPDDAHAWFRLGFSYAARGDYDRAIEYYERALELDPEDELARAE |
| | LESARRARQGQAGQGGGSGLNDIFEAQKIEWHECHHHHHH |
| 102 | MGGGGSAAAGDEVARWEVQGAAYAARGDYDRAIEYYERALELDPNNAWAWEHLGWAYAAR |
| | GDYDRAIEYYERALELDPDDAHEWFRLGFAYAARGDYDRAIEYYERALELDPEDELARAE |
| | LESARRARQGQAGQGGGSGLNDIFEAQKIEWHECHHHHHH |
| 103 | MGGGGSAAAGDEADRWADLGAAYAARGDYDRAIEYYQRALELDPNNAWAWWDLGVAYAAR |
| | GDYDRAIEYYQRALELDPNNTVAWRRSGHAYAARGDYDRATEYYQRALELDPNNELAREQ |
| | LEHARRARRGQAGQGGGSGLNDIFEAQKIEWHECHHHHHH |
| 104 | MGGGGSAAAGDEADRWADLGAAYAARGDYDRAIEYYQRALEIDPNNAWAWWDLGVAYAAR |
| | GDYDRAIEYYQRALELDPNNAVAWSRLGHAYAARGDYDRAIEYYQRALELDTNNEVARTL |
| | LEQARRARRGQAGQGGGSGLNDIFEAQKIEWHECHHHHHH |
| 105 | MGGGGSAAAGDEANRWYYLGAACAARGDYDRAIEYYQRALELDPNNAWAWWDLGVAYAAR |
| | GDYDRAIEYYQRALELDPNNAVAWRRLGHAYAARGDYDRATEYYQRALELDPNNELAREQ |
| | LEHARRARRGQAGQGGGSGLNDIFEAQKIEWHECHHHHHH |
| 106 | MGGGGSAAAGDEADRWADLGAAYAARGDYDRAIEYYQRALELDPNNAWAWWDLGVAYAAR |
| | GDYDRAIEYYQRALELDPNNSVAWRRLGHAYAARGDCDRATEHYQRALELDPNNELAREQ |
| | LEHARRARRGQAGQGGGSGLNDIFEAQKIEWHECHHHHHH |
| 107 | MGGGGSAAAGDEANRWYYLGAAYAARGDYDRAIEYYQLALELDPNNAWAWWDLGVAYAAR |
| | GDYDRAIEYYQRALELDPNNAVAWRRLGHAYAARGDYDRATEYYQRALELDPNNELAREQ |
| | LEHARRARRGQAGQGGGSGLNDIFEAQKIEWHECHHHHHH |
| 108 | MGGGGSAAAGDEANRWYYLGAAYAARGDFDRAIEYYQRALELDPNNAWAWWDLGVAYAAR |
| | GDFDRAIEYYQRALELDSNNAVVWRRLGHAYAARGDYDRATEYYQRALELDPNNELAREQ |
| | LEHARRARRGQAGQGGGSGLNDIFEAQKIEWHECHHHHHH |
| 109 | MGGGGSAAAGDEADRWADLGAAYAARGYYDRAIEYYQRALELDPNNAWAWWDLGVAYAAR |
| | GDYDRAIEYYQRALELDPNNAVVWRRLGHAYAARGDYDRATEYYQRALELDPNNELAREQ |
| | LEHARRARRGQAGQGGGSGLNDIFEAQKIEWHECHHHHHH |
| 110 | MGGGGSAAAGDEADRWADLGAAYAARGDYDRAIEYYQRALELDPNNAWAWWDLGVAYAAR |
| | GDFDRAIEYYQRALELDSNNAVVWRRLGHAYAARGDYDRATEYYQRALELDPNNELAREQ |
| | LEHARRARRGQAGQGGGSGLNDIFEAQKIEWHECHHHHHH |
| 111 | MGGGGSAAAGDEADRWADLGAAYAARGDYDRAIEYYQRALELDPNNAWAWWDLGVAYAAR |
| | GDYDRAIEYYQRALELDPNDAVVWRRLGHAYAARGDYDRATEYYQRALELDPNNELAREQ |
| | LEHARRARRGQAGQGGGSGLNDIFEAQKIEWHECHHHHHH |
| 112 | MGGGGSAAAGDEADRWADLGAAYAARGDYDRAIEYYQRALELDPNNAWAWWDLGVAYAAR |
| | GDYDRAIEYYQRALELDLNNTVVWRRLGHAYAARGDYDRATEYYQRALELDPNNELAREQ |
| | LEHARRARRGQAGQGGGSGLNDIFEAQKIEWHECHHHHHH |
| 113 | MGGGGSAAAGDEADRWADLGAAYAARGDYDRAIEYYQRALELDPNNAWAWWDLGVAYAAR |
| | GDYDRAIEYYQRALELDPNNSVAWRRSGHAYAARGDYDRATEYYQRALELDPNNELAREQ |
| | LEHARRARRGQAGQGGGSGLNDIFEAQKIEWHECHHHHHH |
| 114 | MGGGGSAAAGDEADRWADLGAAYAARGDYDRAIEYYQRALELDPNNAWAWWDLGVAYAAR |
| | GDYDRAIEYYQRALELDPNNAVTWRRLGHAYDARGDYDRATEYYQRALELDPNNELAREQ |
| | LEHARRARRGQAGQGGGSGLNDIFEAQKIEWHECHHHHHH |
| 115 | MGGGGSAAAGDEADRWADLGAAYAARGDYDRAIEYYQRALELDPNNGWAWWDLGVAYAAR |
| | GDYDRAIEYYQRALELDPNNAVVWRRLGHAYAARGDYDRATEYYQRALELDPNNELAREQ |
| | LEHARRARRGQAGQGGGSGLNDIFEAQKIEWHECHHHHHH |
| 116 | MGGGGSAAAGDEADRWADLGAAYAARGDYDRAIEYYQRALELDPNNSWAWWDLGVAYAAR |
| | GDYDRAIEYYHRASELDPNNAVAWRRLGHAYAARGDYDRATEYYQRALELDPNNELAREQ |
| | LEHARRARRGQAGQGGGSGLNDIFEAQKIEWHECHHHHHH |
| 117 | MGGGGSAAAGDEANRWYYLGAAYAARGDYDSAIEYYQRALELDPNNAWAWWDLGVAYAAR |
| | GDYDRAIEYYQRALELDPNNAVAWRRLGHAYAARGDYDRATEYYQRALELDPNNELAREQ |
| | LEHARRARRGQAGQGGGSGLNDIFEAQKIEWHECHHHHHH |
| 118 | MDEALRWWDQGAAYAARGDYDRAIEYYERALELDPNNAWAWWDLGVAYAARGDYDRAIEY |
| | YERALELDPDDAHAWFRLGWAYAARGDYDRAIEYYERALELDPEDELAREELEEARRARQ |
| | GQAGQGWAEFRQRLAAIRTRLEALGGSEAELAAFEREIAAFESELQAYAGAGNPEVEALR |
| | REAAAIRDELQAYRHNHHHHHHC |
| 119 | MDEAARWEWQGAAYAARGDYDRAIEYYERALELDPNNAWAWWELGVAYAARGELDRAIEY |
| | YERALELDPDDAHAWFRLGWAYAARGDYDRAIEYYQRALELDPEDELARRILEDARRARQ |
| | GQAGQGWAEFRQRLAAIRTRLEALGGSEAELAAFEREIAAFESELQAYAGAGNPEVEALR |
| | REAAAIRDELQAYRHNHHHHHHC |
| 120 | MDEAERWYDLGAAYAARGDYDRAIEYYQRALELDPNNAWAWWELGVAYAARGDYDRAIEY |
| | YQRALELDPNNAVAWARLGIAYAARGDYDRAIEYYQRALELDPNNEVARDALEYARRARR |
| | GQAGQGWAEFRQRLAAIRTRLEALGGSEAELAAFEREIAAFESELQAYAGAGNPEVEALR |
| | REAAAIRDELQAYRHNHHHHHHC |
| 121 | MAQAAAGDEAARWEWQGAAYAARADYDRAIEYYERALELDPNNAWAWWELGVAYAARGEL |
| | DRAIEYYERALELDPDDAHAWFRLGWAYAARGDYDRAIEYYQRALELDPEDELAREELEE |
| | ARRARQGQAGQGGGSGLNDIFEAQKIEWHECHHHHHHRALELDPNNAWAWWELGVAYAAR |
| | GELDRAIEYYERALELDPDDAHAWFRLGWAYAARGDYDRAIEYYQRALELDPEDELAREE |
| | LEEARRARQGQAGQGGGSGLNDIFEAQKIEWHECHHHHHH |
| 122 | MGGGGSAAAGDEAARWEVQGAAYAARGDYDRAIEYYERALELDPNNAWAWEHLGWAYAAR |
| | GDYDRAIEYYERALELDPDDAHAWFRLGFAYAARGDYDRAIEYYERALELDPEDELARAE |
| | LESARRARQGQAGQGWAEFRQRLAAIRTRLEALGGSEAELAAFEREIAAFESELQAYAGA |
| | GNPEVEALRREAAAIRDELQAYRHNGQAGQGGGSGLNDIFEAQKIEWHECHHHHHH |
| 123 | MGGGGSAAAGDEAERWWRLGDAYRWRGDYDRAIEYYQRALELDPNNALAWVELGQAYTRR |
| | GDYDRAIEYYQRALGLDPNNAFAWHLLGEAYDERGDYDRAIEYYQRALELDPNNEWAREL |
| | LEWARRLRRGQAGQGGGSGLNDIFEAQKIEWHECHHHHHH |
| 124 | MGGGGSAEAGDEAVRWEIQGAAYAARGDYDRAIEYYERALELDPNNAWAWWELGVAYAAR |
| | GDYDRAIEYYERALELDPDDAHAWFRLGWAYAARGDYDRAIEYYERALELDPEDETARDV |
| | LEGARRARQGQAGQGGGSGLNDIFEAQKIEWHECHHHHHH |
| 125 | MGGGGSAAAGDEAERWRRLGHAYHVRGDYDRAIEYYQRALEPDPNNAYAWIELGHAYWRR |
| | GDYDRAIEYYQRALELDPNNAEAWVVLGIAYEQRGDYDRAIEYYQRALELDPNNEFARRH |
| | LEEARRERRGQAGQGGGSGLNDIFEAQKIEWHECHHHHHH |
| 126 | MDEAVRWEIQGAAYAARGDYDRAIEYYERALELDPNNAWAWWELGVAYAARGDYDRAIEY |
| | YERALELDPDDAHVWFRLGWAYATRGDYDRAIEYYERALELDPEDELARRILEDARRARQ |
| | GQAGQGWAEFRQRLAAIRTRLEALGGSEAELAAFEREIAAFESELQAYAGAGNPEVEALR |
| | REAAAIRDELQAYRHNHHHHHHC |
| 127 | MDEAVRWEIQGAAYAARGDYDRAIEYYERALELDPNNAWAWWELGVAYAARGDYDRAIEY |
| | YERALELDPDDAHVWFRLGWAYAVRGDYDRAIEYYERALELDPEDELARRILEDARRARQ |
| | GQAGQGWAEFRQRLAAIRTRLEALGGSEAELAAFEREIAAFESELQAYAGAGNPEVEALR |
| | REAAAIRDELQAYRHNHHHHHHC |
| 128 | MGDEAERWYDLGAAYAARGDFDRAIEYYRRTLELDPNDAWAWWELGVAYAARGDYDRAIE |
| | YYQRALELDPNNAVAWARLGIAYATRGDYDRAIEYYQRALELDPNNEVARDALEYARRVR |
| | RCHHHHHH |
| 129 | MGHHHHHHDEAERWYDLGAAYAARGDFDRAIEYYRRTLELDPNDAWAWWELGVAYAARGD |
| | YDRAIEYYQRALELDPNNAVAWARLGIAYATRGDYDRAIEYYQRALELDPNNEVARDALE |
| | YARRVRRC |
| 130 | MAWAEFRQRLAAIRTRLEALGGSEAELAAFEREIAAFESELQAYAGAGNPEVEALRREAA |
| | AIRDELQAYRHNDEAERWYDLGAAYAARGDFDRAIEYYRRTLELDPNDAWAWWELGVAYA |
| | ARGDYDRAIEYYQRALELDPNNAVAWARLGIAYATRGDYDRAIEYYQRALELDPNNEVAR |
| | DALEYARRVRRHHHHHH |
| 131 | MGLAEAAAREAAARAADEAERWYDLGAAYAARGDEDRAIEYYRRILELDPNDAWAWWELG |
| | VAYAARGDYDRAIEYYQRALELDPNNAVAWARLGIAYATRGDYDRAIEYYQRALELDPNN |
| | EVARDALEYARRVRRHHHHHH |
| 132 | MGDEAERWYDLGAAYAARGDFDRAIEYYRRTLELDPNDAWAWWELGVAYAARGDYDRAIE |
| | YYQRALELDPNNAVAWARLGIAYATRGDYDRAIEYYQRALELDPNNEVARDALEYARRVR |
| | RC |
| 133 | MGDEAERWYDLGAAYAARGDYDRATEFYQRALELDPNDAWAWWELGIAYAARGDYDRAIE |
| | YYQRALELDPNNAVAWARLGIAYAARGDYDRAIEYYQRALELDPNNEVARDALEYARRAR |
| | RCHHHHHH |
| 134 | MGHHHHHHDEAERWYDLGAAYAARGDYDRATEFYQRALELDPNDAWAWWELGIAYAARGD |
| | YDRAIEYYQRALELDPNNAVAWARLGIAYAARGDYDRAIEYYQRALELDPNNEVARDALE |
| | YARRARRC |
| 135 | MAWAEFRQRLAAIRTRLEALGGSEAELAAFEREIAAFESELQAYAGAGNPEVEALRREAA |
| | AIRDELQAYRHNDEAERWYDLGAAYAARGDYDRATEFYQRALELDPNDAWAWWELGIAYA |
| | ARGDYDRAIEYYQRALELDPNNAVAWARLGIAYAARGDYDRAIEYYQRALELDPNNEVAR |
| | DALEYARRARRHHHHHH |
| 136 | MGLAEAAAREAAARAADEAERWYDLGAAYAARGDYDRATEFYQRALELDPNDAWAWWELG |
| | IAYAARGDYDRAIEYYQRALELDPNNAVAWARLGIAYAARGDYDRAIEYYQRALELDPNN |
| | EVARDALEYARRARRHHHHHH |
| 137 | MGDEAERWYDLGAAYAARGDYDRATEFYQRALELDPNDAWAWWELGIAYAARGDYDRAIE |
| | YYQRALELDPNNAVAWARLGIAYAARGDYDRAIEYYQRALELDPNNEVARDALEYARRAR |
| | RC |
| 138 | MGDEAERWYDLGAAYAARGDYDRAIEYYQRALELDPNDAWAWWELGVAYAARGDYDRAIE |
| | YYQRALELDPNNAVAWARLGIAYAARGDYDRAIEYYQRALELDPNNEVARDALEYARRAR |
| | RCHHHHHH |
| 139 | MGDEAERWYDLGAAYAARGDYDRAIEYYQRALEHDPNDAWAWWELGVAYAARGDYDRAIE |
| | YYQRALELDTNNAVAWARLGIAYAARGDYDRAIEYYQRALELDPNNEVARGALEYARRAR |
| | RCHHHHHH |
| 140 | MAWAEFRQRLAAIRTRLEALGGSEAELAAFEREIAAFESELQAYAGAGNPEVEALRREAA |
| | AIRDELQAYRHNDEAERWYDLGAAYAARGDYDRAIEYYQRALEHDPNDAWAWWELGVAYA |
| | ARGDYDRAIEYYQRALELDINNAVAWARLGIAYAARGDYDRAIEYYQRALELDPNNEVAR |
| | GALEYARRARRHHHHHH |
| 141 | MGLAEAAAREAAARAADEAERWYDLGAAYAARGDYDRAIEYYQRALEHDPNDAWAWWELG |
| | VAYAARGDYDRAIEYYQRALELDTNNAVAWARLGIAYAARGDYDRAIEYYQRALELDPNN |
| | EVARGALEYARRARRHHHHHH |
| 142 | MGDEAERWYDLGAAYAARGDYDRAIEYYQRALELDPNDAWAWWELGVAYAARGDYDRAIE |
| | YYQRALELDLNNAVAWARLGIAYAARGDYDRAIEYYQRALELDPNNEVARDALEYARRAR |
| | RCHHHHHH |
| 143 | MGHHHHHHDEAERWYDLGAAYAARGDYDRAIEYYQRALEHDPNDAWAWWELGVAYAARGD |
| | YDRAIEYYQRALELDTNNAVAWARLGIAYAARGDYDRAIEYYQRALELDPNNEVARGALE |
| | YARRARRC |
| 144 | MGHHHHHHDEAERWYDLGAAYAARGDYDRAIEYYQRALELDPNDAWAWWELGVAYAARGD |
| | YDRAIEYYQRALELDLNNAVAWARLGIAYAARGDYDRAIEYYQRALELDPNNEVARDALE |
| | YARRARRC |
| 145 | MAWAEFRQRLAAIRTRLEALGGSEAELAAFEREIAAFESELQAYAGAGNPEVEALRREAA |
| | AIRDELQAYRHNDEAERWYDLGAAYAARGDYDRAIEYYQRALELDPNDAWAWWELGVAYA |
| | ARGDYDRAIEYYQRALELDLNNAVAWARLGIAYAARGDYDRAIEYYQRALELDPNNEVAR |
| | DALEYARRARRHHHHHH |
| 146 | MGLAEAAAREAAARAADEAERWYDLGAAYAARGDYDRAIEYYQRALELDPNDAWAWWELG |
| | VAYAARGDYDRAIEYYQRALELDLNNAVAWARLGIAYAARGDYDRAIEYYQRALELDPNN |
| | EVARDALEYARRARRHHHHHH |
| 147 | MGHHHHHHDEAERWYDLGAAYAARGDYDRAIEYYQRALELDPNDAWAWWELGVAYAARGD |
| | YDRAIEYYQRALELDLNNAVAWARLGIAYAARGDYDRAIEYYQRALELDPNNEVARDALE |
| | YARRARRAPKC |
| 148 | MGDEAERWYDLGAAYAARGDYDRAIEYYQRALELDPNDAWAWWELGVAYAARGDYDRAIE |
| | YYQRALELDLNNAVAWARLGIAYAARGDYDRAIEYYQRALELDPNNEVARDALEYARRAR |
| | RC |
| 149 | MDEAAHWEVQGAAYAARGDYDRAIEYYERALELDPNNAWAWEHLGWAYAARGDFDRAIEY |
| | YERALELDPDDAHAWFRLGFAYAARGDYDRAIEYYERALELDPEDELARAELESARRARQ |
| | GQAGQGWAEFRQRLAAIRTRLEALGGSEAELAAFEREIAAFESELQAYAGAGNPEVEALR |
| | REAAAIRDELQAYRHNHHHHHHC |
| 150 | MDEAARWEVQGAAYAARGDYDRAIEYYERVLELDPNDAWAWEHLGWAYAARGDYDRAIEY |
| | YERALEVDPDDAHAWFRLGFAYAARGDYDRAMEYYERALELDPEDELARAELESARRARQ |
| | GQAGQGWAEFRQRLAAIRTRLEALGGSEAELAAFEREIAAFESELQAYAGAGNPEVEALR |
| | REAAAIRDELQAYRHNHHHHHHC |
| 151 | MDEAARWEVQGAAYAARGDYDRAIEYYERALELDPNNAWAWEHLGWAYAARGDYDRAIEY |
| | YERALELDPDDAHSWFRLGFAYAVRGDYDRAIEYYERALELDPEDELARAELESARRARQ |
| | GQAGQGWAEFRQRLAAIRTRLEALGGSEAELAAFEREIAAFESELQAYAGAGNPEVEALR |
| | REAAAIRDELQAYRHNHHHHHHC |
| 152 | MDEVARWEVQGAAYAARGGYDRAIEYYERALELDPNNAWAWEHLGWAYAARGDYDRAIEY |
| | YERALELDPDDAHEWFRLGFAYAARGDYDRAIEYYERALELDPEDELARAELESARRARQ |
| | GQAGQGWAEFRQRLAAIRTRLEALGGSEAELAAFEREIAAFESELQAYAGAGNPEVEALR |
| | REAAAIRDELQAYRHNHHHHHHC |
| 153 | &&&&&&&X¹RWX²X³&&&&&&&&&&&&&&&&&&&&&&&&&&&&&X⁴AWX⁵X⁶LGX⁷&&&&&& |
| | &&&&&&&&&&&&&&&&&&&&X⁸AWX⁹X¹⁰LGX¹¹&&&&&&&&&&&&&&&&&&&&&&&&&&X¹² |
| | ARX¹³X¹⁴LEX¹⁵ |
| 154 | MGDEADRWYDQGAAYAARGDYDRAIEYYERALELDPNDAWAWWELGVAYAARGDYDRAIE |
| | YYQRALELDPNDAVAWARLGHAYAARGDYDRAIEYYQRALELDPNNEAARSNLEYARRAA |
| | ACGLNDIFEAQKIEWHEHHHHHH |
| 155 | MGDEADRWYYQGAAYAARGDYDRAIEYYERALELDPNNAWAWWELGVAYAARGDYDRAIE |
| | YYQRALELDPNDAVAWARLGHAYAARGDYDRAIEYYQRALELDPNNEAARSNLEYARRAA |
| | ACGLNDIFEAQKIEWHEHHHHHH |
| 156 | MGDEADRWYDQGAAYAARGDYDRAIEYYQRALELDPNNAWAWWELGVAYAARGDYDRAIE |
| | YYQRALELDPNDAVAWSRLGHAYAARGDYDRAIEHYQRALELDPNNEVARHLLEVARRAA |
| | ACGLNDIFEAQKIEWHEHHHHHH |
| 157 | MGDEADRWYDQGAAYAARGDYDRAIEYYQRALELDPNNAWAWWELGVAYAARGDYDRAIE |
| | YYQRALELDPNDAVAWSRLGHAYAARGDYDRAIEYYQRALELDPNNEVARHLLEVARRAA |
| | ACGLNDIFEAQKIEWHEHHHHHH |
| 158 | MGDEADRWYDQGAAYAARGDYDRAIEYYERALELDPNNAWAWWELGVAYAARGDYDRAIE |
| | YYQRALELDPNDAVAWARLGHAYAARGDYDRAIEYYQRALELDPNNEAARSNLEYARRAA |
| | ACGLNDIFEAQKIEWHEHHHHHH |
| 159 | MGDEADRWYDQGAAYAARGDYDRAIEYYQRALELDPNNAWAWWELGVAYAARGDYDRAIE |
| | YYQRALELDPNDAVAWSRLGHAYAARGDYDRAIEYYQRALELDPNNEVARHLLEVASRAA |
| | ACGLNDIFEAQKIEWHEHHHHHH |
| 160 | MGDEGDRWYYQGAAYAARGDYDRAIEYYERALELDPNNAWAWWELGVAYAARGDYDRAIE |
| | YYQRALELDPNNTVAWARLGHAYAARGDYDRAIEYYQRALELDPNNEAARSNLEYSPPAA |
| | ACGLNDIFEAQKIEWHEHHHHHH |
| 161 | MGDEADRWYYQGAAYAARGDYDRALEYYERALELDPSNAWAWWELGVAYAARGDYDRAIE |
| | YYQRALELDPNDAVAWARLGHAYAARGDYDRAIEYYQRALELDPNNEAARSNLEYARRAA |
| | ACGLNDIFEAQKIEWHEHHHHHH |
| 162 | MGDEADRWYYQGAAYAARGDYDRAIEYYERALELDPNNAWAWWELGVAYAARGDYDRAIE |
| | YYQRALELDPNNTVAWARLGHAYAARGDYDRAIEYYQRALELDPNNEAARSNLEYARRAA |
| | ACGLNDIFEAQKIEWHEHHHHHH |
| 163 | MGDEADRWYYRGAAYAARGDYDRAIEYYERALELDPNNAWAWWELGVAYAARGDYDRAIE |
| | YYQRALELDPNNTVAWARLGHAYAARGDYDRAIEYYQRALELDPNNEAARSNLEYARRAA |
| | ACGLNDIFEAQKIEWHEHHHHHH |
| 164 | MGDEADRWYDQGAAYAARGDYDRAIEYYERALELDPNNAWAWWELGAAYAARGDYDRAIE |
| | YYQRALELDPNNAVAWSRLGHAYAARGDYDRAIEYYQRALELDPNNEVARHLLEVARRAA |
| | ACGLNDIFEAQKIEWHEHHHHHH |
| 165 | MGDEAERWYDLGAAFAARGDYDRAIEYYQRALELDPNDAWAWWELGVAYAARGDYDRAIE |
| | YYQRALELDPNNAVAWFRLGWAYAERGDYDRAIEYYQRALELDPNNELARSELENARRAA |
| | ACGLNDIFEAQKIEWHEHHHHHH |
| 166 | MGDEAERWYDLGAAYAARGDYDRAIEYYQRALELDPNDAWAWWELGVAYAARGDYDRAIE |
| | YYQRALELDPNNAVAWFRLGWAYAARGDYDRAIEYYQRALELDPNNELARSELENARRAA |
| | ACGLNDIFEAQKIEWHEHHHHHH |
| 167 | MGDEAERWYDLGAAYAARGDYDRAIEYYQRALELDPNDAWAWWELGVAYAARGDYDRAIE |
| | YYQRALELDPNNAVAWFRLGWAYAERGDYDRAIEYYQRALELDPNNELARSELENARRAA |
| | ACGLNDIFEAQKIEWHEHHHHHH |
| 168 | MGDEAERWYDLGAAYAARGDYDRAIEYYQRALELDPNNAWAWWELGVAYAARGDYDRAIE |
| | YYQRALELDPNDAVAWFRLGWAYAARGDYDRAIEYYQRALELDPNNELARSELVNARRAA |
| | ACGLNDIFEAQKIEWHEHHHHHH |
| 169 | MGDEAERWYDLGXAYAARGDYDRAIEYYQRALELDPNDAWAWWELGVAYAARGDYDRAIE |
| | YYQRALELDPNNAVAWERLGWAYAERGDYDRAIEYYQRALELDPNNELARSELENARRAA |
| | ACGLNDIFEAQKIEWHEHHHHHH |
| 170 | MGDEAERWYDLGAAYAARGDFDRAIEYYQRALELDPNDAWAWWELGVAYAARGDYDRAIE |
| | YYQRALELDPNNAVAWFRLGWAYAERGDYDRAIEYYQRALELDPNNELARSELENARRAA |
| | ACGLNDIFEAQKIEWHEHHHHHH |
| 171 | MGDEAERWYDLGAAYAARGDYDRAIEYYQRALELDPNDAWAWWELGVAYAARGDYDRAIE |
| | YYQRTLELDPNNAVAWFRLGWAYAERGDYDRAIEYYQRALELDPNNELARSELENARRAA |
| | ACGLNDIFEAQKIEWHEHHHHHH |
| 172 | MGDEAERWYDLGAAYAARGDYDRAIEYYQRALELDPNNAWAWWELGVAYAARGDYDRAIE |
| | YYQRALELDPNDAVAWFRLGWAYAARGDYDRAIEYYQRALELDPNNELARSELENARRAA |
| | ACGLNDIFEAQKIEWHEHHHHHH |
| 173 | MGDEAERWYDLGAAYAARGDYDRAIEYYQRALELDPNDAWAWWELGVAYAARGDYDRAIE |
| | YYQRALELDPNNAVAWFRLGWAYAARGDYDRAIEYYQRALELDPNNELARSELVNARRAA |
| | ACGLNDIFEAQKIEWHEHHHHHH |
| 174 | MGDEASRWYHLGVAYAARGDYDRAIEYYQRALELDPNNAWAWWELGVAYAARGDYDRAIE |
| | YYQRALELDPNNAIAWSRLGHAYAARGDYDRAIEYYQRALELDPNNEVARHLLEYARRAA |
| | ACGLNDIFEAQKIEWHEHHHHHH |
| 175 | MGDEADRWYDQGAAYAARGDYDRAIEYYQRALELDPNNAWAWWELGVAYAARGDYDRAIE |
| | YYQRALELDPNDAVAWSRLGHAYAARGDYDRAIEYYQRALELDPNNEVARHLLEVARRLE |
| | DEADRWYDQGAAYAARGDYDRAIEYYQRALELDPNNAWAWWELGVAYAARGDYDRAIEYY |
| | QRALELDPNDAVAWSRLGHAYAARGDYDRAIEYYQRALELDPNNEVARHLLEVYRRAAAH |
| | HHHHHC |
| 176 | MGDEADRWYDQGAAYAARGDYDRAIEYYQRALELDPNNAWAWWELGVAYAARGDYDRAIE |
| | YYQRALELDPNDAVAWSRLGHAYAARGDYDRAIEYYQRALELDPNNEVARHLLEVARRAA |
| | AHHHHHHC |
| 177 | MGDEADRWYDQGAAYAARGDYDRAIEYYQRALELDPNNAWAWWELGVAYAARGDYDRAIE |
| | YYQRALELDPNDAVAWSRLGHAYAARGDYDRAIEYYQRALELDPNNEVARHLLEVARRLE |
| | GGGGSDEADRWYDQGAAYAARGDYDRAIEYYQRALELDPNNAWAWWELGVAYAARGDYDR |
| | AIEYYQRALELDPNDAVAWSRLGHAYAARGDYDRAIEYYQRALELDPNNEVARHLLEVAR |
| | RAAAHHHHHHC |
| 178 | MAQHDKIQQAADKEILHLPNLTEEQRNKFRQSLRDDPSVSAEILAEAKKLNDAQAPKAMG |
| | DEADRWYDQGAAYAARGDYDRAIEYYQRALELDPNNAWAWWELGVAYAARGDYDRAIEYY |
| | QRALELDPNDAVAWSRLGHAYAARGDYDRAIEYYQRALELDPNNEVARHLLEVARRAAAH |
| | HHHHHC |
| 179 | MAQHDKIQQAADKEILHLPNLTEEQRNKFRQSLRDDPSVSAEILAEAKKLNDAQAPKAQH |
| | DKIQQAADKEILHLPNLTEEQRNKFRQSLRDDPSVSAEILAEAKKLNDAQAPKAMGDEAD |
| | RWYDQGAAYAARGDYDRAIEYYQRALELDPNNAWAWWELGVAYAARGDYDRAIEYYQRAL |
| | ELDPNDAVAWSRLGHAYAARGDYDRAIEYYQRALELDPNNEVARHLLEVARRAAAHHHHH |
| | HC |
| 180 | MGDEADRWYDQGAAYAARGDYDRAIEYYQRALELDPNNAWAWWELGVAYAARGDYDRAIE |
| | YYQRALELDPNDAVAWSRLGHAYAARGDYDRAIEYYQRALELDPNNEVARHLLEVARRAA |
| | AAQHDKIQQAADKEILHLPNLTEEQRNKFRQSLRDDPSVSAEILAEAKKLNDAQAPKHHH |
| | HHHC |
| 181 | MGDEADRWYDQGAAYAARGDYDRAIEYYQRALELDPNNAWAWWELGVAYAARGDYDRAIE |
| | YYQRALELDPNDAVAWSRLGHAYAARGDYDRAIEYYQRALELDPNNEVARHLLEVARRAA |
| | AAQHDKIQQAADKEILHLPNLTEEQRNKFRQSLRDDPSVSAEILAEAKKLNDAQAPKAQH |
| | DKIQQAADKEILHLPNLTEEQRNKFRQSLRDDPSVSAEILAEAKKLNDAQAPKHHHHHHC |
| 182 | MGDEADRWYDQGAAYAARGDYDRAIEYYQRALELDPNNAWAWWELGVAYAARGDYDRAIE |
| | YYQRALELDPNDAVAWSRLGHAYAARGDYDRAIEYYQRALELDPNNEVARHLLEVARRAA |
| | ADEADRWYDQGAAYAARGDYDRAIEYYQRALELDPNNAWAWWELGVAYAARGDYDRAIEY |
| | YQRALELDPNDAVAWSRLGHAYAARGDYDRAIEYYQRALELDPNNEVARHLLEVARRAAA |
| | GLNDIFEAQKIEWHEHHHHHHC |
| 183 | MGDEADRWYDQGAAYAARGDYDRAIEYYQRALELDPNNAWAWWELGVAYAARGDYDRAIE |
| | YYQRALELDPNDAVAWSRLGHAYAARGDYDRAIEYYQRALELDPNNEVARHLLEVARRAA |
| | ADEADRWYDQGAAYAARGDYDRAIEYYQRALELDPNNAWAWWELGVAYAARGDYDRAIEY |
| | YQRALELDPNDAVAWSRLGHAYAARGDYDRAIEYYQRALELDPNNEVARHLLEVARRAMG |
| | DEADRWYDQGAAYAARGDYDRAIEYYQRALELDPNNAWAWWELGVAYAARGDYDRAIEYY |
| | QRALELDPNDAVAWSRLGHAYAARGDYDRAIEYYQRALELDPNNEVARHLLEVARRAAAD |
| | EADRWYDQGAAYAARGDYDRAIEYYQRALELDPNNAWAWWELGVAYAARGDYDRAIEYYQ |
| | RALELDPNDAVAWSRLGHAYAARGDYDRAIEYYQRALELDPNNEVARHLLEVARRAAAGL |
| | NDIFEAQKIEWHEHHHHHHC |
| 184 | MADEADRWYDQGAAYAARGDYDRAIEYYQRALELDPNNAWAWWELGVAYAARGDYDRAIE |
| | YYQRALELDPNDAVAWSRLGHAYAARGDYDRAIEYYQRALELDPNNEVARHLLEVAQRAA |
| | AAQHDKIQQAADKEILHLPNLTEEQRNKFRQSLRDDPSVSAEILAEAKKLNDAQAPKC |
| 185 | MADEADRWYDQGAAYAARGDYDRAIEYYQRALELDPNNAWAWWELGVAYAARGDYDRAIE |
| | YYQRALELDPNDAVAWSRLGHAYAARGDYDRAIEYYQRALELDPNNEVARHLLEVAQAPK |
| | VDAKFDKEQQAARAEILHLPNLTEEQRNKFIQSLKDDPSQSANLLAEAKKLNDAQAPKVD |
| | AKFDKEQQAARAEILHLPNLTEEQRNKFIQSLKDDPSQSANLLAEAKKLNDAQAPKVDAK |
| | FDKEQQAARAEILHLPNLTEEQRNKFIQSLKDDPSQSANLLAEAKKLNDAQAPKC |
| 186 | MAQHDKIQQAADKEILHLPNLTEEQRNKFRQSLRDDPSVSAEILAEAKKLNDAQAPKADE |
| | ADRWYDQGAAYAARGDYDRAIEYYQRALELDPNNAWAWWELGVAYAARGDYDRAIEYYQR |
| | ALELDPNDAVAWSRLGHAYAARGDYDRAIEYYQRALELDPNNEVARHLLEVAQAPKAQHD |
| | KIQQAADKEILHLPNLTEEQRNKFRQSLRDDPSVSAEILAEAKKLNDAQAPKIAAQHDKI |
| | QQAADKEILHLPNLTEEQRNKFRQSLRDDPSVSAEILAEAKKLNDAQAPKC |
| 187 | MADEADRWYDQGAAYAARGDYDRAIEYYQRALELDPNNAWAWWELGVAYAARGDYDRAIE |
| | YYQRALELDPNDAVAWSRLGHAYAARGDYDRAIEYYQRALELDPNNEVARHLLEVAQRAA |
| | AAQHDKIQQAADKEILHLPNLTEEQRNKFRQSLRDDPSVSAEILAEAKKLNDAQAPKAQH |
| | DKIQQAADKEILHLPNLTEEQRNKFRQSLRDDPSVSAEILAEAKKLNDAQAPKC |
| 188 | MGDEADRWYDQGAAYAARGDYDRAIEYYQRALELDPNNAWAWWELGVAYAARGDYDRAIE |
| | YYQRALELDPNDAVAWSRLGHAYAARGDYDRAIEHYQRALELDPNNEVARHLLEVARRAA |
| | AHHHHHHC |
| 189 | MGDEADRWYDQGAAYAARGDYDRAIEYYQRALELDPNNAWAWWELGVAYAARGDYDRAIE |
| | YYQRALELDPNDAVAWSRLGHAYAARGDYDRAIEHYQRALELDPNNEVARHLLEVARRLE |
| | DEADRWYDQGAAYAARGDYDRAIEYYQRALELDPNNAWAWWELGVAYAARGDYDRAIEYY |
| | QRALELDPNDAVAWSRLGHAYAARGDYDRAIEHYQRALELDPNNEVARHLLEVSRRAAAH |
| | HHHHHC |
| 190 | MGDEADRWYYQGAAYAARGDYDRAIEYYERALELDPNNAWAWWELGVAYAARGDYDRAIE |
| | YYQRALELDPNDAVAWARLGHAYAARGDYDRAIEYYQRALELDPNNEAARSNLEYARRAA |
| | AHHHHHHC |
| 191 | MGDEADRWYYQGAAYAARGDYDRAIEYYERALELDPNNAWAWWELGVAYAARGDYDRAIE |
| | YYQRALELDPNDAVAWARLGHAYAARGDYDRAIEYYQRALELDPNNEAARSNLEYARRLE |
| | DEADRWYYQGAAYAARGDYDRAIEYYERALELDPNNAWAWWELGVAYAARGDYDRAIEYY |
| | QRALELDPNDAVAWARLGHAYAARGDYDRAIEYYQRALELDPNNEAARSNLEYARRAAAH |
| | HHHHRC |
| 192 | MAQHDKIQQAADKEILHLPNLTEEQRNKFRQSLRDDPSVSAEILAEAKKLNDAQAPKDEA |
| | DRWYYQGAAYAARGDYDRAIEYYERALELDPNNAWAWWELGVAYAARGDYDRAIEYYQRA |
| | LELDPNDAVAWARLGHAYAARGDYDRAIEYYQRALELDPNNEAARSNLEYARRAAAHHHH |
| | HHC |
| 193 | MAQHDKIQQAADKEILHLPNLTEEQRNKFRQSLRDDPSVSAEILAEAKKLNDAQAPKDEA |
| | DRWYYQGAAYAARGDYDRAIEYYERALELDPNNAWAWWELGVAYAARGDYDRAIEYYQRA |
| | LELDPNDAVAWARLGHAYAARGDYDRAIEYYQRALELDPNNEAARSNLEYARRAAAQHDK |
| | IQQAADKEILHLPNLTEEQRNKFRQSLRDDPSVSAEILAEAKKLNDAQAPKHHHHHHC |
| 194 | MGDEADRWYDQGAAYAARGDYDRAIEYYERALELDPNDAWAWWELGVAYAARGDYDRAIE |
| | YYQRALELDPNDAVAWARLGHAYAARGDYDRAIEYYQRALELDPNNEAARSNLEYARRAA |
| | AHHHHHHC |
| 195 | MAQHDKIQQAADKEILHLPNLTEEQRNKFRQSLRDDPSVSAEILAEAKKLNDAQAPKDEA |
| | DRWYDQGAAYAARGDYDRAIEYYERALELDPNDAWAWWELGVAYAARGDYDRAIEYYQRA |
| | LELDPNDAVAWARLGHAYAARGDYDRAIEYYQRALELDPNNEAARSNLEYARRAAAHHHH |
| | HHC |
| 196 | MGDEADRWYDQGAAYAARGDYDRAIEYYERALELDPNDAWAWWELGVAYAARGDYDRAIE |
| | YYQRALELDPNDAVAWARLGHAYAARGDYDRAIEYYQRALELDPNNEAARANLEYARRAA |
| | AGLNDIFEAQKIEWHEHHHHHHC |
| 197 | MGDEADRWYDQGAAYAARGDYDRAIEYYERALELDPNDAWAWWELGVAYAARGDYDRAIE |
| | YYQRALELDPNDAVAWARLGHAYAARGDYDRAIEYYQRALELDPNNEAARSALEYARRAA |
| | AGLNDIFEAQKIEWHEHHHHHHC |
| 198 | MGDEADRWYDQGAAYAARGDYDRAIEYYERALELDPNDAWAWWELGVAYAARGDYDRAIE |
| | YYQRALELDPNDAVAWARLGHAYAARGDYDRAIEYYQRALELDPNNEAARSNLEAARRAA |
| | AGLNDIFEAQKIEWHEHHHHHHC |
| 199 | MGDEADRWYDQGAAYAARGDYDRAIEYYERALELDPNDAWAWWELGVAYAARGDYDRAIE |
| | YYQRALELDPNDAVAWARLGHAYAARGDYDRAIEYYQRALELDPNNEAARAALEYARRAA |
| | AGLNDIFEAQKIEWHEHHHHHHC |
| 200 | MGDEADRWYDQGAAYAARGDYDRAIEYYERALELDPNDAWAWWELGVAYAARGDYDRAIE |
| | YYQRALELDPNDAVAWARLGHAYAARGDYDRAIEYYQRALELDPNNEAARANLEAARRAA |
| | AGLNDIFEAQKIEWHEHHHHHHC |
| 201 | MGDEADRWYDQGAAYAARGDYDRAIEYYERALELDPNDAWAWWELGVAYAARGDYDRAIE |
| | YYQRALELDPNDAVAWARLGHAYAARGDYDRAIEYYQRALELDPNNEAARSALEAARRAA |
| | AGLNDIFEAQKIEWHEHHHHHHC |
| 202 | MGDEADRWYDQGAAYAARGDYDRAIEYYERALELDPNDAWAWWELGVAYAARGDYDRAIE |
| | YYQRALELDPNDAVAWARLGHAYAARGDYDRAIEYYQRALELDPNNEAARAALEAARRAA |
| | AGLNDIFEAQKIEWHEHHHHHHC |
| 203 | MGDEADRWYDQGAAYAARGDYDRAIEYYERALELDPNNAWAWWELGVAYAARGDYDRAIE |
| | YYQRALELDPNNAVAWARLGHAYAARGDYDRAIEYYQRALELDPNNEAARSNLEYARRAA |
| | ACGLNDIFEAQKIEWHEHHHHHH |
| 204 | MGDEADRWYDQGAAYAARGDYDRAIEYYERALELDPNNAWAWWELGTAYAARGDYDRAIE |
| | YYQRALELDPNNAVAWSRLGHAYAARGDYDRAIEYYQRALELDPNNEVARHLLEVARRAA |
| | ACGLNDIFEAQKIEWHEHHHHHH |

1. An affinity ligand comprising an amino acid sequence represented by the formula from N-terminus to C-terminus
[A]-AERWYDLGAAYAARGDX₁₇ₐDRAX₂₁ₐEX₂₃ₐYX₂₅ₐRX₂₇ₐLEX₃₀ₐ
DPND-AWAWWELGX_{9b}AYAARGDYDRAIEYYQRALELDX_{32b}NN-
AVAWARLGIAYAX_{13c}RGDYDRAIEYYQRALELDPNN-
EVARX₅ₐALEYARRX_{13d}-[B]
wherein
X₁₇ₐ is F or Y; X₂₁ₐ is I or T; X₂₃ₐ is Y or F; X₂₅ₐ is R or Q; X₂₇ₐ is T or A; and
X₃₀ₐ is L or H;
wherein
X_{9b} is V or I; and X_{32b} is L, P or T;
wherein
X_{13c} is T or A;
wherein
X_{5d} is D or G; and X_{13d} is A or V; and
wherein
[A] is present or absent, and when present comprises M, MDE, MGGGGSAAAGDE, MGDE, MGHHHHHHDE, MGLAEAAAREAAARAADE, or MAWAEFRQRLAAIRTRLEALGGSEAELAAFEREIAAFESELQAYAGAGNPEVEALRREA AAIRDELQAYRHNDE; and
[B] is present or absent, and when present comprises a cysteine, RRC, RRCHHHHHH, or RRGQAGQGGGSGLNDIFEAQKIEWHECHHHHHH.

2. The affinity ligand of claim 1, wherein X₁₇ₐ is F; X₂₁ₐ is I; X₂₃ₐ is Y; X₂₅ₐ is R; X₂₇ₐ is T; X₃₀ₐ is L; X_{9b} is V; X_{32b} is P; X_{13c} is T; X_{5d} is D; and X_{13d} is V.

3. The affinity ligand of claim 1, wherein X₁₇ₐ is Y; X₂₁ₐ is T; X₂₃ₐ is F; X₂₅ₐ is Q; X₂₇ₐ is A; X₃₀ₐ is L; X_{9b} is I; X_{32b} is P; X_{13c} is A; X_{5d} is D; and X_{13d} is A.

4. The affinity ligand of claim 1, wherein X₁₇ₐ is Y; X₂₁ₐ is I; X₂₃ₐ is Y; X₂₅ₐ is Q; X₂₇ₐ is A; X₃₀ₐ is H; X_{9b} is V; X_{32b} is T; X_{13c} is A; X_{5d} is G; and X_{13d} is A.

5. The affinity ligand of claim 1, wherein X₁₇ₐ is Y; X₂₁ₐ is I; X₂₃ₐ is Y; X₂₅ₐ is Q; X₂₇ₐ is A; X₃₀ₐ is L; X_{9b} is V; X_{32b} is L; X_{13c} is A; X_{5d} is D; and X_{13d} is A.

6. The affinity ligand of claim 1, wherein [A] is MGHHHHHHDE and [B] is RRC.

7. The affinity ligand of claim 1, wherein [A] is MGLAEAAAREAAARAADE and [B] is RRCHHHHHH.

8. The affinity ligand of claim 2, wherein said ligand comprises any one of SEQ ID NOS: 89, 128, 130, 131 or 132.

9. The affinity ligand of claim 3, wherein said ligand comprises any one of SEQ ID NOS: 90, 133, 135, 136 or 137.

10. The affinity ligand of claim 4, wherein said ligand comprises any one of SEQ ID NOS: 139, 140, 141 or 143.

11. The affinity ligand of claim 5, wherein said ligand comprises any one of SEQ ID NOS: 86, 91, 138, 142, 144, 145, 146, 147 or 148.

12. The affinity ligand of claim 1, wherein said ligand comprises any one of SEQ ID NOS: 129, 134, 141 or 144.

13. The affinity ligand of claim 1, which further comprises a C-terminal cysteine or lysine.

14. A multimer comprising a plurality of affinity ligands according to claim 1.

15. The multimer of claim 14, which is a dimer, trimer, tetramer, pentamer, hexamer, heptamer, octamer or nonamer.

16. The affinity ligand of claim 1 or multimer thereof, wherein said ligand or multimer further comprises at least one heterologous agent operably linked to said affinity ligand to thereby form a conjugate or a fusion protein.

17. The affinity ligand or multimer of claim 16, wherein said heterologous agent is selected from the group consisting of one or more small molecule diagnostic or therapeutic agents, a peptide tag, a DNA, RNA, or hybrid DNA-RNA molecule, a traceable marker, a radioactive agent, an antibody, a single chain variable domain, and an immunoglobulin fragment.

18. A separation matrix comprising at least one affinity ligand of claim 1, or multimer thereof, coupled to a solid support.

19. (canceled)

20. The separation matrix of claim 18, wherein the affinity ligands or multimers are coupled to the solid support via thiol linkages.

21. The separation matrix of claim 18, wherein the solid support is a chromatography resin or matrix.

22. The separation matrix of claim 21, wherein the solid support is a cross-linked agarose matrix.

23. A method of isolating an antibody comprising a CH1 domain, or a fragment thereof comprising a CH1 domain, which comprises contacting said antibody or said fragment with a separation matrix of claim 18 and recovering said antibody or fragment thereof.

24. The method of claim 23, which comprises (a) contacting a separation matrix of claim 18 with a composition comprising said antibody or said fragment, (b) washing said separation matrix with a washing buffer, (c) eluting said antibody or said fragment from the separation matrix with an elution buffer, and (d) recovering said antibody or said fragment.

25. The method of claim 23, which further comprises (e) treating said separation matrix with an alkaline cleaning solution for a time sufficient to clean said matrix of residual material and to regenerate at least 80% of the antibody- or the antibody fragment-binding capacity of said separation matrix.

26. The method of claim 25, wherein the alkaline cleaning solution comprises from about 0.1 M NaOH to about 0.5 M NaOH.

27. The method of claim 25, wherein said separation matrix retains at least 80% of its antibody- or the antibody fragment-binding capacity when steps (a)-(e) are repeated at least 10 times.

28. The method of claim 25, wherein steps (a)-(e) are repeated at least 10 times with a single batch of separation matrix.

29. A nucleic acid or vector encoding an affinity ligand of claim 1 or a multimer thereof.

30. An expression vector comprising the nucleic acid or vector of claim 29, wherein the coding region of said affinity ligand is operably linked to one or more expression control elements.

31. A host cell comprising the nucleic acid or vector of claim 29.

32. The host cell of claim 31, wherein host cell is E. coli or P. pastoris.

33. A method of making a separation matrix comprising conjugating a ligand according to claim 1, or a multimer thereof, to a solid surface.

34. An affinity agent comprising a ligand comprising a TPR ligand comprising 7 helices wherein
a first helix comprises the sequence X¹RWX²X³ (SEQ ID No:1), wherein
X¹ is A, D, E, G, N, Q, S or V;
X² is A, E, W or Y; and
X³ is D, G, H, I, N, T, V, W or Y;
a third helix comprises the sequence X⁴AWX⁵X⁶LGX⁷ (SEQ ID No:2), wherein
X⁴ is W;
X⁵ is E or W;
X⁶ is D, E or H; and
X⁷ is T, V, W;
a fifth helix comprises the sequence X⁸AWX⁹X¹⁰LGX¹¹ (SEQ ID No:3), wherein
X⁸ is A, H, I, T or V;
X⁹ is A, F, N, S or R;
X¹⁰ is R; and
X¹¹ is F, I, H or W;
a seventh helix comprises the sequence X¹²ARX¹³X¹⁴LEX⁵ (SEQ ID No:4), wherein
X¹² is A, L, T or V;
X¹³ is A, D, E, H, Q, R, S or Y;
X¹⁴ is A, E, I, L, N or Q; and
X¹⁵ is A, D, E, H, NR, S, V or Y.

35. The affinity agent of claim 34 that binds to monoclonal antibodies, antibody fragments and antibody conjugates containing a CH1 domain.

36. An affinity agent comprising a ligand that binds monoclonal antibodies, antibody fragments and antibody conjugates containing a CH1 domain comprising a ligand comprising at least one sequence of SEQ ID Nos: 6-152 and 154-204, or an amino acid sequence that differs by no more than three, by no more than two, or by no more than one, substitutions, additions, or deletions.

37. The affinity agent of claim 34, wherein the TPR ligand is a multimer comprising at least two subunits.

38. The affinity agent of claim 37 wherein the sub-units are not all the same.

39. The affinity agent of claim 34, wherein the ligand is attached to a solid surface.

40. The affinity agent of claim 39 wherein the solid surface is a resin or bead.

41. The affinity agent of claim 39 wherein the solid surface is a membrane.

42. The affinity agent of claim 39 wherein the solid surface is a monolith.

43. The affinity agent of claim 39 wherein the ligand is conjugated to the solid surface via a linker.

44. (canceled)

45. A method of making an affinity agent comprising conjugating a ligand according to claim 34 to a solid surface.

46. A method of isolating a monoclonal antibody, antibody fragment, or antibody conjugate containing a CH1 domain, comprising contacting the antibody, fragment or conjugate with the affinity agent of claim 34.


SEQUENCE LISTING (231 sequences)

SEQ ID NO: 1 - protein, 8 aa, synthetic construct
  VARIANT 4: note = E or W
  VARIANT 5: note = D, E or H
  VARIANT 8: note = T, V, W
WAWXXLGX

SEQ ID NO: 2 - protein, 8 aa, synthetic construct
  VARIANT 8: note = I or V
WAWWELGX

SEQ ID NO: 3 - protein, 8 aa, synthetic construct
  VARIANT 1: note = F, or Y
  VARIANT 8: note = N or S
XAWDELGX

SEQ ID NO: 4 - protein, 8 aa, synthetic construct
WAWEHLGW

SEQ ID NO: 5 - protein, 8 aa, synthetic construct
  VARIANT 4: note = E or W
  VARIANT 5: note = D, E or H
  VARIANT 8: note = L, V or W
WAWXXLGX

SEQ ID NO: 6 - protein, 8 aa, synthetic construct
  VARIANT 8: note = V, T or A
WAWWELGX

SEQ ID NO: 7 - protein, 8 aa, synthetic construct
  VARIANT 1: note = A, H, I, T or V
  VARIANT 4: note = A, F, N, S or R
  VARIANT 8: note = F, I, H or W
XAWXRLGX

SEQ ID NO: 8 - protein, 8 aa, synthetic construct
  VARIANT 1: note = H, T or V
  VARIANT 4: note = A, F or S
  VARIANT 8: note = H, I, or W
XAWXRLGX

SEQ ID NO: 9 - protein, 8 aa, synthetic construct
  VARIANT 4: note = N, or S
  VARIANT 8: note = H, or Q
VAWXRLGX

SEQ ID NO: 10 - protein, 8 aa, synthetic construct
HAWFRLGF

SEQ ID NO: 11 - protein, 8 aa, synthetic construct
  VARIANT 1: note = H, R, or V
  VARIANT 8: note = F or W
XAWFRLGX

SEQ ID NO: 12 - protein, 8 aa, synthetic construct
  VARIANT 1: note = V or I
  VARIANT 4: note = A, F or S
  VARIANT 8: note = H or W
XAWXRLGX

SEQ ID NO: 13 - protein, 8 aa, synthetic construct
  VARIANT 1: note = A, L, T or V
  VARIANT 4: note = A, D, E, H, Q, R, S or Y
  VARIANT 5: note = A, E, I, L, N or Q
  VARIANT 8: note = A, D, E, H, N, R, S, V or Y
XARXXLEX

SEQ ID NO: 14 - protein, 8 aa, synthetic construct
  VARIANT 1: note = A, L, T or V
  VARIANT 4: note = D, H, R or Y
  VARIANT 5: note = A, I, V or L
  VARIANT 8: note = D, H, V or Y
XARXXLEX

SEQ ID NO: 15 - protein, 8 aa, synthetic construct
  VARIANT 1: note = T or V
  VARIANT 4: note = H or T
  VARIANT 8: note = Q or W
XARXLLEX

SEQ ID NO: 16 - protein, 8 aa, synthetic construct
  VARIANT 1: note = L or Q
  VARIANT 4: note = A or E
  VARIANT 5: note = E or Q
  VARIANT 8: note = S or H
XARXXLEX

SEQ ID NO: 17 - protein, 8 aa, synthetic construct
  VARIANT 1: note = L, or T
  VARIANT 4: note = A, D, E, or R
  VARIANT 5: note = E, I, Q, or V
  VARIANT 8: note = D, E, G, H, S or N
XARXXLEX

SEQ ID NO: 18 - protein, 8 aa, synthetic construct
  VARIANT 1: note = A, L or V
  VARIANT 4: note = A, H, or S
  VARIANT 5: note = A, E, L or N
  VARIANT 8: note = A, N, V or Y
XARXXLEX

SEQ ID NO: 19 - protein, 8 aa, synthetic construct
  VARIANT 1: note = L, T or V
  VARIANT 4: note = D, R, or Y
  VARIANT 5: note = A, I or L
  VARIANT 8: note = D, H or Y
XARXXLEX

SEQ ID NO: 20 - protein, 110 aa, synthetic construct
  VARIANT 1: note = Q, D, A, G, E, V, N
  VARIANT 4: note = E, A, Y, Q
  VARIANT 5: note = D, N, G, V, I, T, W, Y
  VARIANT 38: note = W, E
  VARIANT 39: note = E, D, H
  VARIANT 42: note = V, W, T
  VARIANT 69: note = V, H, T, A, I
  VARIANT 72: note = F, R, S, A, N
  VARIANT 76: note = H, W, F, I
  VARIANT 103: note = L, T, V
  VARIANT 106: note = E, Y, R, A, D, S, Q
  VARIANT 107: note = Q, L, E, I, A
  VARIANT 110: note = H, D, Y, S, E, or R
XRWXXLGAAYAARGDYDRAIEYYQRALELDPNNAWAWXXLGXAYAARGDYDRAIEYYQRALELDPNNAXAWXRLGXAYAARGDYDRAIEYYQRALELDPNNEXARXXLEX

SEQ ID NO: 21 - protein, 110 aa, synthetic construct; identical to SEQ ID NO: 20
  VARIANT 1: note = A, D, E, G, N, Q or V
  VARIANT 4: note = A, E, W or Y
  VARIANT 5: note = D, G, I, N, T, V, W or Y
  VARIANT 38: note = E or W
  VARIANT 39: note = D, E or H
  VARIANT 42: note = T, V, W
  VARIANT 69: note = A, H, I, T or V
  VARIANT 72: note = A, F, N, S or R
  VARIANT 76: note = F, I, H or W
  VARIANT 103: note = L, T or V
  VARIANT 106: note = A, D, E, Q, R, S or Y
  VARIANT 107: note = A, E, I, L or Q
  VARIANT 110: note = D, E, H, R, S or Y

SEQ ID NO: 22 - protein, 157 aa, synthetic construct
MAQAAAGDEADRWADLGAAYAARGDYDRAIEYYERALELDPNNAWAWWDLGVAYAARGDYDRAIEYYERALELDPDDATAWSRLGHAYAARGDYDRAIEYYERALELDPEDETARYLLEHARRARQGQAGQGGGSGLNDIFEAQKIEWHECHHHHHH

SEQ ID NO: 23 - protein, 157 aa, synthetic construct
MAQAAAGDEADRWADLGAAYAARGDYDRAIEYYQRALELDPNNAWAWWDLGVAYAARGDYDRAIEYYQRALELDPNNATAWNRLGHAYAARGDYDRAIEYYERALELDPEDETARYLLEHARRARQGQAGQGGGSGLNDIFEAQKIEWHECHHHHHH

SEQ ID NO: 24 - protein, 157 aa, synthetic construct
MAQAAAGDEAGRWEGQGAAYAARGDYDRAIEYYQRALELDPNNAWAWWELGVAYAARGDYDRAIEYYQRALELDPNNATAWNRLGHAYAARGDYDRAIEYYQRALELDPNNEVARSQLEHARRARRGQAGQGGGSGLNDIFEAQKIEWHECHHHHHH

SEQ ID NO: 25 - protein, 157 aa, synthetic construct
MAQAAAGDEAGRWEGQGAAYAARGDYDRAIEYYERALELDPNNAWAWWDLGVAYAARGDYDRAIEYYERALELDPDDATAWSRLGHAYAARGDYDRAIEYYERALELDPEDETARYLLEHARRARQGQAGQGGGSGLNDIFEAQKIEWHECHHHHHH

SEQ ID NO: 26 - protein, 157 aa, synthetic construct
MAQAAAGDEAGRWENQGAAYAARGDYDRAIEYYERALELDPNNAWAWWELGVAYAARGDYDRAIEYYERALELDPDDAHAWRRLGFAYAARGDYDRAIEYYERALELDPEDEVAREVLEEARRARQGQAGQGGGSGLNDIFEAQKIEWHECHHHHHH

SEQ ID NO: 27 - protein, 157 aa, synthetic construct
MAQAAAGDEAARWEWQGAAYAARADYDRAIEYYERALELDPNNAWAWWELGVAYAARGELDRAIEYYERALELDPDDAHAWFRLGWAYAARGDYDRAIEYYQRALELDPEDELAREELEEARRARQGQAGQGGGSGLNDIFEAQKIEWHECHHHHHH

SEQ ID NO: 28 - protein, 157 aa, synthetic construct
MAQAAAGDEAQRWENQGAAYAARGDYDRAIEYYERALELDPNNAWAWWELGVAYAARGDYDRAIEYYERALELDPDDATAWSRLGHAYAARGDYDRAIEYYERALELDPEDETARYLLEHARRARQGQAGQGGGSGLNDIFEAQKIEWHECHHHHHH

SEQ ID NO: 29 - protein, 157 aa, synthetic construct
MAQAAAGDEAARWEWQGAAYAARGDYDRAIEYYERALELDPNNAWAWWELGVAYAARGELDRAIEYYERALELDPDDAHAWFRLGWAYAARGDYDRAIEYYERALELDPEDELAREELEEARRARQGQAGQGGGSGLNDIFEAQKIEWHECHHHHHH

SEQ ID NO: 30 - protein, 157 aa, synthetic construct
MAQAAAGDEAQRWENQGAAYAARGDYDRAIEYYERALELDPNNAWAWWELGVAYAARGDYDRAIEYYERALELDPDDAAAWRRLGHAYAARGDYDRAIEYYERALELDPEDETARYLLEHARRARQGQAGQGGGSGLNDIFEAQKIEWHECHHHHHH

SEQ ID NO: 31 - protein, 157 aa, synthetic construct
MAQAAAGDEAQRWENQGAAYAARGDYDRAIEYYERALELDPNNAWAWWDLGVAYAARGDYDRAIEYYERALELDPDDAAAWRRLGHAYAARGDYDRAIEYYERALELDPEDELARRILEDARRARQGQAGQGGGSGLNDIFEAQKIEWHECHHHHHH

SEQ ID NO: 32 - protein, 157 aa, synthetic construct
MAQAAAGDEAGRWEGQGAAYAARGDYDRAIEYYERALELDPNNAWAWWELGVAYAARGDYDRAIEYYERALELDPDDAAAWRRLGHAYAARGDYDRAIEYYERALELDPEDELARRILEDARRARQGQAGQGGGSGLNDIFEAQKIEWHECHHHHHH

SEQ ID NO: 33 - protein, 157 aa, synthetic construct
MAQAAAGDEAGRWEGQGAAYAARGDYDRAIEYYERALELDPNNAWAWWELGVAYAARGELDRAIEYYERALELDPDDAHAWFRLGWAYAARGDYDRAIEYYQRALELDPNNEVARSQLEHARRARRGQAGQGGGSGLNDIFEAQKIEWHECHHHHHH

SEQ ID NO: 34 - protein, 157 aa, synthetic construct
MAQAAAGDEAQRWENQGAAYAARGDYDRAIEYYERALELDPNNAWAWWELGVAYAARGELDRAIEYYERALELDPDDAHAWFRLGWAYAARGDYDRAIEYYERALELDPEDELAREELEEARRARRGQAGQGGGSGLNDIFEAQKIEWHECHHHHHH

SEQ ID NO: 35 - protein, 157 aa, synthetic construct
MAQAAAGDEAGRWEGQGAAYAARGDYDRAIEYYERALELDPNNAWAWWDLGVAYAARGDYDRAIEYYERALELDPDDATAWSRLGHAYAARGDYDRAIEYYERALELDPEDELARQQLEWARRARQGQAGQGGGSGLNDIFEAQKIEWHECHHHHHH

SEQ ID NO: 36 - protein, 157 aa, synthetic construct
MAQAAAGDEAQRWETLGAAYAARGDYDRAIEYYQRALELDPNNAWAWWELGVAYAARGDYDRAIEYYERALELDPDDATAWSRLGHAYAARGDYDRAIEYYQRALELDPNNEVARSQLEHARRARRGQAGQGGGSGLNDIFEAQKIEWHECHHHHHH

SEQ ID NO: 37 - protein, 157 aa, synthetic construct
MAQAAAGDEALRWWDQGAAYAARGDYDRAIEYYERALELDPNNAWAWWDLGVAYAARGDYDRAIEYYERALELDPDDAHAWFRLGWAYAARGDYDRAIEYYERALELDPEDELAREELEEARRARQGQAGQGGGSGLNDIFEAQKIEWHECHHHHHH

SEQ ID NO: 38 - protein, 157 aa, synthetic construct
MAQAAAGDEAGRWEGQGAAYAARGDYDRAIEYYERALELDPNNAWAWWELGTAYAARGDYDRAIEYYERALELDPDDAIAWGRLGHAYAARGDYDRAIEYYERALELDPEDELARQQLEWARRARQGQAGQGGGSGLNDIFEAQKIEWHECHHHHHH

SEQ ID NO: 39 - protein, 157 aa, synthetic construct
MAQAAAGDEAGRWEGQGAAYAARGDYDRAIEYYERALELDPNNAWAWWDLGVAYAARGDYDRAIEYYERALELDPDDATAWNRLGHAYAARGDYDRAIEYYQRALELDPNNEVARSQLEHARRARRGQAGQGGGSGLNDIFEAQKIEWHECHHHHHH

SEQ ID NO: 40 - protein, 157 aa, synthetic construct
MAQAAAGDEAQRWENQGAAYAARGDYDRAIEYYERALELDPNNAWAWWDLGVAYAARGDYDRAIEYYERALELDPDDATAWSRLGHAYAARGDYDRAIEYYERALELDPEDETARYLLEHARRARQGQAGQGGGSGLNDIFEAQKIEWHECHHHHHH

SEQ ID NO: 41 - protein, 157 aa, synthetic construct
MAQAAAGDEAQRWETLGAAYAARGDYDRAIEYYQRALELDPNNAWAWWELGVAYAARGDYDRAIEYYQRALELDPDDAAAWRRLGHAYAARGDYDRAIEYYERALELDPEDELARRILEDARRARQGQAGQGGGSGLNDIFEAQKIEWHECHHHHHH

SEQ ID NO: 42 - protein, 157 aa, synthetic construct
MAQAAAGDEAGRWEGQGAAYAARGDYDRAIEYYERALELDPNNAWAWWDLGVAYAARGDYDRAIEYYERALELDPDDAAAWNRLGHAYAARGDYDRAIEYYQRALELDPNNEVARSQLEHARRARRGQAGQGGGSGLNDIFEAQKIEWHECHHHHHH

SEQ ID NO: 43 - protein, 157 aa, synthetic construct
MAQAAAGDEAQRWENQGAAYAARGDYDRAIEYYERALELDPNNAWAWWELGVAYAARGDYDRAIEYYERALELDPDDAAAWRRLGHAYAARGDYDRAIEYYERALELDPEDELAREELEEARRARQGQAGQGGGSGLNDIFEAQKIEWHECHHHHHH

SEQ ID NO: 44 - protein, 157 aa, synthetic construct
MAQAAAGDEAQRWENQGAAYAARGDYDRAIEYYERALELDPNNAWAWWELGVAYAARGDYDRAIEYYERALELDPDDATAWNRLGHAYAARGDYDRAIEYYERALELDPEDETARYLLEHARRARQGQAGQGGGSGLNDIFEAQKIEWHECHHHHHH

SEQ ID NO: 45 - protein, 157 aa, synthetic construct
MAQAAAGDEAGRWEGQGAAYAARGDYDRAIEYYERALELDPNNAWAWWDLGVAYAARGDYDRAIEYYERALELDPDDAAAWRRLGHAYAARGDYDRAIEYYERALELDPEDELARRILEDARRARQGQAGQGGGSGLNDIFEAQKIEWHECHHHHHH

SEQ ID NO: 46 - protein, 157 aa, synthetic construct
MAQAAAGDEAARWEWQGAAYAARGDYDRAIEYYERALELDPNNAWAWWELGNAYAARGDYDRAIEYYERALELDPDDATAWSRLGHAYAARGDYDRAIEYYERALELDPEDETARYLLEHARRARRGQAGQGGGSGLNDIFEAQKIEWHECHHHHHH

SEQ ID NO: 47 - protein, 157 aa, synthetic construct
MAQAAAGDEAARWEWQGAAYAARGDYDRAIEYYERALELDPNNAWAWWELGVAYAARGELDRAIEYYERALELDPDDAHAWFRLGWAYAARGDYDRAIEYYQRALELDPEDELARRILEDARRARQGQAGQGGGSGLNDIFEAQKIEWHECHHHHHH

SEQ ID NO: 48 - protein, 157 aa, synthetic construct
MAQAAAGDEAVRWEIQGAAYAARGDYDRAIEYYERALELDPNNAWAWWELGVAYAARGDYDRAIEYYERALELDPDDAHAWFRLGWAYAARGDYDRAIEYYERALELDPEDELARRILEDARRARQGQAGQGGGSGLNDIFEAQKIEWHECHHHHHH

SEQ ID NO: 49 - protein, 157 aa, synthetic construct
MAQAAAGDEAQRWETLGAAYAARGDYDRAIEYYQRALELDPNNAWAWWELGVAYAARGDYDRAIEYYQRALELDPNNATAWNRLGHAYAARGDYDRAIEYYQRALELDPNNEVARSQLEHARRARRGQAGQGGGSGLNDIFEAQKIEWHECHHHHHH

SEQ ID NO: 50 - protein, 157 aa, synthetic construct
MAQAAAGDEAGRWEGQGAAYAARGDYDRAIEYYERALELDPNNAWAWWDLGVAYAARGDYDRAIEYYERALELDPDDAAAWRRLGHAYAARGDYDRAIEYYERALELDPEDETARYLLEHARRARQGQAGQGGGSGLNDIFEAQKIEWHECHHHHHH

SEQ ID NO: 51 - protein, 157 aa, synthetic construct
MAQAAAGDEAARWEWQGAAYAARGDYDRAIEYYERALELDPNNAWAWWELGVAYAARGELDRAIEYYERALELDPDDAHAWFRLGWAYAARGDYDRAIEYYERALELDPEDELARRILEDARRARQGQAGQGGGSGLNDIFEAQKIEWHECHHHHHH

SEQ ID NO: 52 - protein, 157 aa, synthetic construct
MAQAAAGDEAQRWENQGAAYAARGDYDRAIEYYERALELDPNNAWAWWELGVAYAARGDYDRAIEYYERALELDPDDAAAWRRLGHAYAARGDYDRAIEYYERALELDPEDELARRILEDARRARQGQAGQGGGSGLNDIFEAQKIEWHECHHHHHH

SEQ ID NO: 53 - protein, 157 aa, synthetic construct
MAQAAAGDEAQRWENQGAAYAARGDYDRAIEYYERALELDPNNAWAWWELGVAYAARGELDRAIEYYERALELDPDDAHAWFRLGWAYAARGDYDRAIEYYERALELDPEDELARRILEDARRARQGQAGQGGGSGLNDIFEAQKIEWHECHHHHHH

SEQ ID NO: 54 - protein, 157 aa, synthetic construct
MAQAAAGDEAQRWETLGAAYAARGDYDRAIEYYQRALELDPNNAWAWWELGVAYAARGDYDRAIEYYERALELDPDDAAAWRRLGHAYAARGDYDRAIEYYERALELDPEDELARRILEDARRARQGQAGQGGGSGLNDIFEAQKIEWHECHHHHHH

SEQ ID NO: 55 - protein, 157 aa, synthetic construct
MAQAAAGDEAQRWEHLGAAYAARGDYDRAIEYYQRALELDPNNAWAWWELGVAYAARGDYDRAIEYYQRALELDPNNAVAWNRLGHAYAARGDYDRAIEYYQRALELDPNNEFARQSLEEARRARRGQAGQGGGSGLNDIFEAQKIEWHECHHHHHH

SEQ ID NO: 56 - protein, 157 aa, synthetic construct
MAQAAAGDEANRWYVLGAAYAARGDYDRAIEYYQRALELDPNNAYAWDELGSAYAARGDYDRAIEYYQRALELDPNNAVAWSRLGQAYAARGDYDRAIEYYQRALELDPNNETARHLLEWARRARRGQAGQGGGSGLNDIFEAQKIEWHECHHHHHH

SEQ ID NO: 57 - protein, 157 aa, synthetic construct
MAQAAAGDEAERWYDLGAAYAARGDYDRAIEYYQRALELDPNNAWAWWELGVAYAARGDYDRAIEYYQRALELDPNNAVAWARLGIAYAARGDYDRAIEYYQRALELDPNNEVARDALEYARRARRGQAGQGGGSGLNDIFEAQKIEWHECHHHHHH

SEQ ID NO: 58 - protein, 157 aa, synthetic construct
MAQAAAGDEAERWESQGAAYAARGDYDRAIEYYERALELDPNNAWAWWELGTAYAARGDYDRAIEYYERALELDPDDARAWSRLGHAYAARGDYDRAIEYYERALELDPEDETARANLEDARRARRGQAGQGGGSGLNDIFEAQKIEWHECHHHHHH

SEQ ID NO: 59 - protein, 157 aa, synthetic construct
MAQAAAGDEAQRWEEQGAAYAARGDYDRAIEYYERALELDPNNAWAWWELGTAYAARGDYDRAIEYYERALELDPDDARAWSRLGHAYAARGDYDRAIEYYERALELDPEDETARFLLEQARRARRGQAGQGGGSGLNDIFEAQKIEWHECHHHHHH

SEQ ID NO: 60 - protein, 160 aa, synthetic construct
MGGGGSAAAGDEAIRWNTLGYAYGWRGDYDRAIEYYQRALELDPNNATAWAALGYAYQERGDYDRAIEYYQRALELDPNNAHAWQYLGFAYWNRGDYDRAIEYYQRALELDPNNESARSYLEDARRTRRGQAGQGGGSGLNDIFEAQKIEWHECHHHHHH

SEQ ID NO: 61 - protein, 160 aa, synthetic construct
MGGGGSAAAGDEADRWYRLGSAYWDRGDYDRAIEYYQRALELDPNNAGAWYWLGSAYWHRGDYDRAIEYYQRALELDPNNAYAWSELGHAYIDRGDYDRAIEYYQRALELDPNNEFARGQLEGARRLRRGQAGQGGGSGLNDIFEAQKIEWHECHHHHHH

SEQ ID NO: 62 - protein, 160 aa, synthetic construct
MGGGGSAAAGDEADRWSTQGAAYAARGDYDRAIEYYQRALELDPNNAWAWWDLGVAYAARGDYDRAIEYYQRALELDPNNAVAWARLGIAYAARGDYDRAIEYYQRALELDPNNEVARDALEYARRARRGQAGQGGGSGLNDIFEAQKIEWHECHHHHHH

SEQ ID NO: 63 - protein, 160 aa, synthetic construct
MGGGGSAAAGDEAGRWEGQGAAYAARGDYDRAIEYYERALELDPNNAWAWWDLGVAYAARGDYDRAIEYYQRALELDPNDATAWNRLGHAYAARGDYDRAIEYYQRALELDPNNEVARQLLEYARRARRGQAGQGGGSGLNDIFEAQKIEWHECHHHHHH

SEQ ID NO: 64 - protein, 160 aa, synthetic construct
MGGGGSAAAGDEAVRWEELGAAYAARGDYDRAIEYYQRALKLDPNNAWAWWELGVAYAARGDYDRAIEYYQRALELDPNNAVAWARLGWAYAARGDYDRAIEYYQRALELDPNNESARTGLKLARRARRGQAGQGGGSGLNDIFEAQKIEWHECHHHHHH

SEQ ID NO: 65 - protein, 160 aa, synthetic construct
MGGGGSAAAGDEAFRWWDQGAAYTARGDYDRAIEYYQRALELDPNNAWAWWELGVAYAARGDYDRAIEYYQRALELDPNNAVAWNRLGHAYAARGDYDRAIEYYQRALELDPNNEVARDQLERARRARRGQAGQGGGSGLNDIFEAQKIEWHECHHHHHH

SEQ ID NO: 66 - protein, 160 aa, synthetic construct
MGGGGSAAAGDEAARWEWQGAAYAARGDYDHAIEYYERALELDPNNAWAWWELGVAYAARGELDRAIEYYERALELDPDDAHAWFRLGWAYAARGDYDRAIEYYERALELDPEDELAREELEEARRARQGQAGQGGGSGLNDIFEAQKIEWHECHHHHHH

SEQ ID NO: 67 - protein, 160 aa, synthetic construct
MGGGGSAAAGDEADRWADLGAAYAARGDYDRAIEYYQRALELDPNNAWAWWDLGVAYAARGDYDRAIEYYQRALELDPNNAVAWRRLGHAYAARGDYDRAIEYYQRALELDPNNELAREQLERARRARRGQAGQGGGSGLNDIFEAQKIEWHECHHHHHH

SEQ ID NO: 68 - protein, 160 aa, synthetic construct
MGGGGSAAAGDEADRWADLGAAYAARGDYDRAIEYYQRALELDPNNAWAWWDLGVAYAARGDYDRAIEYYQRALELDPNNAVAWRRLGHAYAARGDYDRATEYYQRALELDPNNELAREQLEHARRARRGQAGQGGGSGLNDIFEAQKIEWHECHHHHHH

SEQ ID NO: 69 - protein, 160 aa, synthetic construct
MGGGGSAAAGDEAERWYDLGAAYAARGDYDRAIEYYQRALELDPNNAWAWWELGVAYAARGDYDRAIEYYQRALELDPNNAVAWSRLGQAYAARGDYDRAIEYYQRALELDPNNETARHLLEWARRARRGQAGQGGGSGLNDIFEAQKIEWHECHHHHHH

SEQ ID NO: 70 - protein, 160 aa, synthetic construct
MGGGGSAAAGDEAARWEVQGAAYAARGDYDRAIEYYERALELDPNNAWAWEHLGWAYAARGDYDRAIEYYERALELDPDDAHAWFRLGFAYAARGDYDRAIEYYERALELDPEDELARAELESARRARQGQAGQGGGSGLNDIFEAQKIEWHECHHHHHH

SEQ ID NO: 71 - protein, 160 aa, synthetic construct
MGGGGSAAAGDEANRWYYLGAAYAARGDYDRAIEYYQRALELDPNNAFAWDELGNAYAARGDYDRAIEYYQRALELDPNNAVAWNRLGHAYAARGDYDRAIEYYQRALELDPNNEVARTLLEQARRARRGQAGQGGGSGLNDIFEAQKIEWHECHHHHHH

SEQ ID NO: 72 - protein, 160 aa, synthetic construct
MGGGGSAAAGDEAWRWYSQGAAYAARGDYDRAIEYYERALELDPNNAWAWWELGTAYAARGDYDRAIEYYERALELDPDDAIAWGRLGHAYAARGDYDRAIEYYERALELDPEDELARQQLEWARRARQGQAGQGGGSGLNDIFEAQKIEWHECHHHHHH

SEQ ID NO: 73 - protein, 160 aa, synthetic construct
MGGGGSAAAGDEADRWWALGAAYAARGDYDRAIEYYQRALELDPNNAWAWWDLGVAYAARGDYDRAVEYYQRALELDPNNAVAWRRLGHAYAARGDYDRAIEYYQRALELDPNNELAREQLERARRARHGQAGQGGGSGLNDIFEAQKIEWHECHHHHHH

SEQ ID NO: 74 - protein, 160 aa, synthetic construct
MGGGGSAAAGDEANRWYYLGAAYAARGDYDRAIEYYQRALELDPNNAFAWDELGNAYAAREDYDRAIEYYQRALELDPNNAVAWNRLGHAYAARGDYDRAIEYYQRALELDPNNEVARTLLEQARRARRGQAGQGGGSGLNDIFEAQKIEWHECHHHHHH

SEQ ID NO: 75 - protein, 160 aa, synthetic construct
MGGGGSAAAGDEAERWYDLGAAYAARGDYDRAIEYYQRALELDPNNAWAWWELGVAYAARGDYDRAIEYYQRALELDPNNAVAWARLGIAYAARGDYDRAIEYYQRALELDPNNEVARTLLEQARRARRGQAGQGGGSGLNDIFEAQKIEWHECHHHHHH

SEQ ID NO: 76 - protein, 160 aa, synthetic construct
MGGGGSAAAGDEAGRWEGQGAAYAARGDYDRAIEYYERALELDPNNAWAWWDLGVAYAARGDYDRAIEYYERALELDPDDATAWSRLGHAYAARGDYDRAIEYYERALQLDPEDETARYLLEHARRARQGQAGQGGGSGLNDIFEAQKIEWHECHHHHHH

SEQ ID NO: 77 - protein, 160 aa, synthetic construct
MGGGGSAAAGDEADRWYELGAGYAARGDYDRAIEYYQRALELDPNNAWAWWELGVAYAARGDYDRAIEYYQRALELDPNNATAWQRLGHAYAARGDYDRAIEYYQRALELDPNNEAARYQLEFARRARRGQAGQGGGSGLNDIFEAQKIEWHECHHHHHH

SEQ ID NO: 78 - protein, 160 aa, synthetic construct
MGGGGSAAAGDEASRWEIQGAAYAARGDYDRAIEYYERALELDPNNAWAWWDLGVAYAARGDYDRAIEYYQRALELDPNNAVAWRRLGHAYAARGDYDRAIEYYQRALELDPNNELAREQLERARRARRGQAGQGGGSGLNDIFEAQKIEWHECHHHHHH

SEQ ID NO: 79 - protein, 160 aa, synthetic construct
MGGGGSAAAGDEADRWADLGAAYAARGDYDRAIEYYQRALELDPNNAWAWWDLGVAYAARGDYDRAIEYYQRALELDPNNAVAWRRLGHAYAARGDYDRAIEYYQRALELDPNNELAREQLERARRARHGQAGQGGGSGLNDIFEAQKIEWHECHHHHHH

SEQ ID NO: 80 - protein, 160 aa, synthetic construct
MGGGGSAAAGDEADRWAALGAAYAARGDYDRAIEYYQRALELDPNNAWAWWDLGVAYAARGDYDRAIEYYERALELDPDDAHAWFRLGWAYAARGDYDRAIEYYERALELDPEDETARDVLEGARRARQGQAGQGGGSGLNDIFEAQKIEWHECHHHHHH

SEQ ID NO: 81 - protein, 160 aa, synthetic construct
MGGGGSAAAGDEAERWYDLGAAYAARGDYDRAIEYYQRALELDPNNAWAWWELGVAYAARGDYDRAIEYYQRALELDPNNAVAWRRLGIAYAARGDYDRAIEYYQRALELDPNNEVARDALEYARRARRGQAGQGGGSGLNDIFEAQKIEWHECHHHHHH

SEQ ID NO: 82 - protein, 160 aa, synthetic construct
MGGGGSAAAGDEAGRWYDLGAAYAARGDYDRAIEYYQRALELDPNNAWAWWELGVAYAARGDYDRAIEYYQRALELDPNNAVAWARLGIAYAARGDYDRAIEYYQRALELDPNNEVARDALEYARRARRGQAGQGGGSGLNDIFEAQKIEWHECHHHHHH

SEQ ID NO: 83 - protein, 160 aa, synthetic construct
MGGGGSAAAGDEADRWADLGAAYAARGDYDRAIEYYQRALELDPNNAWAWWDLGVAYAARGDYDRAIEYYQRALELDPNNAVAWRRLGHAYAARGDYDRAIEYYQRALELDPNNELAREQLGRARRARRGQAGQGGGSGLNDIFEAQKIEWHECHHHHHH

SEQ ID NO: 84 - protein, 160 aa, synthetic construct
MGGGGSAAAGDEGGRWEGQGAAYAARGDYDRAIEYYERALELDPNNAWAWWDLGVAYAARGDYDRAIEYYERALELDPDDATAWSRLGHAYAARGDYDRAIEYYERALELDPEDETARYLLEHARRARQGQAGQGGGSGLNDIFEAQKIEWHECHHHHHH

SEQ ID NO: 85 - protein, 160 aa, synthetic construct
MGGGGSAAAGDEAERWDYLGAAYAARGDYDRAIEYYQRALELDPNNAWAWWDLGLAYAARGDYDRAIEYYQRALELDPNNAVAWFRLGWAYAARGDYDRAIEYYQRALELDPNNELARSELENARRARRGQAGQGGGSGLNDIFEAQKIEWHECHHHHHH

SEQ ID NO: 86 - protein, 160 aa, synthetic construct
MGGGGSAAAGDEAQRWENQGAAYAARGDYDRAIECYESALELDPNNAWAWWELGVAYAARGDYDRAIEYYERALELDPDDAIAWSRLGHAYAARGDYDRAIEYYERALELDPEDETARYLLEHARRARQGQAGQGGGSGLNDIFEAQKIEWHECHHHHHH

SEQ ID NO: 87 - protein, 160 aa, synthetic construct
MGGGGSAAAGDEAQHWENQGAAYAARGDYDRAIEYYERALELDPNNAWAWWELGVAYAARGDYDRAIEYYERALELDPDDATVWSRLGHAYAARGDYDRAIEYYERALELDPEDETARYLLEHARRARQGQAGQGGGSGLNDIFEAQKIEWHECHHHHHH

SEQ ID NO: 88 - protein, 160 aa, synthetic construct
MGGGGSAAAGDEAQCWENQGAAYAARGDYDRAIEYYERALELDPNNAWAWWELGVAYAARGDYDRAIEYYERALELDPDDATAWSRLGHAYAARGDYDRAIEYYERALELDPEDETARYLLEHARRARQGQAGQGGGSGLNDIFEAQKIEWHECHHHHHH

SEQ ID NO: 89 - protein, 160 aa, synthetic construct
MGGGGSAAAGDEAQHWENQGAAYAARGDYDRAIEYYGRALELDPNNAWAWWELGVAYAARGDYDRAIEYYERALELDPDDATTWSRLGHAYAARGDYDRAIEYYERALELDPEDETARYLLEHARRARQGQAGQGGGSGLNDIFEAQKIEWHECHHHHHH

SEQ ID NO: 90 - protein, 160 aa, synthetic construct
MGGGGSAAAGDEAQCWENQGAAYAARGDYDRAIEYYERALELDPNNAWAWWELGVAYAARGDYDRAIEYYERALELDPDDATTWSRLGHAYAARGDYDRAIEYYVRALELDPEDETARYLLEHARRARQGQAGQGGGSGLNDIFEAQKIEWHECHHHHHH

SEQ ID NO: 91 - protein, 160 aa, synthetic construct
MGGGGSAAAGDEAQRWENQGAAYAARGDYDRAIEYYGRALELDPNNAWAWWELGVAYAARGDYDRAIEYYERALELDPDDATAWSRLGHAYAARGDYDRAIEYYERALELDPENETARYLLEHARRARQGQAGQGGGSGLNDIFEAQKIEWHECHHHHHH

SEQ ID NO: 92 - protein, 160 aa, synthetic construct
MGGGGSAAAGDEAQRWENQGAAYAARGDYDRAIEYYERALELEPNNAWAWWELGVAYAARGDYDRAIEYYERALELDPDDATAWSRLGHAYAARGDYDRAIGYYERALELDPENETARYLLEHARRARQGQAGQGGGSGLNDIFEAQKIEWHECHHHHHH

SEQ ID NO: 93 - protein, 160 aa, synthetic construct
MGGGGSAAAGDEAQRWENQGAAYAARGDYDRAIEYYERALELDPNNAWAWWELGVAYAARGDFDRAIEYYERALELDPDDATVWSRLGHAYAARGDYDRAIEYYERALELDPVDETARYLLEHARRARQGQAGQGGGSGLNDIFEAQKIEWHECHHHHHH

SEQ ID NO: 94 - protein, 160 aa, synthetic construct
MGGGGSAAAGDEAVRWEIQGAAYAARGDYDRAIEYYERALELDPNNAWAWWELGVAYAARGDYDRAIEYYERALELDPDDAHVWFRLGWAYAVRGDYDRAIEYYERALELDPEDELARRILEDARRARQGQAGQGGGSGLNDIFEAQKIEWHECHHHHHH

SEQ ID NO: 95 - protein, 160 aa, synthetic construct
MGGGGSAAAGDEAVRWEIQGAAYAARGDYNRAIEYYERALELDPNNAWAWWELGVAYAARGDYDRAIEYYERALNLDPDDAHTWFRLGWAYAARGDYARAIEYYERALELDPEDELVRRILEDARRARQGQAGQGGGSGLNDIFEAQKIEWHECHHHHHH

SEQ ID NO: 96 - protein, 160 aa, synthetic construct
MGGGGSAAAGDEAVRWEIQGAAYAARGDYDLAIEYYERTLELDPDNAWAWWELGVAYAARGDYDRAIEYYERALELDPDDAHVWFRLGWAYATRGDYDRAIEYYERALELDPEDELARRILEDARRARQGQAGQGGGSGLNDIFEAQKIEWHECHHHHHH

SEQ ID NO: 97 - protein, 160 aa, synthetic construct
MGGGGSAAAGDEAARWEIQGAAYAARGDYDRAIEYYERALELDPNNAWAWWELGVAYAARGDYDRAIEYYERALELDPDDAHTWFRLGWAYAARGDYDRAIAYYERALELDPEDELTRRILGDARRVRQGQAGQGGGSGLNDIFEAQKIEWHECHHHHHH

SEQ ID NO: 98 - protein, 160 aa, synthetic construct
MGGGGSAAAGDEAVRWEIQGAAYAARGDYDRAIEYYERALELDPNNAWAWWELGVAYAARGDYDRAIEYYERALELDPDDARVWFRLGWAYAARGDYDRAIEYYERALELDPEDELARRILEDTRRARQGQAGQGGGSGLNDIFEAQKIEWHECHHHHHH

SEQ ID NO: 99 - protein, 160 aa, synthetic construct
MGGGGSAAAGDEVVHWEIQGAAYAARGDYDRAIEYYERALELDPNNAWAWWELGVAYAARGDYDRAIEYYERALELDPDDAHVWFRLGWAYAARGDYDRAIEYYERALELDPEDELARRILEDARRARQGQAGQGGGSGLNDIFEAQKIEWHECHHHHHH

SEQ ID NO: 100 - protein, 160 aa, synthetic construct
MGGGGSAAAGDEAVRWEIQGAAYAARGDYDRAIEYYERALELDPNNAWAWWELGVAYAARGDYDRAIEYYERALELDPDDAHVWFRLGWAYATRGDYDRAIEYYERALELDPEDELARRILEDARRARQGQAGQGGGSGLNDIFEAQKIEWHECHHHHHH

SEQ ID NO: 101 - protein, 160 aa, synthetic construct
MGGGGSAAAGDEALRWEIQGAAYAARGDYDRAIEYYERALELDPNNAWAWWELGVAYAARGDYDRAIEYYERALELDPDDAHVWFRLGWAYAARGDHDRAIEYYERALELDPEDELARRILEDARRARQGQAGQGGGSGLNDIFEAQKIEWHECHHHHHH

SEQ ID NO: 102 - protein, 160 aa, synthetic construct
MGGGGSAAAGDEAERWYDLGAAYAARGDYDRAIEYYQRALELDPNDAWAWWELGVAYAARGDYDRAIEYYQRALELDLNNAVAWARLGIAYAARGDYDRAIEYYQRALELDPNNEVARDALEYARRARRGQAGQGGGSGLNDIFEAQKIEWHECHHHHHH

SEQ ID NO: 103 - protein, 160 aa, synthetic construct
MGGGGSAAAGDEAERWYDLGAAYAARGDYDRAFEYYQRALELDPNNAWAWWELGVANAARGDYDRAIENYQRALELDPNNAVAWARLGVAYAARGDYDRAIEYYQRALELDPNQVIARGALEYARRARRGQAGQGGGSGLNDIFEAQKIEWHECHHHHHH

SEQ ID NO: 104 - protein, 160 aa, synthetic construct
MGGGGSAAAGDEAERWYDLGAAYAARGDYDRAIEYYQRVLELDPNNAWAWWELGVGYAARGDYDRAIEYYQRALELDPNNTVAWARLGIAYAARGDYDRAIEYYQRALELDPNNEVARDALEYARRARRGQAGQGGGSGLNDIFEAQKIEWHECHHHHHH

SEQ ID NO: 105 - protein, 160 aa, synthetic construct
MGGGGSAAAGDEAERWYDLGAAYAARGDFDRAIEYYRRTLELDPNDAWAWWELGVAYAARGDYDRAIEYYQRALELDPNNAVAWARLGIAYATRGDYDRAIEYYQRALELDPNNEVARDALEYARRVRRGQAGQGGGSGLNDIFEAQKIEWHECHHHHHH

SEQ ID NO: 106 - protein, 160 aa, synthetic construct
MGGGGSAAAGDEAERWYDLGAAYAARGDYDRATEFYQRALELDPNDAWAWWELGIAYAARGDYDRAIEYYQRALELDPNNAVAWARLGIAYAARGDYDRAIEYYQRALELDPNNEVARDALEYARRARRGQAGQGGGSGLNDIFEAQKIEWHECHHHHHH

SEQ ID NO: 107 - protein, 160 aa, synthetic construct
MGGGGSAAAGDEAERWYDLGAAYAARGDYDRAIEYYQRALELDPNDAWAWWELGVAYAARGDYDRAIEYYQRALELDPNNAVAWARLGIAYAARGDYDRAIEYYQRALELDPNNEVARDALEYARRARRGQAGQGGGSGLNDIFEAQKIEWHECHHHHHH

SEQ ID NO: 108 - protein, 160 aa, synthetic construct
MGGGGSAAAGDEAERWYDLGAAYAARGDYDRAIEYYQRALEHDPNDAWAWWELGVAYAARGDYDRAIEYYQRALELDTNNAVAWARLGIAYAARGDYDRAIEYYQRALELDPNNEVARDALEYARRARRGQAGQGGGSGLNDIFEAQKIEWHECHHHHHH

SEQ ID NO: 109 - protein, 160 aa, synthetic construct
MGGGGSAAAGDEAAHWEFQGAAYAARGDYDRAIEYYERALELDPNNAWAWEHLGWAYAARGDYDRAIEYYERALELDPDDAHAWFRLGFAYAARGDYDRAIKYYERALELDPEDELARAELESARRARQGQAGQGGGSGLNDIFEAQKIEWHECHHHHHH

SEQ ID NO: 110 - protein, 160 aa, synthetic construct
MGGGGSAAAGDEAARWEVQGAAYAARGDYDRAIEYYERVLELDPNDAWAWEHLGWAYAARGDYDRAIEYYERALEVDPDDAHAWFRLGFAYAARGDYDRAMEYYERALELDPEDELARAELESARRARQGQAGQGGGSGLNDIFEAQKIEWHECHHHHHH

SEQ ID NO: 111 - protein, 160 aa, synthetic construct
MGGGGSAAAGDEAARWEVQGAAYAARGDYDRAIEYYERALELDPNNAWAWEHLGWAYAARGDYDRAIEYYERALELDPDDAHAWFRLGFAYAARGDYDRAIEYYERALELDPNNELAREQLEHARRARRGQAGQGGGSGLNDIFEAQKIEWHECHHHHHH

SEQ ID NO: 112 - protein, 160 aa, synthetic construct
MGGGGSAAAGDEAAHWEVQGAAYAARGDYDRAIEYYERALELDPNNAWAWEHLGWAYAARGDFDRAIEYYERALELDPDDAHAWFRLGFAYAARGDYDRAIEYYERALELDPEDELARAELESARRARQGQAGQGGGSGLNDIFEAQKIEWHECHHHHHH

SEQ ID NO: 113 - protein, 160 aa, synthetic construct
MGGGGSAAAGDEAARWEVLGAAYAARGDYDRAIEYYERALELDPNNAWAWEHLGWAYAARGDYDRAIEYYERALGLDPDDAHAWFRLGFAYAARGDYDRAIEYYERALELDPENELARAELESARRARQGQAGQGGGSGLNDIFEAQKIEWHECHHHHHH

SEQ ID NO: 114 - protein, 160 aa, synthetic construct
MGGGGSAAAGDEAARWEVQGAAYAARGDYDRAIEYYERALELDPNNAWAWEHLGWAYAARGDYDRAIEYYERALELDPDDAHSWFRLGFAYAVRGDYDRAIEYYERALELDPEDELARAELESARRARQGQAGQGGGSGLNDIFEAQKIEWHECHHHHHH

SEQ ID NO: 115 - protein, 160 aa, synthetic construct
MGGGGSAAAGDEVARWEVQGAAYAARGGYDRAIEYYERALELDPNNAWAWEHLGWAYAARGDYDRAIEYYERALELDPDDAHEWFRLGFAYAARGDYDRAIEYYERALELDPEDELARAELESARRARQGQAGQGGGSGLNDIFEAQKIEWHECHHHHHH

SEQ ID NO: 116 - protein, 160 aa, synthetic construct
MGGGGSAAAGDEAARWEVQGAAYAARGDYYRAIEYYERALELDPNNAWAWEHLGWAYAARGDYDRAFEYYERALELDPDDAHVWFRLGFAYAVRGDYDRAIEYYERALELDPEDELARAELESARRARQGQAGQGGGSGLNDIFEAQKIEWHECHHHHHH

SEQ ID NO: 117 - protein, 160 aa, synthetic construct
MGGGGSAAAGDEAARWEVQGAAYAARGDYDRAIEYYERALELDPSNAWAWEHLAWAYAARGDYDRAIECYERALELDPDDAHAWFRLGFSYAARGDYDRAIEYYERALELDPEDELARAELESARRARQGQAGQGGGSGLNDIFEAQKIEWHECHHHHHH

SEQ ID NO: 118 - protein, 160 aa, synthetic construct
MGGGGSAAAGDEVARWEVQGAAYAARGDYDRAIEYYERALELDPNNAWAWEHLGWAYAARGDYDRAIEYYERALELDPDDAHEWFRLGFAYAARGDYDRAIEYYERALELDPEDELARAELESARRARQGQAGQGGGSGLNDIFEAQKIEWHECHHHHHH

SEQ ID NO: 119 - protein, 160 aa, synthetic construct
MGGGGSAAAGDEADRWADLGAAYAARGDYDRAIEYYQRALELDPNNAWAWWDLGVAYAARGDYDRAIEYYQRALELDPNNTVAWRRSGHAYAARGDYDRATEYYQRALELDPNNELAREQLEHARRARRGQAGQGGGSGLNDIFEAQKIEWHECHHHHHH

SEQ ID NO: 120 - protein, 160 aa, synthetic construct
MGGGGSAAAGDEADRWADLGAAYAARGDYDRAIEYYQRALEIDPNNAWAWWDLGVAYAARGDYDRAIEYYQRALELDPNNAVAWSRLGHAYAARGDYDRAIEYYQRALELDTNNEVARTLLEQARRARRGQAGQGGGSGLNDIFEAQKIEWHECHHHHHH

SEQ ID NO: 121 - protein, 160 aa, synthetic construct
MGGGGSAAAGDEANRWYYLGAACAARGDYDRAIEYYQRALELDPNNAWAWWDLGVAYAARGDYDRAIEYYQRALELDPNNAVAWRRLGHAYAARGDYDRATEYYQRALELDPNNELAREQLEHARRARRGQAGQGGGSGLNDIFEAQKIEWHECHHHHHH

SEQ ID NO: 122 - protein, 160 aa, synthetic construct
MGGGGSAAAGDEADRWADLGAAYAARGDYDRAIEYYQRALELDPNNAWAWWDLGVAYAARGDYDRAIEYYQRALELDPNNSVAWRRLGHAYAARGDCDRATEHYQRALELDPNNELAREQLEHARRARRGQAGQGGGSGLNDIFEAQKIEWHECHHHHHH

SEQ ID NO: 123 - protein, 160 aa, synthetic construct
MGGGGSAAAGDEANRWYYLGAAYAARGDYDRAIEYYQLALELDPNNAWAWWDLGVAYAARGDYDRAIEYYQRALELDPNNAVAWRRLGHAYAARGDYDRATEYYQRALELDPNNELAREQLEHARRARRGQAGQGGGSGLNDIFEAQKIEWHECHHHHHH

SEQ ID NO: 124 - protein, 160 aa, synthetic construct
MGGGGSAAAGDEANRWYYLGAAYAARGDFDRAIEYYQRALELDPNNAWAWWDLGVAYAARGDFDRAIEYYQRALELDSNNAVVWRRLGHAYAARGDYDRATEYYQRALELDPNNELAREQLEHARRARRGQAGQGGGSGLNDIFEAQKIEWHECHHHHHH

SEQ ID NO: 125 - protein, 160 aa, synthetic construct
MGGGGSAAAGDEADRWADLGAAYAARGYYDRAIEYYQRALELDPNNAWAWWDLGVAYAARGDYDRAIEYYQRALELDPNNAVVWRRLGHAYAARGDYDRATEYYQRALELDPNNELAREQLEHARRARRGQAGQGGGSGLNDIFEAQKIEWHECHHHHHH

SEQ ID NO: 126 - protein, 160 aa, synthetic construct
MGGGGSAAAGDEADRWADLGAAYAARGDYDRAIEYYQRALELDPNNAWAWWDLGVAYAARGDFDRAIEYYQRALELDSNNAVVWRRLGHAYAARGDYDRATEYYQRALELDPNNELAREQLEHARRARRGQAGQGGGSGLNDIFEAQKIEWHECHHHHHH

SEQ ID NO: 127 - protein, 160 aa, synthetic construct
MGGGGSAAAGDEADRWADLGAAYAARGDYDRAIEYYQRALELDPNNAWAWWDLGVAYAARGDYDRAIEYYQRALELDPNDAVVWRRLGHAYAARGDYDRATEYYQRALELDPNNELAREQLEHARRARRGQAGQGGGSGLNDIFEAQKIEWHECHHHHHH

SEQ ID NO: 128 - protein, 160 aa, synthetic construct
MGGGGSAAAGDEADRWADLGAAYAARGDYDRAIEYYQRALELDPNNAWAWWDLGVAYAARGDYDRAIEYYQRALELDLNNTVVWRRLGHAYAARGDYDRATEYYQRALELDPNNELAREQLEHARRARRGQAGQGGGSGLNDIFEAQKIEWHECHHHHHH

SEQ ID NO: 129 - protein, 160 aa, synthetic construct
MGGGGSAAAGDEADRWADLGAAYAARGDYDRAIEYYQRALELDPNNAWAWWDLGVAYAARGDYDRAIEYYQRALELDPNNSVAWRRSGHAYAARGDYDRATEYYQRALELDPNNELAREQLEHARRARRGQAGQGGGSGLNDIFEAQKIEWHECHHHHHH

SEQ ID NO: 130 - protein, 160 aa, synthetic construct
MGGGGSAAAGDEADRWADLGAAYAARGDYDRAIEYYQRALELDPNNAWAWWDLGVAYAARGDYDRAIEYYQRALELDPNNAVTWRRLGHAYDARGDYDRATEYYQRALELDPNNELAREQLEHARRARRGQAGQGGGSGLNDIFEAQKIEWHECHHHHHH

SEQ ID NO: 131 - protein, 160 aa, synthetic construct
MGGGGSAAAGDEADRWADLGAAYAARGDYDRAIEYYQRALELDPNNGWAWWDLGVAYAARGDYDRAIEYYQRALELDPNNAVVWRRLGHAYAARGDYDRATEYYQRALELDPNNELAREQLEHARRARRGQAGQGGGSGLNDIFEAQKIEWHECHHHHHH

SEQ ID NO: 132 - protein, 160 aa, synthetic construct
MGGGGSAAAGDEADRWADLGAAYAARGDYDRAIEYYQRALELDPNNSWAWWDLGVAYAARGDYDRAIEYYHRASELDPNNAVAWRRLGHAYAARGDYDRATEYYQRALELDPNNELAREQLEHARRARRGQAGQGGGSGLNDIFEAQKIEWHECHHHHHH

SEQ ID NO: 133 - protein, 160 aa, synthetic construct
MGGGGSAAAGDEANRWYYLGAAYAARGDYDSAIEYYQRALELDPNNAWAWWDLGVAYAARGDYDRAIEYYQRALELDPNNAVAWRRLGHAYAARGDYDRATEYYQRALELDPNNELAREQLEHARRARRGQAGQGGGSGLNDIFEAQKIEWHECHHHHHH

SEQ ID NO: 134 - protein, 203 aa, synthetic construct
MDEALRWWDQGAAYAARGDYDRAIEYYERALELDPNNAWAWWDLGVAYAARGDYDRAIEYYERALELDPDDAHAWFRLGWAYAARGDYDRAIEYYERALELDPEDELAREELEEARRARQGQAGQGWAEFRQRLAAIRTRLEALGGSEAELAAFEREIAAFESELQAYAGAGNPEVEALRREAAAIRDELQAYRHNHHHHHHC

SEQ ID NO: 135 - protein, 203 aa, synthetic construct
MDEAARWEWQGAAYAARGDYDRAIEYYERALELDPNNAWAWWELGVAYAARGELDRAIEYYERALELDPDDAHAWFRLGWAYAARGDYDRAIEYYQRALELDPEDELARRILEDARRARQGQAGQGWAEFRQRLAAIRTRLEALGGSEAELAAFEREIAAFESELQAYAGAGNPEVEALRREAAAIRDELQAYRHNHHHHHHC

SEQ ID NO: 136 - protein, 203 aa, synthetic construct
MDEAERWYDLGAAYAARGDYDRAIEYYQRALELDPNNAWAWWELGVAYAARGDYDRAIEYYQRALELDPNNAVAWARLGIAYAARGDYDRAIEYYQRALELDPNNEVARDALEYARRARRGQAGQGWAEFRQRLAAIRTRLEALGGSEAELAAFEREIAAFESELQAYAGAGNPEVEALRREAAAIRDELQAYRHNHHHHHHC

SEQ ID NO: 137 - protein, 280 aa, synthetic construct
MAQAAAGDEAARWEWQGAAYAARADYDRAIEYYERALELDPNNAWAWWELGVAYAARGELDRAIEYYERALELDPDDAHAWFRLGWAYAARGDYDRAIEYYQRALELDPEDELAREELEEARRARQGQAGQGGGSGLNDIFEAQKIEWHECHHHHHHRALELDPNNAWAWWELGVAYAARGELDRAIEYYERALELDPDDAHAWFRLGWAYAARGDYDRAIEYYQRALELDPEDELAREELEEARRARQGQAGQGGGSGLNDIFEAQKIEWHECHHHHHH

SEQ ID NO: 138 - protein, 236 aa, synthetic construct
MGGGGSAAAGDEAARWEVQGAAYAARGDYDRAIEYYERALELDPNNAWAWEHLGWAYAARGDYDRAIEYYERALELDPDDAHAWFRLGFAYAARGDYDRAIEYYERALELDPEDELARAELESARRARQGQAGQGWAEFRQRLAAIRTRLEALGGSEAELAAFEREIAAFESELQAYAGAGNPEVEALRREAAAIRDELQAYRHNGQAGQGGGSGLNDIFEAQKIEWHECHHHHHH

SEQ ID NO: 139 - protein, 160 aa, synthetic construct
MGGGGSAAAGDEAERWWRLGDAYRWRGDYDRAIEYYQRALELDPNNALAWVELGQAYTRRGDYDRAIEYYQRALGLDPNNAFAWHLLGEAYDERGDYDRAIEYYQRALELDPNNEWARELLEWARRLRRGQAGQGGGSGLNDIFEAQKIEWHECHHHHHH

SEQ ID NO: 140 - protein, 160 aa, synthetic construct
MGGGGSAEAGDEAVRWEIQGAAYAARGDYDRAIEYYERALELDPNNAWAWWELGVAYAARGDYDRAIEYYERALELDPDDAHAWFRLGWAYAARGDYDRAIEYYERALELDPEDETARDVLEGARRARQGQAGQGGGSGLNDIFEAQKIEWHECHHHHHH

SEQ ID NO: 141 - protein, 160 aa, synthetic construct
MGGGGSAAAGDEAERWRRLGHAYHVRGDYDRAIEYYQRALEPDPNNAYAWIELGHAYWRRGDYDRAIEYYQRALELDPNNAEAWVVLGIAYEQRGDYDRAIEYYQRALELDPNNEFARRHLEEARRERRGQAGQGGGSGLNDIFEAQKIEWHECHHHHHH

SEQ ID NO: 142 - protein, 203 aa, synthetic construct
MDEAVRWEIQGAAYAARGDYDRAIEYYERALELDPNNAWAWWELGVAYAARGDYDRAIEYYERALELDPDDAHVWFRLGWAYATRGDYDRAIEYYERALELDPEDELARRILEDARRARQGQAGQGWAEFRQRLAAIRTRLEALGGSEAELAAFEREIAAFESELQAYAGAGNPEVEALRREAAAIRDELQAYRHNHHHHHHC

SEQ ID NO: 143 - protein, 203 aa, synthetic construct
MDEAVRWEIQGAAYAARGDYDRAIEYYERALELDPNNAWAWWELGVAYAARGDYDRAIEYYERALELDPDDAHVWFRLGWAYAVRGDYDRAIEYYERALELDPEDELARRILEDARRARQGQAGQGWAEFRQRLAAIRTRLEALGGSEAELAAFEREIAAFESELQAYAGAGNPEVEALRREAAAIRDELQAYRHNHHHHHHC

SEQ ID NO: 144 - protein, 128 aa, synthetic construct
MGDEAERWYDLGAAYAARGDFDRAIEYYRRTLELDPNDAWAWWELGVAYAARGDYDRAIEYYQRALELDPNNAVAWARLGIAYATRGDYDRAIEYYQRALELDPNNEVARDALEYARRVRRCHHHHHH

SEQ ID NO: 145 - protein, 128 aa, synthetic construct
MGHHHHHHDEAERWYDLGAAYAARGDFDRAIEYYRRTLELDPNDAWAWWELGVAYAARGDYDRAIEYYQRALELDPNNAVAWARLGIAYATRGDYDRAIEYYQRALELDPNNEVARDALEYARRVRRC

SEQ ID NO: 146 - protein, 197 aa, synthetic construct
MAWAEFRQRLAAIRTRLEALGGSEAELAAFEREIAAFESELQAYAGAGNPEVEALRREAAAIRDELQAYRHNDEAERWYDLGAAYAARGDFDRAIEYYRRTLELDPNDAWAWWELGVAYAARGDYDRAIEYYQRALELDPNNAVAWARLGIAYATRGDYDRAIEYYQRALELDPNNEVARDALEYARRVRRHHHHHH

SEQ ID NO: 147 - protein, 141 aa, synthetic construct
MGLAEAAAREAAARAADEAERWYDLGAAYAARGDFDRAIEYYRRTLELDPNDAWAWWELGVAYAARGDYDRAIEYYQRALELDPNNAVAWARLGIAYATRGDYDRAIEYYQRALELDPNNEVARDALEYARRVRRHHHHHH

SEQ ID NO: 148 - protein, 122 aa, synthetic construct
MGDEAERWYDLGAAYAARGDFDRAIEYYRRTLELDPNDAWAWWELGVAYAARGDYDRAIEYYQRALELDPNNAVAWARLGIAYATRGDYDRAIEYYQRALELDPNNEVARDALEYARRVRRC

SEQ ID NO: 149 - protein, 128 aa, synthetic construct
MGDEAERWYDLGAAYAARGDYDRATEFYQRALELDPNDAWAWWELGIAYAARGDYDRAIEYYQRALELDPNNAVAWARLGIAYAARGDYDRAIEYYQRALELDPNNEVARDALEYARRARRCHHHHHH

SEQ ID NO: 150 - protein, 128 aa, synthetic construct
MGHHHHHHDEAERWYDLGAAYAARGDYDRATEFYQRALELDPNDAWAWWELGIAYAARGDYDRAIEYYQRALELDPNNAVAWARLGIAYAARGDYDRAIEYYQRALELDPNNEVARDALEYARRARRC

SEQ ID NO: 151 - protein, 197 aa, synthetic construct
MAWAEFRQRLAAIRTRLEALGGSEAELAAFEREIAAFESELQAYAGAGNPEVEALRREAAAIRDELQAYRHNDEAERWYDLGAAYAARGDYDRATEFYQRALELDPNDAWAWWELGIAYAARGDYDRAIEYYQRALELDPNNAVAWARLGIAYAARGDYDRAIEYYQRALELDPNNEVARDALEYARRARRHHHHHH

SEQ ID NO: 152 - protein, 141 aa, synthetic construct
MGLAEAAAREAAARAADEAERWYDLGAAYAARGDYDRATEFYQRALELDPNDAWAWWELGIAYAARGDYDRAIEYYQRALELDPNNAVAWARLGIAYAARGDYDRAIEYYQRALELDPNNEVARDALEYARRARRHHHHHH

SEQ ID NO: 153 - protein, 122 aa, synthetic construct
MGDEAERWYDLGAAYAARGDYDRATEFYQRALELDPNDAWAWWELGIAYAARGDYDRAIEYYQRALELDPNNAVAWARLGIAYAARGDYDRAIEYYQRALELDPNNEVARDALEYARRARRC

SEQ ID NO: 154 - protein, 128 aa, synthetic construct
MGDEAERWYDLGAAYAARGDYDRAIEYYQRALELDPNDAWAWWELGVAYAARGDYDRAIEYYQRALELDPNNAVAWARLGIAYAARGDYDRAIEYYQRALELDPNNEVARDALEYARRARRCHHHHHH

SEQ ID NO: 155 - protein, 128 aa, synthetic construct
MGDEAERWYDLGAAYAARGDYDRAIEYYQRALEHDPNDAWAWWELGVAYAARGDYDRAIEYYQRALELDTNNAVAWARLGIAYAARGDYDRAIEYYQRALELDPNNEVARGALEYARRARRCHHHHHH

SEQ ID NO: 156 - protein, 197 aa, synthetic construct
MAWAEFRQRLAAIRTRLEALGGSEAELAAFEREIAAFESELQAYAGAGNPEVEALRREAAAIRDELQAYRHNDEAERWYDLGAAYAARGDYDRAIEYYQRALEHDPNDAWAWWELGVAYAARGDYDRAIEYYQRALELDTNNAVAWARLGIAYAARGDYDRAIEYYQRALELDPNNEVARGALEYARRARRHHHHHH

SEQ ID NO: 157 - protein, 141 aa, synthetic construct
MGLAEAAAREAAARAADEAERWYDLGAAYAARGDYDRAIEYYQRALEHDPNDAWAWWELGVAYAARGDYDRAIEYYQRALELDTNNAVAWARLGIAYAARGDYDRAIEYYQRALELDPNNEVARGALEYARRARRHHHHHH

SEQ ID NO: 158 - protein, 128 aa, synthetic construct
MGDEAERWYDLGAAYAARGDYDRAIEYYQRALELDPNDAWAWWELGVAYAARGDYDRAIEYYQRALELDLNNAVAWARLGIAYAARGDYDRAIEYYQRALELDPNNEVARDALEYARRARRCHHHHHH

SEQ ID NO: 159 - protein, 128 aa, synthetic construct
MGHHHHHHDEAERWYDLGAAYAARGDYDRAIEYYQRALEHDPNDAWAWWELGVAYAARGDYDRAIEYYQRALELDTNNAVAWARLGIAYAARGDYDRAIEYYQRALELDPNNEVARGALEYARRARRC

SEQ ID NO: 160 - protein, 128 aa, synthetic construct
MGHHHHHHDEAERWYDLGAAYAARGDYDRAIEYYQRALELDPNDAWAWWELGVAYAARGDYDRAIEYYQRALELDLNNAVAWARLGIAYAARGDYDRAIEYYQRALELDPNNEVARDALEYARRARRC

SEQ ID NO: 161 - protein, 197 aa, synthetic construct
MAWAEFRQRLAAIRTRLEALGGSEAELAAFEREIAAFESELQAYAGAGNPEVEALRREAAAIRDELQAYRHNDEAERWYDLGAAYAARGDYDRAIEYYQRALELDPNDAWAWWELGVAYAARGDYDRAIEYYQRALELDLNNAVAWARLGIAYAARGDYDRAIEYYQRALELDPNNEVARDALEYARRARRHHHHHH

SEQ ID NO: 162 - protein, 141 aa, synthetic construct
MGLAEAAAREAAARAADEAERWYDLGAAYAARGDYDRAIEYYQRALELDPNDAWAWWELGVAYAARGDYDRAIEYYQRALELDLNNAVAWARLGIAYAARGDYDRAIEYYQRALELDPNNEVARDALEYARRARRHHHHHH

SEQ ID NO: 163 - protein, 131 aa, synthetic construct
MGHHHHHHDEAERWYDLGAAYAARGDYDRAIEYYQRALELDPNDAWAWWELGVAYAARGDYDRAIEYYQRALELDLNNAVAWARLGIAYAARGDYDRAIEYYQRALELDPNNEVARDALEYARRARRAPKC

SEQ ID NO: 164 - protein, 122 aa, synthetic construct
MGDEAERWYDLGAAYAARGDYDRAIEYYQRALELDPNDAWAWWELGVAYAARGDYDRAIEYYQRALELDLNNAVAWARLGIAYAARGDYDRAIEYYQRALELDPNNEVARDALEYARRARRC

SEQ ID NO: 165 - protein, 203 aa, synthetic construct
MDEAAHWEVQGAAYAARGDYDRAIEYYERALELDPNNAWAWEHLGWAYAARGDFDRAIEYYERALELDPDDAHAWFRLGFAYAARGDYDRAIEYYERALELDPEDELARAELESARRARQGQAGQGWAEFRQRLAAIRTRLEALGGSEAELAAFEREIAAFESELQAYAGAGNPEVEALRREAAAIRDELQAYRHNHHHHHHC

SEQ ID NO: 166 - protein, 203 aa, synthetic construct
MDEAARWEVQGAAYAARGDYDRAIEYYERVLELDPNDAWAWEHLGWAYAARGDYDRAIEYYERALEVDPDDAHAWFRLGFAYAARGDYDRAMEYYERALELDPEDELARAELESARRARQGQAGQGWAEFRQRLAAIRTRLEALGGSEAELAAFEREIAAFESELQAYAGAGNPEVEALRREAAAIRDELQAYRHNHHHHHHC

SEQ ID NO: 167 - protein, 203 aa, synthetic construct
MDEAARWEVQGAAYAARGDYDRAIEYYERALELDPNNAWAWEHLGWAYAARGDYDRAIEYYERALELDPDDAHSWFRLGFAYAVRGDYDRAIEYYERALELDPEDELARAELESARRARQGQAGQGWAEFRQRLAAIRTRLEALGGSEAELAAFEREIAAFESELQAYAGAGNPEVEALRREAAAIRDELQAYRHNHHHHHHC

SEQ ID NO: 168 - protein, 203 aa, synthetic construct
MDEVARWEVQGAAYAARGGYDRAIEYYERALELDPNNAWAWEHLGWAYAARGDYDRAIEYYERALELDPDDAHEWFRLGFAYAARGDYDRAIEYYERALELDPEDELARAELESARRARQGQAGQGWAEFRQRLAAIRTRLEALGGSEAELAAFEREIAAFESELQAYAGAGNPEVEALRREAAAIRDELQAYRHNHHHHHHC

SEQ ID NO: 169 - protein, 117 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = A, D, E, G, N, Q, S or V
  VARIANT 11: note = A, E, W or Y
  VARIANT 12: note = D, G, H, I, N, T, V, W or Y
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 28: note = Any amino acid
  VARIANT 29: note = Any amino acid
  VARIANT 30: note = Any amino acid
  VARIANT 31: note = Any amino acid
  VARIANT 32: note = Any amino acid
  VARIANT 33: note = Any amino acid
  VARIANT 34: note = Any amino acid
  VARIANT 35: note = Any amino acid
  VARIANT 36: note = Any amino acid
  VARIANT 37: note = Any amino acid
  VARIANT 38: note = Any amino acid
  VARIANT 39: note = Any amino acid
  VARIANT 40: note = Any amino acid
  VARIANT 41: note = Any amino acid
  VARIANT 45: note = E or W
  VARIANT 46: note = D, E or H
  VARIANT 49: note = T, V, W
  VARIANT 50: note = Any amino acid
  VARIANT 51: note = Any amino acid
  VARIANT 52: note = Any amino acid
  VARIANT 53: note = Any amino acid
  VARIANT 54: note = Any amino acid
  VARIANT 55: note = Any amino acid
  VARIANT 56: note = Any amino acid
  VARIANT 57: note = Any amino acid
  VARIANT 58: note = Any amino acid
  VARIANT 59: note = Any amino acid
  VARIANT 60: note = Any amino acid
  VARIANT 61: note = Any amino acid
  VARIANT 62: note = Any amino acid
  VARIANT 63: note = Any amino acid
  VARIANT 64: note = Any amino acid
  VARIANT 65: note = Any amino acid
  VARIANT 66: note = Any amino acid
  VARIANT 67: note = Any amino acid
  VARIANT 68: note = Any amino acid
  VARIANT 69: note = Any amino acid
  VARIANT 70: note = Any amino acid
  VARIANT 71: note = Any amino acid
  VARIANT 72: note = Any amino acid
  VARIANT 73: note = Any amino acid
  VARIANT 74: note = Any amino acid
  VARIANT 75: note = Any amino acid
  VARIANT 76: note = A, H, I, T or V
  VARIANT 79: note = A, F, N, S or R
  VARIANT 83: note = F, I, H or W
  VARIANT 84: note = Any amino acid
  VARIANT 85: note = Any amino acid
  VARIANT 86: note = Any amino acid
  VARIANT 87: note = Any amino acid
  VARIANT 88: note = Any amino acid
  VARIANT 89: note = Any amino acid
  VARIANT 90: note = Any amino acid
  VARIANT 91: note = Any amino acid
  VARIANT 92: note = Any amino acid
  VARIANT 93: note = Any amino acid
  VARIANT 94: note = Any amino acid
  VARIANT 95: note = Any amino acid
  VARIANT 96: note = Any amino acid
  VARIANT 97: note = Any amino acid
  VARIANT 98: note = Any amino acid
  VARIANT 99: note = Any amino acid
  VARIANT 100: note = Any amino acid
  VARIANT 101: note = Any amino acid
  VARIANT 102: note = Any amino acid
  VARIANT 103: note = Any amino acid
  VARIANT 104: note = Any amino acid
  VARIANT 105: note = Any amino acid
  VARIANT 106: note = Any amino acid
  VARIANT 107: note = Any amino acid
  VARIANT 108: note = Any amino acid
  VARIANT 109: note = Any amino acid
  VARIANT 110: note = A, L, T or V
  VARIANT 113: note = A, D, E, H, Q, R, S or Y
  VARIANT 114: note = A, E, I, L, N or Q
  VARIANT 117: note = A, D, E, H, N, R, S, V or Y
XXXXXXXXRWXXXXXXXXXXXXXXXXXXXXXXXXXXXXXXXWAWXXLGXXXXXXXXXXXXXXXXXXXXXXXXXXXXAWXRLGXXXXXXXXXXXXXXXXXXXXXXXXXXXXARXXLEX

SEQ ID NO: 170 - protein, 143 aa, synthetic construct
MGDEADRWYDQGAAYAARGDYDRAIEYYERALELDPNDAWAWWELGVAYAARGDYDRAIEYYQRALELDPNDAVAWARLGHAYAARGDYDRAIEYYQRALELDPNNEAARSNLEYARRAAACGLNDIFEAQKIEWHEHHHHHH

SEQ ID NO: 171 - protein, 143 aa, synthetic construct
MGDEADRWYYQGAAYAARGDYDRAIEYYERALELDPNNAWAWWELGVAYAARGDYDRAIEYYQRALELDPNDAVAWARLGHAYAARGDYDRAIEYYQRALELDPNNEAARSNLEYARRAAACGLNDIFEAQKIEWHEHHHHHH

SEQ ID NO: 172 - protein, 143 aa, synthetic construct
MGDEADRWYDQGAAYAARGDYDRAIEYYQRALELDPNNAWAWWELGVAYAARGDYDRAIEYYQRALELDPNDAVAWSRLGHAYAARGDYDRAIEHYQRALELDPNNEVARHLLEVARRAAACGLNDIFEAQKIEWHEHHHHHH

SEQ ID NO: 173 - protein, 143 aa, synthetic construct
MGDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAAACGLNDIFEAQKIEWHEHHHHHH

SEQ ID NO: 174 - protein, 143 aa, synthetic construct
MGDEADRWYDQGAAYAARGDYDRAIEYYERALELDPNNAWAWWELGVAYAARGDYDRAIEYYQRALELDPNDAVAWARLGHAYAARGDYDRAIEYYQRALELDPNNEAARSNLEYARRAAACGLNDIFEAQKIEWHEHHHHHH

SEQ ID NO: 175 - protein, 143 aa, synthetic construct
MGDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVASRAAACGLNDIFEAQKIEWHEHHHHHH

SEQ ID NO: 176 - protein, 143 aa, synthetic construct
MGDEGDRWYYQGAAYAARGDYDRAIEYYERALELDPNNAWAWWELGVAYAARGDYDRAIEYYQRALELDPNNTVAWARLGHAYAARGDYDRAIEYYQRALELDPNNEAARSNLEYSPPAAACGLNDIFEAQKIEWHEHHHHHH

SEQ ID NO: 177 - protein, 143 aa, synthetic construct
MGDEADRWYYQGAAYAARGDYDRALEYYERALELDPSNAWAWWELGVAYAARGDYDRAIEYYQRALELDPNDAVAWARLGHAYAARGDYDRAIEYYQRALELDPNNEAARSNLEYARRAAACGLNDIFEAQKIEWHEHHHHHH

SEQ ID NO: 178 - protein, 143 aa, synthetic construct
MGDEADRWYYQGAAYAARGDYDRAIEYYERALELDPNNAWAWWELGVAYAARGDYDRAIEYYQRALELDPNNTVAWARLGHAYAARGDYDRAIEYYQRALELDPNNEAARSNLEYARRAAACGLNDIFEAQKIEWHEHHHHHH

SEQ ID NO: 179 - protein, 143 aa, synthetic construct
MGDEADRWYYRGAAYAARGDYDRAIEYYERALELDPNNAWAWWELGVAYAARGDYDRAIEYYQRALELDPNNTVAWARLGHAYAARGDYDRAIEYYQRALELDPNNEAARSNLEYARRAAACGLNDIFEAQKIEWHEHHHHHH

SEQ ID NO: 180 - protein, 143 aa, synthetic construct
MGDEADRWYDQGAAYAARGDYDRAIEYYERALELDPNNAWAWWELGAAYAARGDYDRAIEYYQRALELDPNNAVAWSRLGHAYAARGDYDRAIEYYQRALELDPNNEVARHLLEVARRAAACGLNDIFEAQKIEWHEHHHHHH

SEQ ID NO: 181 - protein, 143 aa, synthetic construct
MGDEAERWYDLGAAFAARGDYDRAIEYYQRALELDPNDAWAWWELGVAYAARGDYDRAIEYYQRALELDPNNAVAWFRLGWAYAERGDYDRAIEYYQRALELDPNNELARSELENARRAAACGLNDIFEAQKIEWHEHHHHHH

SEQ ID NO: 182 - protein, 143 aa, synthetic construct
MGDEAERWYDLGAAYAARGDYDRAIEYYQRALELDPNDAWAWWELGVAYAARGDYDRAIEYYQRALELDPNNAVAWFRLGWAYAARGDYDRAIEYYQRALELDPNNELARSELENARRAAACGLNDIFEAQKIEWHEHHHHHH

SEQ ID NO: 183 - protein, 143 aa, synthetic construct
MGDEAERWYDLGAAYAARGDYDRAIEYYQRALELDPNDAWAWWELGVAYAARGDYDRAIEYYQRALELDPNNAVAWFRLGWAYAERGDYDRAIEYYQRALELDPNNELARSELENARRAAACGLNDIFEAQKIEWHEHHHHHH

SEQ ID NO: 184 - protein, 143 aa, synthetic construct
MGDEAERWYDLGAAYAARGDYDRAIEYYQRALELDPNNAWAWWELGVAYAARGDYDRAIEYYQRALELDPNDAVAWFRLGWAYAARGDYDRAIEYYQRALELDPNNELARSELVNARRAAACGLNDIFEAQKIEWHEHHHHHH

SEQ ID NO: 185 - protein, 143 aa, synthetic construct
  VARIANT 13: note = Any amino acid
MGDEAERWYDLGXAYAARGDYDRAIEYYQRALELDPNDAWAWWELGVAYAARGDYDRAIEYYQRALELDPNNAVAWFRLGWAYAERGDYDRAIEYYQRALELDPNNELARSELENARRAAACGLNDIFEAQKIEWHEHHHHHH

SEQ ID NO: 186 - protein, 143 aa, synthetic construct
MGDEAERWYDLGAAYAARGDFDRAIEYYQRALELDPNDAWAWWELGVAYAARGDYDRAIEYYQRALELDPNNAVAWFRLGWAYAERGDYDRAIEYYQRALELDPNNELARSELENARRAAACGLNDIFEAQKIEWHEHHHHHH

SEQ ID NO: 187 - protein, 143 aa, synthetic construct
MGDEAERWYDLGAAYAARGDYDRAIEYYQRALELDPNDAWAWWELGVAYAARGDYDRAIEYYQRTLELDPNNAVAWFRLGWAYAERGDYDRAIEYYQRALELDPNNELARSELENARRAAACGLNDIFEAQKIEWHEHHHHHH

SEQ ID NO: 188 - protein, 143 aa, synthetic construct
MGDEAERWYDLGAAYAARGDYDRAIEYYQRALELDPNNAWAWWELGVAYAARGDYDRAIEYYQRALELDPNDAVAWFRLGWAYAARGDYDRAIEYYQRALELDPNNELARSELENARRAAACGLNDIFEAQKIEWHEHHHHHH

SEQ ID NO: 189 - protein, 143 aa, synthetic construct
MGDEAERWYDLGAAYAARGDYDRAIEYYQRALELDPNDAWAWWELGVAYAARGDYDRAIEYYQRALELDPNNAVAWFRLGWAYAARGDYDRAIEYYQRALELDPNNELARSELVNARRAAACGLNDIFEAQKIEWHEHHHHHH

SEQ ID NO: 190 - protein, 143 aa, synthetic construct
MGDEASRWYHLGVAYAARGDYDRAIEYYQRALELDPNNAWAWWELGVAYAARGDYDRAIEYYQRALELDPNNAIAWSRLGHAYAARGDYDRAIEYYQRALELDPNNEVARHLLEYARRAAACGLNDIFEAQKIEWHEHHHHHH

SEQ ID NO: 191 - protein, 246 aa, synthetic construct
MGDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRLEDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVYRRAAAHHHHHHC

SEQ ID NO: 192 - protein, 128 aa, synthetic construct
MGDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAAAHHHHHHC

SEQ ID NO: 193 - protein, 251 aa, synthetic construct
MGDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRLEGGGGSDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAAAHHHHHHC

SEQ ID NO: 194 - protein, 186 aa, synthetic construct
MAQHDKIQQAADKEILHLPNLTEEQRNKFRQSLRDDPSVSAEILAEAKKLNDAQAPKAMGDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAAAHHHHHHC

SEQ ID NO: 195 - protein, 242 aa, synthetic construct
MAQHDKIQQAADKEILHLPNLTEEQRNKFRQSLRDDPSVSAEILAEAKKLNDAQAPKAQHDKIQQAADKEILHLPNLTEEQRNKFRQSLRDDPSVSAEILAEAKKLNDAQAPKAMGDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAAAHHHHHHC

SEQ ID NO: 196 - protein, 184 aa, synthetic construct
MGDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAAAAQHDKIQQAADKEILHLPNLTEEQRNKFRQSLRDDPSVSAEILAEAKKLNDAQAPKHHHHHHC

SEQ ID NO: 197 - protein, 240 aa, synthetic construct
MGDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAAAAQHDKIQQAADKEILHLPNLTEEQRNKFRQSLRDDPSVSAEILAEAKKLNDAQAPKAQHDKIQQAADKEILHLPNLTEEQRNKFRQSLRDDPSVSAEILAEAKKLNDAQAPKHHHHHHC

SEQ ID NO: 198 - protein, 262 aa, synthetic construct
MGDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAAADEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAAAGLNDIFEAQKIEWHEHHHHHHC

SEQ ID NO: 199 - protein, 500 aa, synthetic construct
MGDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAAADEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAMGDEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAAADEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVARRAAAGLNDIFEAQKIEWHEHHHHHHC

SEQ ID NO: 200 - protein, 178 aa, synthetic construct
MADEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVAQRAAAAQHDKIQQAADKEILHLPNLTEEQRNKFRQSLRDDPSVSAEILAEAKKLNDAQAPKC

SEQ ID NO: 201 - protein, 295 aa, synthetic construct
MADEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVAQAPKVDAKFDKEQQAARAEILHLPNLTEEQRNKFIQSLKDDPSQSANLLAEAKKLNDAQAPKVDAKFDKEQQAARAEILHLPNLTEEQRNKFIQSLKDDPSQSANLLAEAKKLNDAQAPKVDAKFDKEQQAARAEILHLPNLTEEQRNKFIQSLKDDPSQSANLLAEAKKLNDAQAPKC

SEQ ID NO: 202 - protein, 291 aa, synthetic construct
MAQHDKIQQAADKEILHLPNLTEEQRNKFRQSLRDDPSVSAEILAEAKKLNDAQAPKADEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVAQAPKAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 203 - protein, 234 aa, synthetic construct
MADEADRWYDQGAAYAARGDYDRAIEYYQRALELDPNNAWAWWELGVAYAARGDYDRAIEYYQRALELDPNDAVAWSRLGHAYAARGDYDRAIEYYQRALELDPNNEVARHLLEVAQRAAAAQHDKIQQAADKEILHLPNLTEEQRNKFRQSLRDDPSVSAEILAEAKKLNDAQAPKAQHDKIQQAADKEILHLPNLTEEQRNKFRQSLRDDPSVSAEILAEAKKLNDAQAPKC

SEQ ID NO: 204 - protein, 128 aa, synthetic construct
MGDEADRWYDQGAAYAARGDYDRAIEYYQRALELDPNNAWAWWELGVAYAARGDYDRAIEYYQRALELDPNDAVAWSRLGHAYAARGDYDRAIEHYQRALELDPNNEVARHLLEVARRAAAHHHHHHC

SEQ ID NO: 205 - protein, 246 aa, synthetic construct
MGDEADRWYDQGAAYAARGDYDRAIEYYQRALELDPNNAWAWWELGVAYAARGDYDRAIEYYQRALELDPNDAVAWSRLGHAYAARGDYDRAIEHYQRALELDPNNEVARHLLEVARRLEDEADRWYDQGAAYAARGDYDRAIEYYQRALELDPNNAWAWWELGVAYAARGDYDRAIEYYQRALELDPNDAVAWSRLGHAYAARGDYDRAIEHYQRALELDPNNEVARHLLEVSRRAAAHHHHHHC

SEQ ID NO: 206 - protein, 128 aa, synthetic construct
MGDEADRWYYQGAAYAARGDYDRAIEYYERALELDPNNAWAWWELGVAYAARGDYDRAIEYYQRALELDPNDAVAWARLGHAYAARGDYDRAIEYYQRALELDPNNEAARSNLEYARRAAAHHHHHHC

SEQ ID NO: 207 - protein, 246 aa, synthetic construct
MGDEADRWYYQGAAYAARGDYDRAIEYYERALELDPNNAWAWWELGVAYAARGDYDRAIEYYQRALELDPNDAVAWARLGHAYAARGDYDRAIEYYQRALELDPNNEAARSNLEYARRLEDEADRWYYQGAAYAARGDYDRAIEYYERALELDPNNAWAWWELGVAYAARGDYDRAIEYYQRALELDPNDAVAWARLGHAYAARGDYDRAIEYYQRALELDPNNEAARSNLEYARRAAAHHHHHRC

SEQ ID NO: 208 - protein, 183 aa, synthetic construct
MAQHDKIQQAADKEILHLPNLTEEQRNKFRQSLRDDPSVSAEILAEAKKLNDAQAPKDEADRWYYQGAAYAARGDYDRAIEYYERALELDPNNAWAWWELGVAYAARGDYDRAIEYYQRALELDPNDAVAWARLGHAYAARGDYDRAIEYYQRALELDPNNEAARSNLEYARRAAAHHHHHHC

SEQ ID NO: 209 - protein, 238 aa, synthetic construct
MAQHDKIQQAADKEILHLPNLTEEQRNKFRQSLRDDPSVSAEILAEAKKLNDAQAPKDEADRWYYQGAAYAARGDYDRAIEYYERALELDPNNAWAWWELGVAYAARGDYDRAIEYYQRALELDPNDAVAWARLGHAYAARGDYDRAIEYYQRALELDPNNEAARSNLEYARRAAAQHDKIQQAADKEILHLPNLTEEQRNKFRQSLRDDPSVSAEILAEAKKLNDAQAPKHHHHHHC

SEQ ID NO: 210 - protein, 128 aa, synthetic construct
MGDEADRWYDQGAAYAARGDYDRAIEYYERALELDPNDAWAWWELGVAYAARGDYDRAIEYYQRALELDPNDAVAWARLGHAYAARGDYDRAIEYYQRALELDPNNEAARSNLEYARRAAAHHHHHHC

SEQ ID NO: 211 - protein, 183 aa, synthetic construct
MAQHDKIQQAADKEILHLPNLTEEQRNKFRQSLRDDPSVSAEILAEAKKLNDAQAPKDEADRWYDQGAAYAARGDYDRAIEYYERALELDPNDAWAWWELGVAYAARGDYDRAIEYYQRALELDPNDAVAWARLGHAYAARGDYDRAIEYYQRALELDPNNEAARSNLEYARRAAAHHHHHHC

SEQ ID NO: 212 - protein, 143 aa, synthetic construct
MGDEADRWYDQGAAYAARGDYDRAIEYYERALELDPNDAWAWWELGVAYAARGDYDRAIEYYQRALELDPNDAVAWARLGHAYAARGDYDRAIEYYQRALELDPNNEAARANLEYARRAAAGLNDIFEAQKIEWHEHHHHHHC

SEQ ID NO: 213 - protein, 143 aa, synthetic construct
MGDEADRWYDQGAAYAARGDYDRAIEYYERALELDPNDAWAWWELGVAYAARGDYDRAIEYYQRALELDPNDAVAWARLGHAYAARGDYDRAIEYYQRALELDPNNEAARSALEYARRAAAGLNDIFEAQKIEWHEHHHHHHC

SEQ ID NO: 214 - protein, 143 aa, synthetic construct
MGDEADRWYDQGAAYAARGDYDRAIEYYERALELDPNDAWAWWELGVAYAARGDYDRAIEYYQRALELDPNDAVAWARLGHAYAARGDYDRAIEYYQRALELDPNNEAARSNLEAARRAAAGLNDIFEAQKIEWHEHHHHHHC

SEQ ID NO: 215 - protein, 143 aa, synthetic construct
MGDEADRWYDQGAAYAARGDYDRAIEYYERALELDPNDAWAWWELGVAYAARGDYDRAIEYYQRALELDPNDAVAWARLGHAYAARGDYDRAIEYYQRALELDPNNEAARAALEYARRAAAGLNDIFEAQKIEWHEHHHHHHC

SEQ ID NO: 216 - protein, 143 aa, synthetic construct
MGDEADRWYDQGAAYAARGDYDRAIEYYERALELDPNDAWAWWELGVAYAARGDYDRAIEYYQRALELDPNDAVAWARLGHAYAARGDYDRAIEYYQRALELDPNNEAARANLEAARRAAAGLNDIFEAQKIEWHEHHHHHHC

SEQ ID NO: 217 - protein, 143 aa, synthetic construct
MGDEADRWYDQGAAYAARGDYDRAIEYYERALELDPNDAWAWWELGVAYAARGDYDRAIEYYQRALELDPNDAVAWARLGHAYAARGDYDRAIEYYQRALELDPNNEAARSALEAARRAAAGLNDIFEAQKIEWHEHHHHHHC

SEQ ID NO: 218 - protein, 143 aa, synthetic construct
MGDEADRWYDQGAAYAARGDYDRAIEYYERALELDPNDAWAWWELGVAYAARGDYDRAIEYYQRALELDPNDAVAWARLGHAYAARGDYDRAIEYYQRALELDPNNEAARAALEAARRAAAGLNDIFEAQKIEWHEHHHHHHC

SEQ ID NO: 219 - protein, 143 aa, synthetic construct
MGDEADRWYDQGAAYAARGDYDRAIEYYERALELDPNNAWAWWELGVAYAARGDYDRAIEYYQRALELDPNNAVAWARLGHAYAARGDYDRAIEYYQRALELDPNNEAARSNLEYARRAAACGLNDIFEAQKIEWHEHHHHHH

SEQ ID NO: 220 - protein, 143 aa, synthetic construct
MGDEADRWYDQGAAYAARGDYDRAIEYYERALELDPNNAWAWWELGTAYAARGDYDRAIEYYQRALELDPNNAVAWSRLGHAYAARGDYDRAIEYYQRALELDPNNEVARHLLEVARRAAACGLNDIFEAQKIEWHEHHHHHH

SEQ ID NO: 221 - protein, 222 aa, synthetic construct
  VARIANT 1..74: note = This region may be present or absent, and when present comprises M, MDE, MGGGGSAAAGDE, MGDE, MGHHHHHHDE, MGLAEAAAREAAARAADE, or MAWAEFRQRLAAIRTRLEALGGSEAELAAFEREIA AFESELQAYAGAGNPEVEALRREAAAIRDELQAYRHNDE
  VARIANT 91: note = F or Y
  VARIANT 95: note = I or T
  VARIANT 97: note = Y or F
  VARIANT 99: note = R or Q
  VARIANT 101: note = T or A
  VARIANT 104: note = L or H
  VARIANT 117: note = V or I
  VARIANT 140: note = L, P or T
  VARIANT 155: note = T or A
  VARIANT 181: note = D or G
  VARIANT 189: note = A or V
  VARIANT 190..222: note = This region may be present or absent, and when present comprises a cysteine, RRC, RRCHHHHHH, or RRGQAGQGGGSGLNDIFEAQKIEWHECHHHHHH
XXXXXXXXXXXXXXXXXXXXXXXXXXXXXXXXXXXXXXXXXXXXXXXXXXXXXXXXXXXXXXXXXXXXXXXXXXAERWYDLGAAYAARGDXDRAXEXYXRXLEXDPNDAWAWWELGXAYAARGDYDRAIEYYQRALELDXNNAVAWARLGIAYAXRGDYDRAIEYYQRALELDPNNEVARXALEYARRXXXXXXXXXXXXXXXXXXXXXXXXXXXXXXXXXX

SEQ ID NO: 222 - protein, 10 aa, synthetic construct
MGHHHHHHDE

SEQ ID NO: 223 - protein, 18 aa, synthetic construct
MGLAEAAAREAAARAADE

SEQ ID NO: 224 - protein, 9 aa, synthetic construct
RRCHHHHHH

SEQ ID NO: 225 - protein, 5 aa, synthetic construct
GGGGG

SEQ ID NO: 226 - protein, 8 aa, synthetic construct
GGGGGGGG

SEQ ID NO: 227 - protein, 12 aa, synthetic construct
MGGGGSAAAGDE

SEQ ID NO: 228 - protein, 74 aa, synthetic construct
MAWAEFRQRLAAIRTRLEALGGSEAELAAFEREIAAFESELQAYAGAGNPEVEALRREAAAIRDELQAYRHNDE

SEQ ID NO: 229 - protein, 33 aa, synthetic construct
RRGQAGQGGGSGLNDIFEAQKIEWHECHHHHHH

SEQ ID NO: 230 - protein, 4 aa, synthetic construct
MGDE

SEQ ID NO: 231 - protein, 222 aa, synthetic construct
  VARIANT 1..74: note = This region may be present or absent, and when present comprises M, MDE, MGGGGSAAAGDE, MGDE, MGHHHHHHDE, MGLAEAAAREAAARAADE, or MAWAEFRQRLAAIRTRLEALGGSEAELAAFEREIA AFESELQAYAGAGNPEVEALRREAAAIRDELQAYRHNDE
  VARIANT 91: note = F or Y
  VARIANT 95: note = I or T
  VARIANT 97: note = Y or F
  VARIANT 99: note = R or Q
  VARIANT 101: note = T or A
  VARIANT 104: note = L or H
  VARIANT 117: note = V or I
  VARIANT 140: note = L, P or T
  VARIANT 155: note = T or A
  VARIANT 181: note = D or G
  VARIANT 190..222: note = This region may be present or absent, and when present comprises a cysteine, RRC, RRCHHHHHH, or RRGQAGQGGGSGLNDIFEAQKIEWHECHHHHHH
XXXXXXXXXXXXXXXXXXXXXXXXXXXXXXXXXXXXXXXXXXXXXXXXXXXXXXXXXXXXXXXXXXXXXXXXXXAERWYDLGAAYAARGDXDRAXEXYXRXLEXDPNDAWAWWELGXAYAARGDYDRAIEYYQRALELDXNNAVAWARLGIAYAXRGDYDRAIEYYQRALELDPNNEVARXALEYARRVXXXXXXXXXXXXXXXXXXXXXXXXXXXXXXXXX